(19) United States
(12) Patent Application Publication
(10) Pub. No.: US 2018/0200257 A1
(43) Pub. Date: Jul. 19, 2018

(54) METHODS FOR THE TREATMENT OF INFLAMMATORY DISORDERS

(71) Applicant: Galapagos NV, Mechelen (BE)

(72) Inventors: Piet Tom Bert Paul Wigerinck, Mechelen (BE); Gerben Albert Eleutherius Van 'T Klooster, Mechelen (BE); Frederic Paul Vanhoutte, Mechelen (BE)

(21) Appl. No.: 15/566,039

(22) PCT Filed: Mar. 31, 2016
(86) PCT No.: PCT/EP2016/057104

§ 371 Date: Oct. 12, 2017

(30) Foreign Application Priority Data
Apr. 13, 2015 (GB) 1506229.2
Apr. 15, 2015 (GB) 1506419.9
Apr. 27, 2015 (GB) 1507113.7
Jul. 29, 2015 (GB) 1513345.7
Aug. 7, 2015 (GB) 1513993.4
Dec. 7, 2015 (GB) 1521543.7

(51) Int. Cl. A61K 31/541; A61P 29/00; A61P 19/02

(52) U.S. Cl. CPC A61K 31/541; A61P 29/00; A61K 31/519; A61K 2300/00; A61P 19/02

(57) ABSTRACT

The present invention discloses compounds according to Formula I; or a pharmaceutically acceptable salt thereof, or a solvate or the salt of a solvate thereof, pharmaceutical compositions comprising the same, and methods of treatment using the same, for use in the treatment of inflammatory conditions, by administering the compound according to Formula I.

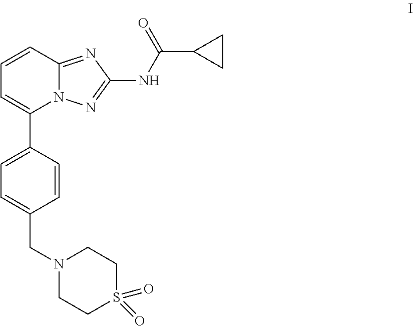 [SMILES: O=C(Nc1nc2cccc(-c3ccc(CN4CCS(=O)(=O)CC4)cc3)n2n1)C1CC1]

### FIELD OF THE INVENTION

The present invention relates to the medical use of the compound of the invention according to Formula I for the treatment of inflammatory conditions. In particular, the compound inhibits JAK, a family of tyrosine kinases, and more particularly JAK1. The present invention also provides methods for the prophylaxis and/or treatment of diseases including inflammatory conditions by administering the compound of the invention according to Formula I.

Janus kinases (JAKs) are cytoplasmic tyrosine kinases that transduce cytokine signaling from membrane receptors to STAT transcription factors. Four JAK family members are described, JAK1, JAK2, JAK3 and TYK2. Upon binding of the cytokine to its receptor, JAK family members auto- and/or transphosphorylate each other, followed by phosphorylation of STATs that then migrate to the nucleus to modulate transcription. JAK-STAT intracellular signal transduction serves the interferons, most interleukins, as well as a variety of cytokines and endocrine factors such as EPO, TPO, GH, OSM, LIF, CNTF, GM-CSF and PRL (Vainchenker et al., 2008).

JAK1 is a target in the immuno-inflammatory disease area. JAK1 heterodimerizes with the other JAKs to transduce cytokine-driven pro-inflammatory signaling. Therefore, inhibition of JAK1 is of interest for immuno-inflammatory diseases with pathology-associated cytokines that use JAK1 signaling, such as IL-6, IL-4, IL-5, IL-12, IL-13, IL-23, or IFNgamma, as well as for other diseases driven by JAK-mediated signal transduction.

### BACKGROUND OF THE INVENTION

The degeneration of cartilage is the hallmark of various diseases, among which rheumatoid arthritis and osteoarthritis are the most prominent. Rheumatoid arthritis (RA) is a chronic joint degenerative disease, characterized by inflammation and destruction of the joint structures. When the disease is unchecked, it leads to substantial disability and pain due to loss of joint functionality and even premature death. The aim of a RA therapy, therefore, is not only to slow down the disease but to attain remission in order to stop the joint destruction. Besides the severity of the disease outcome, the high prevalence of RA (˜0.8% of adults are affected worldwide) means a high socio-economic impact. (For reviews on RA, we refer to Smolen and Steiner, 2003, Lee and Weinblatt, 2001, Choy and Panayi, 2001, O'Dell, 2004 and Firestein, 2003.

JAK1 is implicated in intracellular signal transduction for many cytokines and hormones. Pathologies associated with any of these cytokines and hormones can be ameliorated by JAK1 inhibitors. Hence, several allergy, inflammation and autoimmune disorders might benefit from treatment with compounds described in this invention including rheumatoid arthritis, systemic lupus erythematosus, juvenile idiopathic arthritis, osteoarthritis, asthma, chronic obstructive pulmonary disease (COPD), tissue fibrosis, eosinophilic inflammation, eosophagitis, inflammatory bowel diseases (e.g. Crohn's disease, ulcerative colitis), transplant, graft-versus-host disease, psoriasis, myositis, psoriatic arthritis, ankylosing spondylitis, juvenile idiopathic arthritis, and multiple sclerosis (Kopf et al., 2010).

Psoriasis is a disease that can affect the skin. The cause of psoriasis is not fully understood, however, it is believed that it is an immune mediated related disease linked to the release of cytokines, in particular TNFα, which causes inflammation and rapid reproduction of the skin cells. This hypothesis has been corroborated by the observation that immunosuppressant medication can clear psoriasis plaques (Zenz et al., 2005)

Psoriasis can also cause inflammation of the joints, which is known as psoriatic arthritis. Between 10-30% of all people with psoriasis also have psoriatic arthritis (Committee for Medicinal Products for Human Use (CHMP) (18 Nov. 2004). “Guideline on Clinical Investigation of Medicinal Products indicated for the treatment of Psoriasis”). Because of its chronic recurrent nature, psoriasis is a challenge to treat. It has recently been demonstrated that inhibition of JAK could result in successful improvement of the psoriatic condition. (Punwani et al., 2012).

Inflammatory bowel disease (IBD) is a group of inflammatory conditions of the colon and small intestine. Recently, it has been found via genome-wide association (GWAS) studies that T cell protein tyrosine phosphatase (TCPTP) is a JAK/STAT and growth factor receptor phosphatase that has been linked to the pathogenesis of type 1 diabetes, rheumatoid arthritis, and Crohn's disease by GWAS (Zikherman and Weiss, 2011). Therefore, inhibition of the JAK pathway might provide a way of treating IBD.

In inflammation haemoglobin levels are lower than in healthy individuals which is indicative of the anaemia associated with inflammatory conditions. It has been reported that, whilst treatment with JAK inhibitors is able to resolve the inflammation, which would be expected to resolve the anaemia and result in restored haemoglobin levels, the effect of JAK2 inhibition in compounds such as tofacitinib and baricitinib results in anaemia, which may limit the dose that may be used in patients (Keystone et al., 2015; Riese et al., 2010). Therefore, there is a need for selective JAK1 inhibitors which can resolve the inflammation without causing anaemia.

This issue is also exacerbated in IBD patients where the anaemia in IBD patents is aggravated by the associated blood loss in the stomach or intestine which cannot be matched by duodenal iron absorption, creating a negative iron balance. As a result, despite new drug developments, one third of IBD patients still have haemoglobin levels below 12 g/dl (Gasche et al., 2004).

The current therapies are not satisfactory and therefore there remains a need to identify specific methods for the treatment of inflammatory conditions which provide clinical benefit whilst minimizing potential side effects.

The compound of the invention, cyclopropanecarboxylic acid {5-[4-(1,1-dioxo-thiomorpholin-4-ylmethyl)-phenyl]-[1,2,4]triazolo[1,5-a]pyridin-2-yl}-amide (Compound 1), is disclosed in WO2010/149769 (Menet and Smits, 2010) and has the chemical structure shown below:

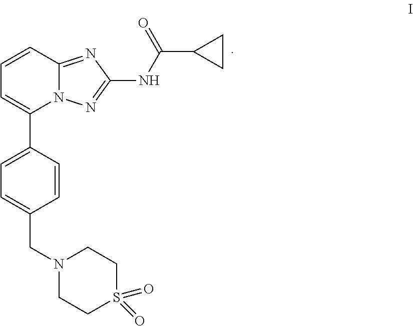 [SMILES: O=C(Nc1nc2cccc(-c3ccc(CN4CCS(=O)(=O)CC4)cc3)n2n1)C1CC1]

Compound 1 is a JAK inhibitor, more particularly a JAK1 inhibitor, and was disclosed as being useful in the treatment of inflammatory conditions, autoimmune diseases, proliferative diseases, allergy, transplant rejection, diseases involving impairment of cartilage turnover, congenital cartilage malformations, and/or diseases associated with hypersecretion of IL6 or interferons.

Compound 1 was previously dosed in clinical testing for a period of 4 weeks in patients who have not previously received biological treatments for RA, where the patients received concomitant methotrexate therapy. Methotrexate is a well-established and trusted treatment for inflammatory conditions and autoimmune diseases (e.g. RA), in particular the ubiquitous nature of methotrexate therapies for these conditions has resulted in authorities and ethical committees for clinical trials being resistant to the use of “pure” placebo arms (i.e. no therapy at all) in randomized clinical trials of new agents tested for the treatment of these conditions (Kaltsonoudis et al.). For that reason, the treatment of RA patient classified as methotrexate non-responders or insufficient responders is usually the first challenge for emerging drugs in this area. Nevertheless, in order to improve patient life condition and compliance, single agent drug treatment are highly desirable. Indeed, single treatment may be beneficial in reducing drug-drug interaction, especially in patients under additional therapies.

Furthermore, JAK inhibitors have been reported to have the potential for causing anemia in patients (O'Shea et al., 2013), This unwanted side effect is a known dose-limiting issue for rheumatoid arthritis, and additionally for IBD, would clearly limit the therapeutic window, as patients are already at an increased risk of anemia. Therefore, there is a need for JAK inhibitors for use in the treatment of inflammatory conditions which have a reduced risk or no risk of anaemia.

### SUMMARY OF THE INVENTION

The present invention provides the compound of the invention according to Formula I for use in the treatment of inflammatory conditions, in particular for use in the treatment of rheumatoid arthritis or Crohn's disease when dosed alone or in combination, orally at a dose selected from 25 mg twice per day (b.i.d.), 50 mg once a day (q.d.), 50 mg b.i.d., 100 mg q.d., 100 mg b.i.d., and 200 mg q.d.

The present invention also provides methods treatment of inflammatory conditions by administering the compound of the invention orally at a dose selected from 25 mg twice per day (b.i.d.), 50 mg once a day (q.d.), 50 mg b.i.d., 100 mg q.d., 100 mg b.i.d., and 200 mg q.d.

Accordingly, in a first aspect of the invention, the compound of the invention having according to Formula I shown below:

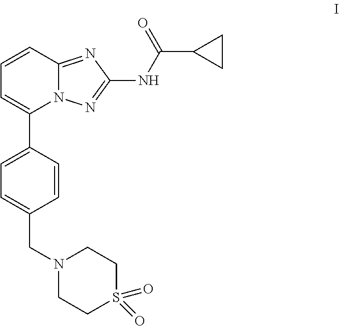 [SMILES: O=C(Nc1nc2cccc(-c3ccc(CN4CCS(=O)(=O)CC4)cc3)n2n1)C1CC1]

is provided for use in the treatment of inflammatory conditions where the compound of the invention is dosed orally at a dose of between 25 mg to 400 mg, administered once or twice a day. In particular, the compound of the invention is dosed orally at a dose of between 100 mg to 250 mg. More particularly, at a dose is selected from 25 mg twice per day (b.i.d.), 50 mg once a day (q.d.), 50 mg b.i.d., 100 mg q.d., 100 mg b.i.d., and 200 mg q.d.

In a particular aspect is provided the compound of the invention for use in the prophylaxis and/or treatment of rheumatoid arthritis or IBD (e.g. Crohn's disease or ulcerative colitis).

In a particular embodiment the patient does not take methotrexate concomitantly with the administration of the compound of the invention.

In a specific embodiment, the patient does not receive any other treatment for rheumatoid arthritis or IBD (e.g. Crohn's disease or ulcerative colitis) concomitantly with the administration of the compound of the invention.

In a specific embodiment, the compound of the invention is dosed as the sole treatment agent.

In an alternative aspect the patient takes methotrexate concomitantly with the administration of compound of the invention, in particular between 5-25 mg once per week of methotrexate, more particularly between 10-25 mg once per week of methotrexate, over a period greater than 4 weeks.

In an alternative aspect the patient takes methotrexate concomitantly with the administration of compound of the invention, in particular between 5-25 mg once per week of methotrexate, more particularly between 10-25 mg once per week of methotrexate, over a period greater than 8, 12, 16, or 20 weeks.

In an alternative aspect the patient takes methotrexate concomitantly with the administration of compound of the invention, in particular between 5-25 mg once per week of methotrexate, more particularly between 10-25 mg once per week of methotrexate, over a period of at least 24 weeks.

In a particular aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d. for at least 4 weeks. In particular the compound of the invention is dosed for at least 8 weeks. In particular the compound of the invention is dosed for at least 10 weeks. In particular the compound of the invention is dosed for at least 12 weeks. In particular the compound of the invention is dosed for at least 16 weeks. In particular the compound of the invention is dosed for at least 20 weeks. In particular the compound of the invention is dosed for at least 24 weeks.

In a further aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d., where an ACR20 response is seen in at least 50% of patients.

In a further aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d., where an ACR50 response is seen in at least 30% of patients.

In a further aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d., where an ACR70 response is seen in at least 10% of patients.

In a further aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d., where an ACR70 response is seen in at least 20% of patients. In yet a further aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d., where an ACR70 response is seen in at least 20% of patients after 20 weeks.

In a further aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d., where a reduction in the DAS28(CRP) score of at least 1.8 is seen after 12 weeks of treatment.

In a further aspect is provided the compound of the invention for use in the treatment of chronic inflammatory conditions, wherein the compound of the invention is dosed orally at a dose selected from 25 mg twice per day (b.i.d.), 50 mg once a day (q.d.), 50 mg b.i.d., 100 mg q.d., 100 mg b.i.d., and 200 mg q.d., where a reduction in the DAS28(CRP) score of at least 2.0 is seen after 24 weeks of treatment.

In another aspect is provided the compound of the invention for use in the treatment of Crohn's disease, wherein the compound is dosed at 200 mg q.d., and wherein a reduction in CDAI score of at least 70, at least 80, at least 90, at least 100, at least 110, at least 120, at least 130, at least 140, at least 150, at least 200 or at least 250 is seen after 10 weeks treatment. In a more particular aspect, the compound is dosed at 200 mg q.d., and the patient achieves clinical remission (CDAI score <150) after 2 weeks, after 4 weeks, after 6 weeks, or after 10 weeks . In another particular aspect, the compound is dosed at 200 mg q.d., and the patient achieves clinical remission after 10 weeks (CDAI score <150).

In another particular aspect is provided the compound of the invention for use in the treatment of Crohn's disease, wherein the compound is dosed at 200 mg q.d., and wherein prior to treatment, the Crohn's disease patient shows a CDAI score of at least 220 points, at least 250 points, at least 300 points, at least 350 points, or at least 400 points,. In a particular embodiment, prior to treatment, the Crohn's disease patient shows a CDAI score of at least 250 points, at least 300 points or at least 350 points. In a more particular embodiment, prior to treatment, the Crohn's disease patient shows a CDAI score of at least 250 points, at least 260 points, at least 270 points, at least 280 points, at least 290 points, or at least 300 points.

In one aspect, the present invention provides a compound of the invention for use in the prophylaxis and/or treatment of IBD, wherein the patient is a TNF naïve patient. In a particular aspect, the present invention provides a compound of the invention for use in the prophylaxis and/or treatment of Crohn's disease, wherein the patient is a TNF naïve patient.

In another particular aspect, the present invention provides a compound of the invention for use in the prophylaxis and/or treatment of IBD, wherein the patient is a TNF experienced patient. In another more particular aspect, the present invention provides a compound of the invention for use in the prophylaxis and/or treatment of Crohn's disease, wherein the patient is a TNF experienced patient. In a further more particular aspect, the patient is a TNF experienced patient, wherein the anti-TNF therapy is selected from infliximab, adalimumab, golimumab, certolizumab and certolizumab pegol.

In another aspect, the patient has a serum CRP level prior to starting treatment with the compound of the invention of 10 mg/L or higher.

In another aspect, prior to and/or concomitantly with treatment with the compound of the invention, the patient is administered corticosteroids. In particular, the corticosteroid is selected from hydrocortisone, methylprednisolone, prednisone, prednisolone, or budesonide. In a more particular embodiment, the patient is concomitantly receiving and/or has received a daily corticosteroid dose of 20 to 30 mg/day prednisolone/equivalent. In a most particular embodiment, the patient is receiving a daily corticosteroid dose of 20, 21, 22, 23, 24, or 25 mg/day prednisolone/equivalent.

In another aspect, prior and/or concomitantly to treatment with the compound of the invention, the patient is receiving treatment using 5-aminosalicylates. In particular, the 5-aminosalicylate is selected from sulfasalazine and mesalamine.

In a further aspect of the invention, this invention provides a method of treating a patient afflicted with a condition selected from among those listed herein which method comprises administering the compound of the invention orally at a dose selected from 25 mg twice per day (b.i.d.), 50 mg once a day (q.d.), 50 mg b.i.d., 100 mg q.d., 100 mg b.i.d., and 200 mg q.d.

In additional aspects, this invention discloses methods for synthesizing the compound of the invention, with representative synthetic protocols and pathways disclosed later on herein.

Other objects and advantages will become apparent to those skilled in the art from a consideration of the ensuing detailed description.

It will be appreciated that the compound of the invention may be metabolized to yield biologically active metabolites.

### BRIEF DESCRIPTION OF THE DRAWINGS

FIG. 1 shows the percentage of the patient population achieving an ACR20 response at the 1, 2, 4, 8 and 12 week treatment time points in Study 1. The y-axis is the percentage of patients meeting the ACR20 criteria. FIG. 1A shows: placebo (pale solid line), 50 mg q.d. (dotted line), 100 mg q.d. (dashed line) or 200 mg q.d. (dark solid line). FIG. 1B shows: placebo (pale solid line), 25 mg b.i.d. (dotted line), 50 mg b.i.d. (dashed line) or 100 mg b.i.d. (dark solid line). The results shown in the figures are calculated using the non-responder imputation (NRI), which assigns a value of nonresponse to missing data points, i.e. any patient who drops out is assumed to be a non-responder (NR).

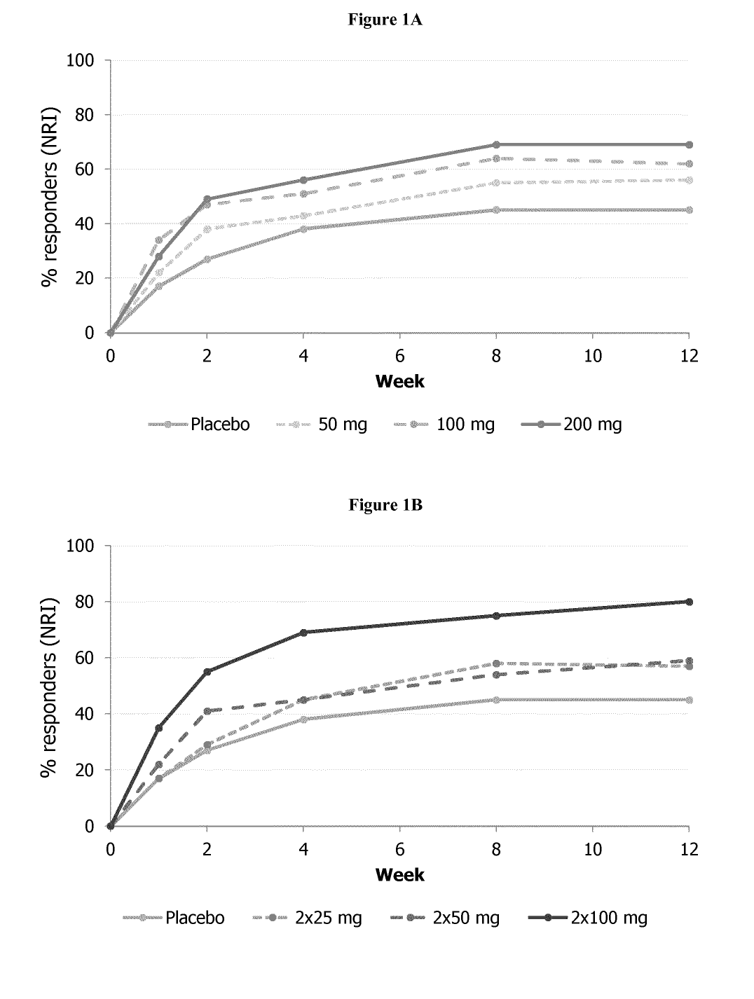

FIG. 2 shows the percentage of the patient population achieving an ACR50 response at the 1, 2, 4, 8 and 12 week treatment time points in Study 1. The y-axis is the percentage of patients meeting the ACR50 criteria. FIG. 2A shows: placebo (pale solid line), 50 mg q.d. (dotted line), 100 mg q.d. (dashed line) or 200 mg q.d. (dark solid line). FIG. 2B shows: placebo (pale solid line), 25 mg b.i.d. (dotted line), 50 mg b.i.d. (dashed line) or 100 mg b.i.d. (dark solid line). The results shown in the figures are calculated using the non-responder imputation (NRI), which assigns a value of nonresponse to missing data points, i.e. any patient who drops out is assumed to be a non-responder (NR).

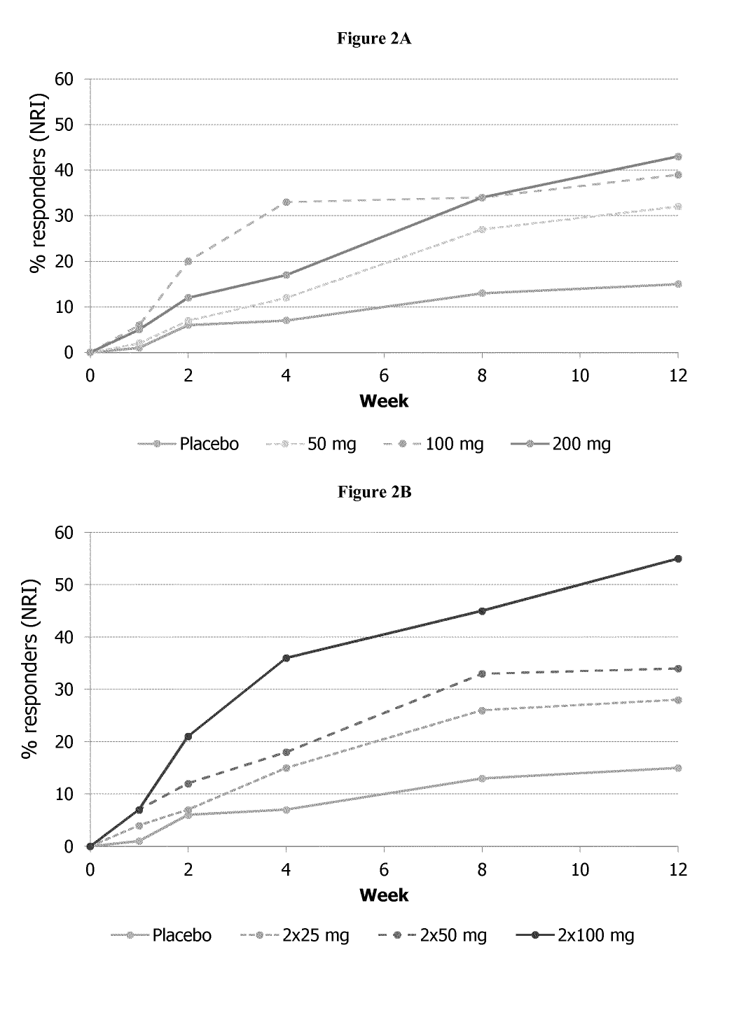

FIG. 3 shows the percentage of the patient population achieving an ACR70 response at the 1, 2, 4, 8 and 12 week treatment time points in patients in Study 1. The y-axis is the percentage of patients meeting the ACR70 criteria. FIG. 3A shows: placebo (pale solid line), 50 mg q.d. (dotted line), 100 mg q.d. (dashed line) or 200 mg q.d. (dark solid line). FIG. 3B shows: placebo (pale solid line), 25 mg b.i.d. (dotted line), 50 mg b.i.d. (dashed line) or 100 mg b.i.d. (dark solid line). The results shown in the figures are calculated using the non-responder imputation (NRI), which assigns a value of nonresponse to missing data points, i.e. any patient who drops out is assumed to be a non-responder (NR).

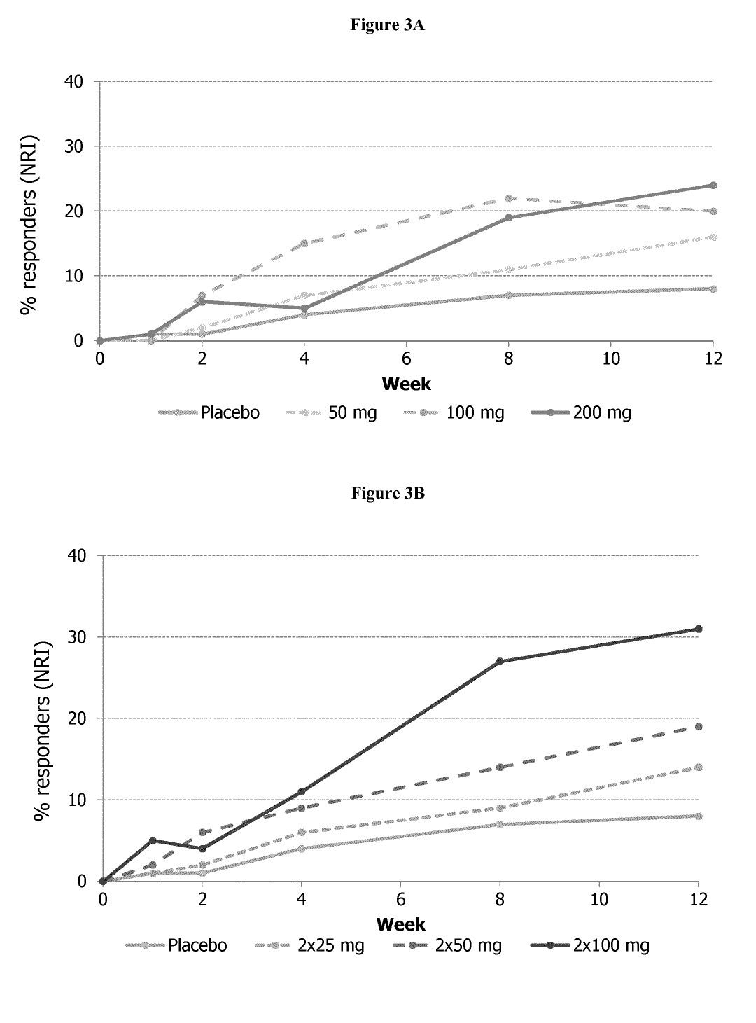

FIG. 4 shows the decrease in the DAS28(CRP) score in the patient population at the 1, 2, 4, 8 and 12 week time points for each dose in Study 1. FIG. 4A shows placebo (pale solid line), 50 mg q.d. (dotted line), 100 mg q.d. (dashed line) or 200 mg q.d. (dark solid line). FIG. 4B shows: placebo (pale solid line), 25 mg b.i.d. (dotted line), 50 mg b.i.d. (dashed line) or 100 mg b.i.d. (dark solid line). The results shown in the figures are calculated using Last-Observation-Carried-Forward (LOCF), which handles missing data by assigning the value recorded at the patient's last visit to all subsequent missed visits.

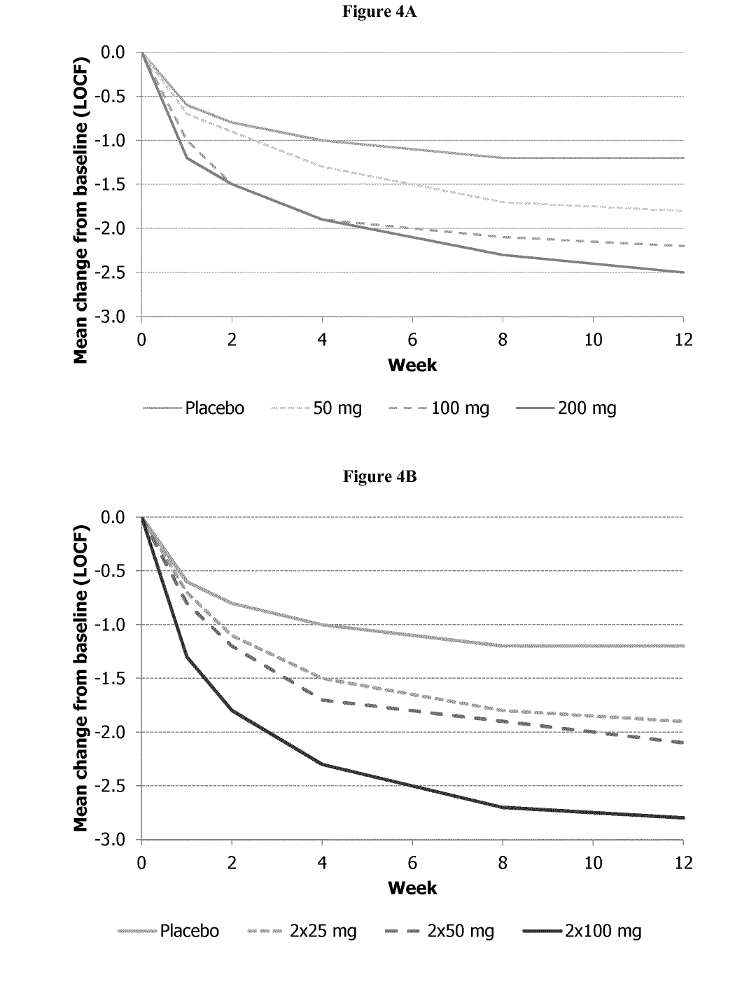

FIG. 5 shows the decrease in the serum CRP level in mg/L in the patient population at the 1, 2, 4, 8 and 12 week time points for each dose in Study 1. FIG. 5A shows placebo (pale solid line), 50 mg q.d. (dotted line), 100 mg q.d. (dashed line) or 200 mg q.d. (dark solid line). FIG. 5B shows: placebo (pale solid line), 25 mg b.i.d. (dotted line), 50 mg b.i.d. (dashed line) or 100 mg b.i.d. (dark solid line). The results shown in the figures are calculated using Last-Observation-Carried-Forward (LOCF), which handles missing data by assigning the value recorded at the patient's last visit to all subsequent missed visits.

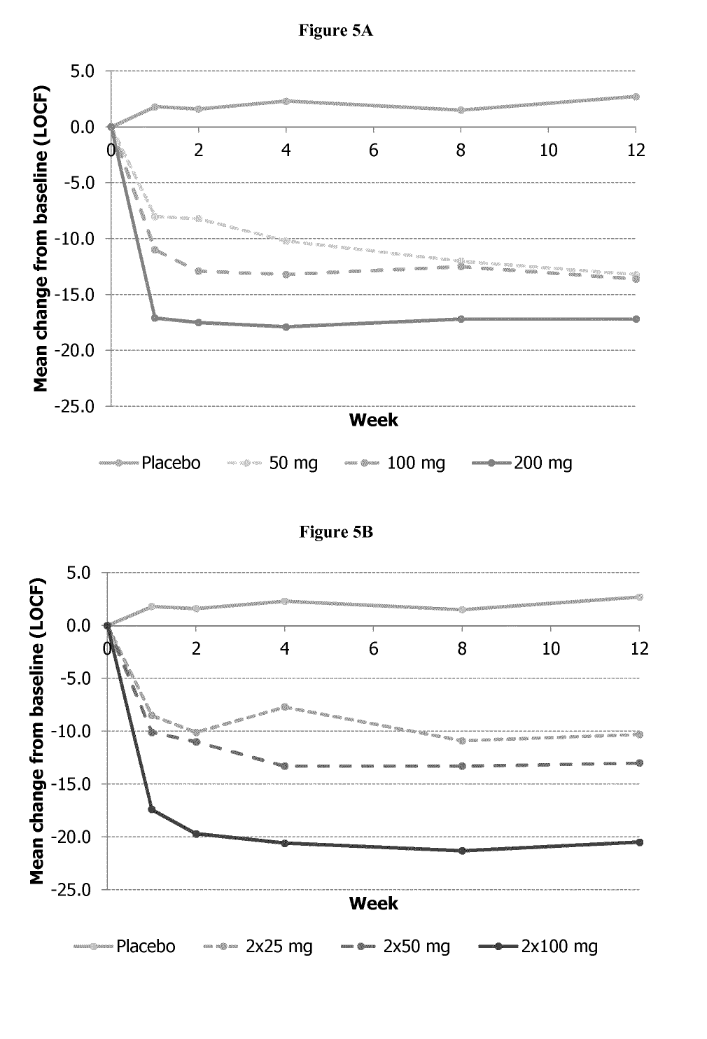

FIG. 6 shows the change in the haemoglobin levels in g/L in the patient population at the 1, 2, 4, 8 and 12 week time points for each dose in Study 1. Placebo (filled circles, solid line), 25 mg b.i.d. (open circles, broken line), 50 mg q.d. (open triangles, broken line), 50 mg b.i.d. (asterisks, solid line), 100 mg q.d. (open squares, solid line), 100 mg b.i.d. (crosses, dashed line), 200 mg q.d. (open diamonds, dashed line).

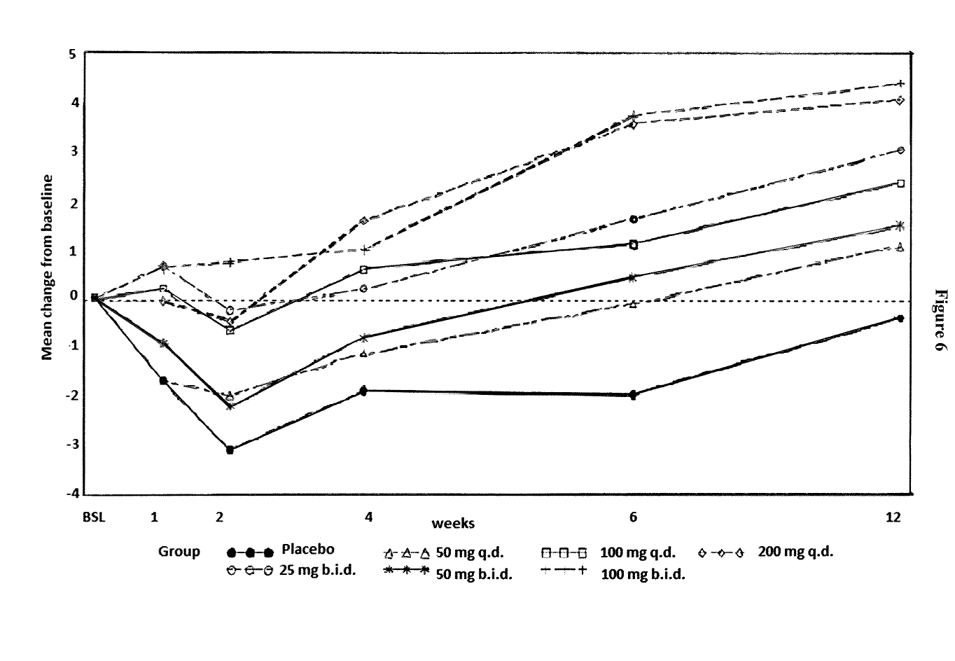

FIG. 7 shows the change in the number of neutrophils in GI/L in the patient population at the 1, 2, 4, 8 and 12 week time points for each dose in Study 1. Placebo (filled circles, solid line), 25 mg b.i.d. (open circles, broken line), 50 mg q.d. (open triangles, broken line), 50 mg b.i.d. (asterisks, solid line), 100 mg q.d. (open squares, solid line), 100 mg b.i.d. (crosses, dashed line), 200 mg q.d. (open diamonds, dashed line).

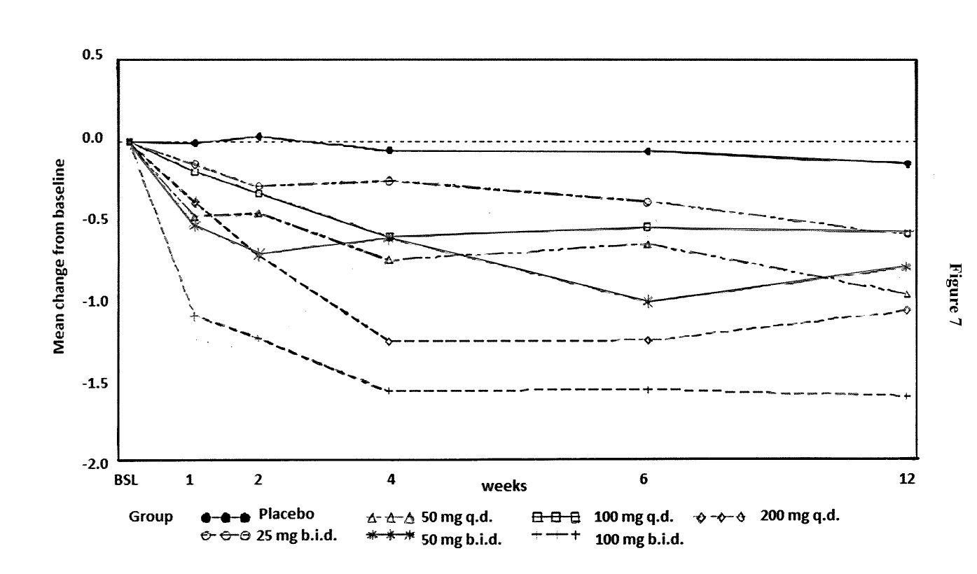

FIG. 8 shows the decrease in the Subject Global Assessment using a Visual Analog Scale (VAS), which is performed as part of the panel of ACR measurements, in the patient population at the 1, 2, 4, 8 and 12 week time points for each dose in Study 1. The scale is 100 mm, the decrease in mm is shown. FIG. 8A shows placebo (pale solid line), 50 mg q.d. (dotted line), 100 mg q.d. (dashed line) or 200 mg q.d. (dark solid line). FIG. 8B shows: placebo (pale solid line), 25 mg b.i.d. (dotted line), 50 mg b.i.d. (dashed line) or 100 mg b.i.d. (dark solid line). The results shown in the figures are calculated using Last-Observation-Carried-Forward (LOCF), which handles missing data by assigning the value recorded at the patient's last visit to all subsequent missed visits.

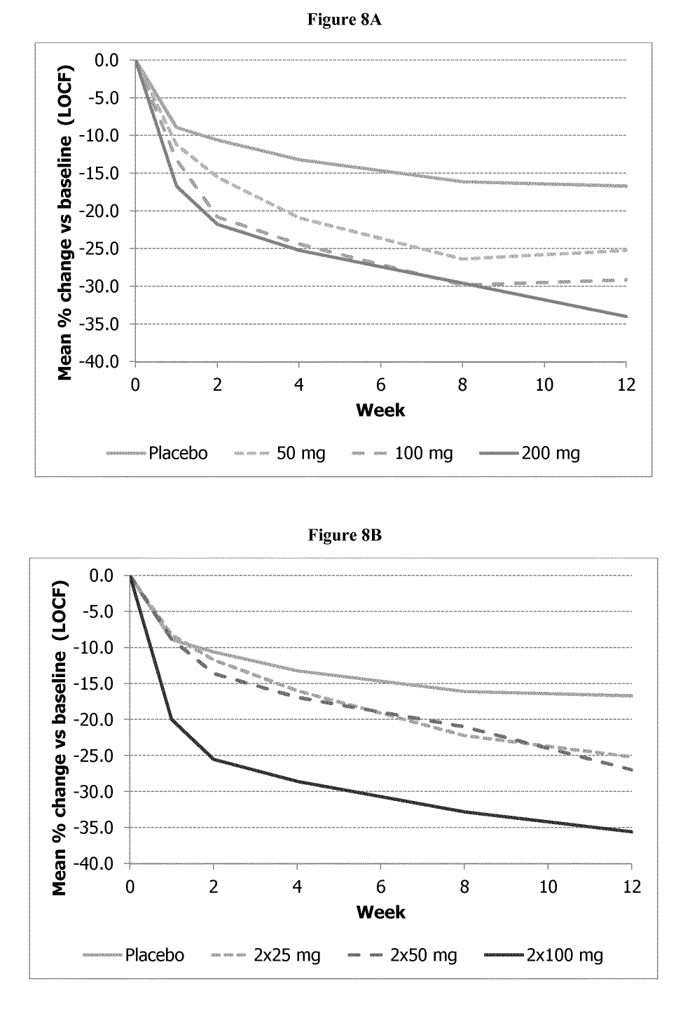

FIG. 9 shows the percentage of the patient population achieving an ACR20 response at the 1, 2, 4, 8 and 12 week treatment time points in Study 2. The y-axis is the percentage of patients meeting the ACR20 criteria. FIG. 9 shows: placebo (pale solid line), 50 mg q.d. (dashed line), 100 mg q.d. (dotted line) or 200 mg q.d. (dark solid line). The results shown in the figures are calculated using the non-responder imputation (NRI), which assigns a value of nonresponse to missing data points, i.e. any patient who drops out is assumed to be a non-responder (NR).

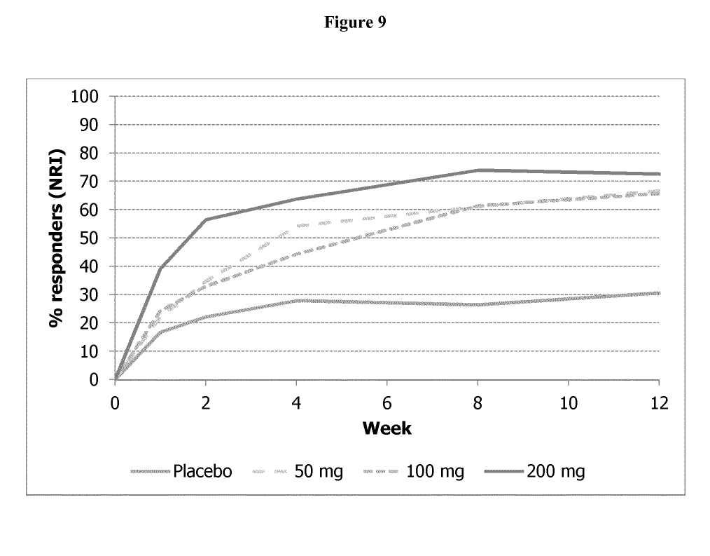

FIG. 10 shows the percentage of the patient population achieving an ACR50 response at the 1, 2, 4, 8 and 12 week treatment time points in Study 2. The y-axis is the percentage of patients meeting the ACR50 criteria. FIG. 10 shows: placebo (pale solid line), 50 mg q.d. (dashed line), 100 mg q.d. (dotted line) or 200 mg q.d. (dark solid line). The results shown in the figures are calculated using the non-responder imputation (NRI), which assigns a value of nonresponse to missing data points, i.e. any patient who drops out is assumed to be a non-responder (NR).

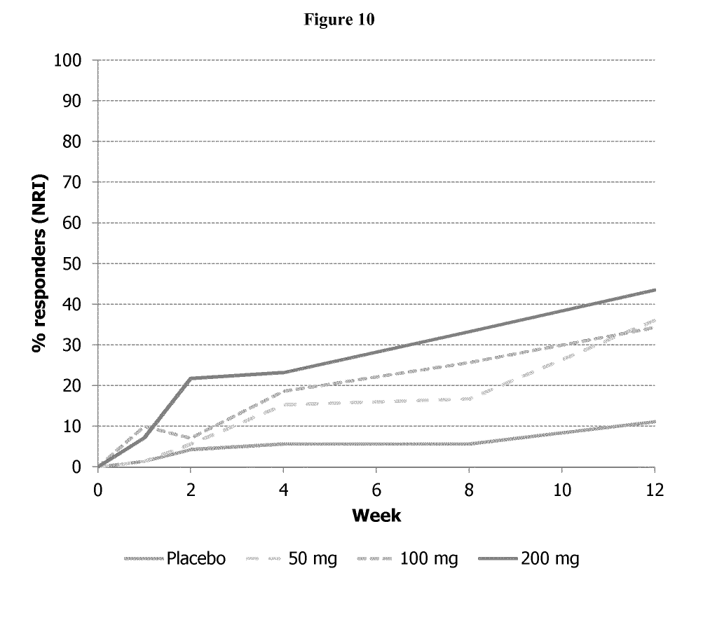

FIG. 11 shows the percentage of the patient population achieving an ACR70 response at the 1, 2, 4, 8 and 12 week treatment time points in patients in Study 2. The y-axis is the percentage of patients meeting the ACR70 criteria. FIG. 11 shows: placebo (pale solid line), 50 mg q.d. (dashed line), 100 mg q.d. (dotted line) or 200 mg q.d. (dark solid line). The results shown in the figures are calculated using the non-responder imputation (NRI), which assigns a value of nonresponse to missing data points, i.e. any patient who drops out is assumed to be a non-responder (NR).

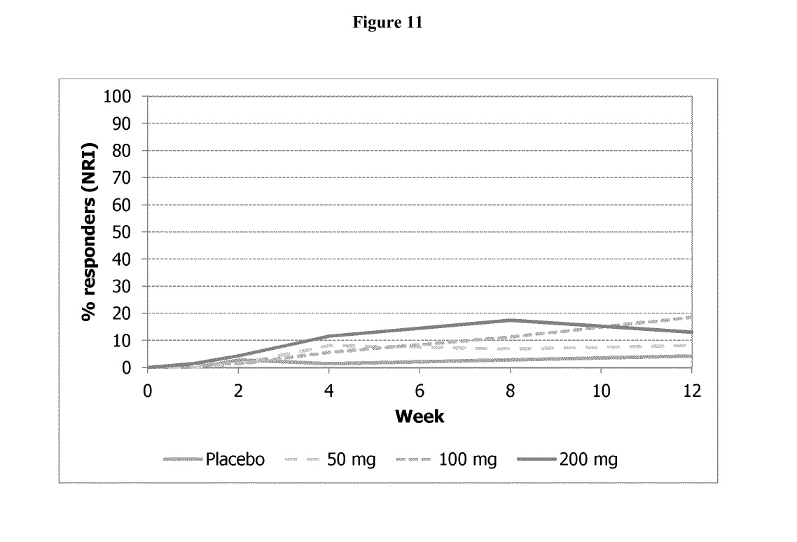

FIG. 12 shows the decrease in the DAS28(CRP) score in the patient population at the 1, 2, 4, 8 and 12 week time points for each dose in Study 2. FIG. 12 shows placebo (pale solid line), 50 mg q.d. (dotted line), 100 mg q.d. (dashed line) or 200 mg q.d. (dark solid line). The results shown in the figures are calculated using Last-Observation-Carried-Forward (LOCF), which handles missing data by assigning the value recorded at the patient's last visit to all subsequent missed visits.

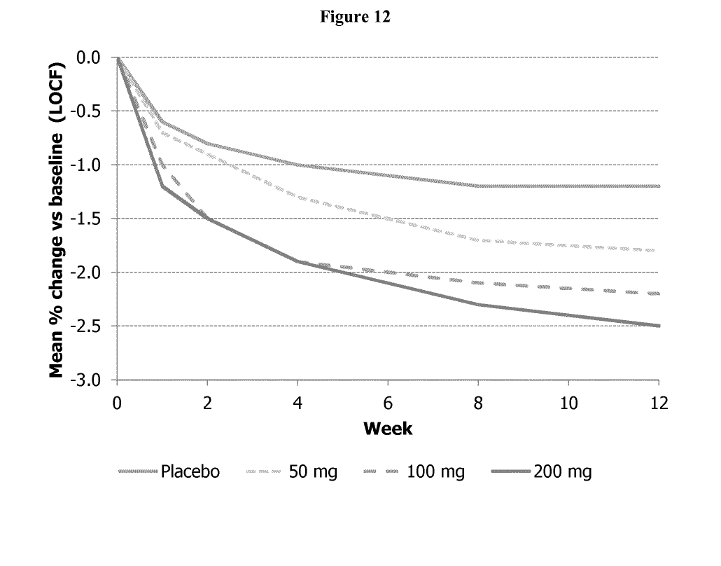

FIG. 13 shows the decrease in the serum CRP level in mg/L in the patient population at the 1, 2, 4, 8 and 12 week time points for each dose in Study 2. FIG. 13 shows placebo (pale solid line), 50 mg q.d. (dotted line), 100 mg q.d. (dashed line) or 200 mg q.d. (dark solid line). The results shown in the figures are calculated using Last-Observation-Carried-Forward (LOCF), which handles missing data by assigning the value recorded at the patient's last visit to all subsequent missed visits.

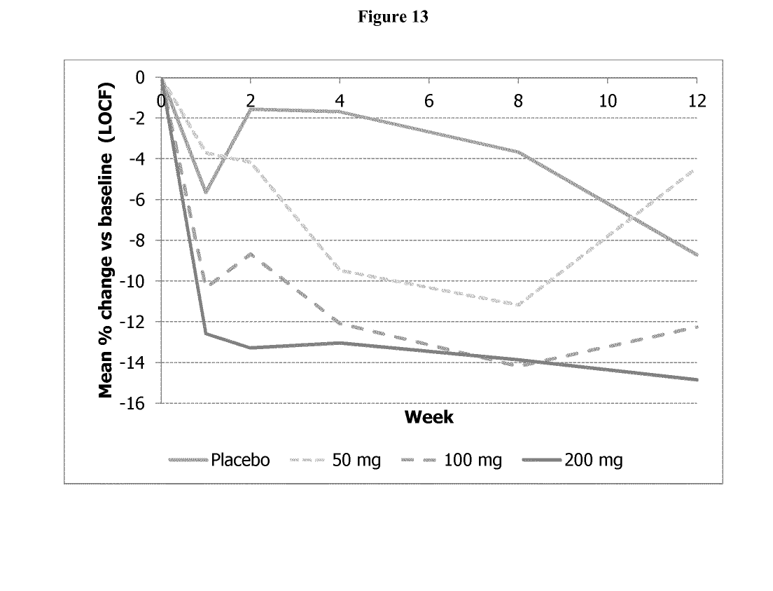

FIG. 14 shows the change in the haemoglobin levels in g/L in the patient population at the 1, 2, 4, 8 and 12 week time points for each dose in Study 2. Placebo (pale solid line), 50 mg q.d. (dotted line), 100 mg q.d. (dashed line), 200 mg q.d. (dark solid line).

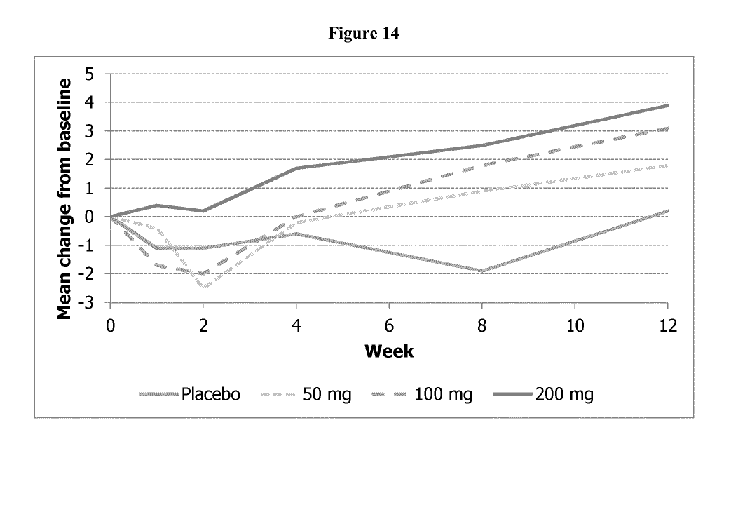

FIG. 15 shows the change in the number of neutrophils in GI/L in the patient population at the 1, 2, 4, 8 and 12 week time points for each dose in Study 2. Placebo (pale solid line), 50 mg q.d. (dotted line), 100 mg q.d. (dashed line), 200 mg q.d. (dark solid line).

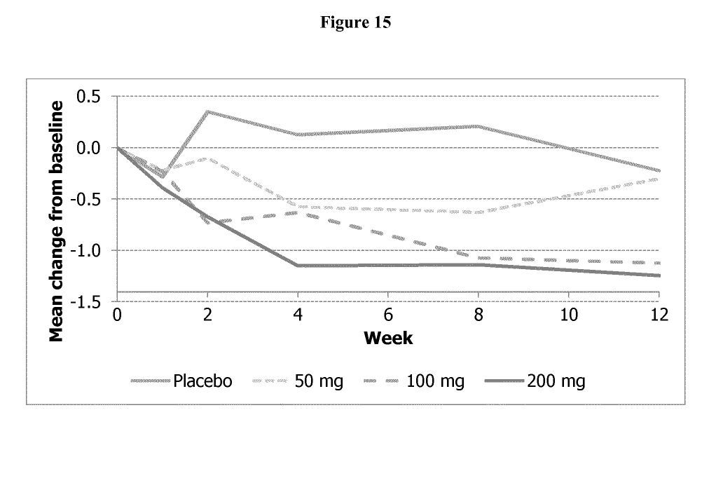

FIG. 16 shows the decrease in the Subject Global Assessment using a Visual Analog Scale (VAS), which is performed as part of the panel of ACR measurements, in the patient population at the 1, 2, 4, 8 and 12 week time points for each dose in Study 2. The scale is 100 mm, the decrease in mm is shown. FIG. 16 shows placebo (pale solid line), 50 mg q.d. (dotted line), 100 mg q.d. (dashed line) or 200 mg q.d. (dark solid line). The results shown in the figures are calculated using Last-Observation-Carried-Forward (LOCF), which handles missing data by assigning the value recorded at the patient's last visit to all subsequent missed visits.

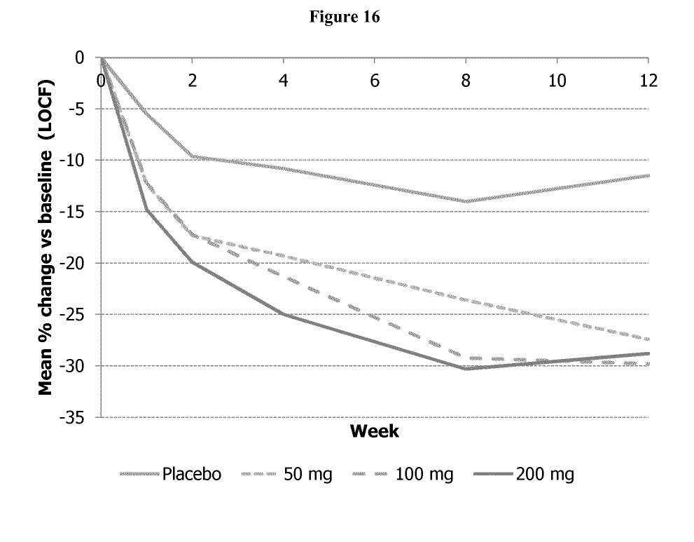

FIG. 17 shows the percentage of the patient population achieving an ACR20 response at the 1, 2, 4, 8, 12, 16, 20 and 24 week treatment time points in Study 1. The y-axis is the percentage of patients meeting the ACR20 criteria. At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. Subjects who switched treatment at week 12 were handled as if they discontinued at week 12 for the purpose of statistical analysis, whereas subjects in the other groups maintained their randomized treatment until Week 24. Consequently, the data reported from week 16 to week 24 only refers to the data for the subjects continuing on the same treatment course from week 0 to week 24. FIG. 17A shows: placebo (diamonds), 50 mg q.d. (squares), 100 mg q.d. (triangles) or 200 mg q.d. (tilted crosses). FIG. 17B shows: placebo (diamonds), 25 mg b.i.d. (asterisk), 50 mg b.i.d. (circles) or 100 mg b.i.d. (vertical crosses). The results shown in the figures are calculated using the non-responder imputation (NRI), which assigns a value of nonresponse to missing data points, i.e. any patient who drops out is assumed to be a non-responder (NR).

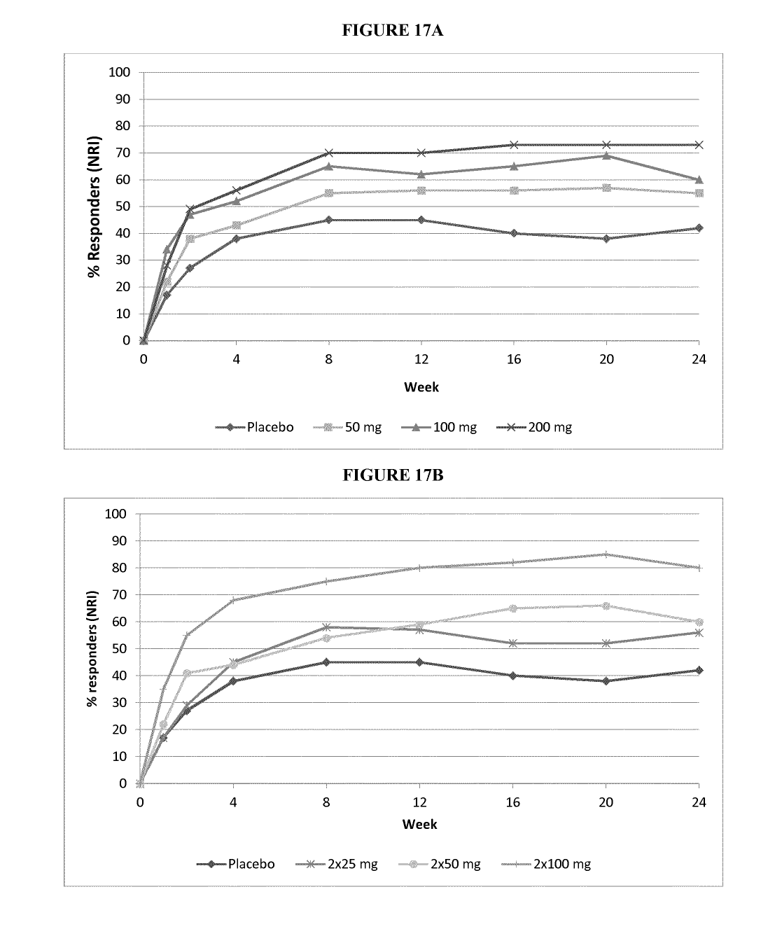

FIG. 18 shows the percentage of the patient population achieving an ACR50 response at the 1, 2, 4, 8, 12, 16, 20 and 24 week treatment time points in Study 1. The y-axis is the percentage of patients meeting the ACR50 criteria. At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. Subjects who switched treatment at week 12 were handled as if they discontinued at week 12 for the purpose of statistical analysis, whereas subjects in the other groups maintained their randomized treatment until Week 24. Consequently, the data reported from week 16 to week 24 only refers to the data for the subjects continuing on the same treatment course from week 0 to week 24. FIG. 18A shows: placebo (diamonds), 50 mg q.d. (squares), 100 mg q.d. (triangles) or 200 mg q.d. (tilted crosses). FIG. 18B shows: placebo (diamonds), 25 mg b.i.d. (asterisk), 50 mg b.i.d. (circles) or 100 mg b.i.d. (vertical crosses). The results shown in the figures are calculated using the non-responder imputation (NRI), which assigns a value of nonresponse to missing data points, i.e. any patient who drops out is assumed to be a non-responder (NR).

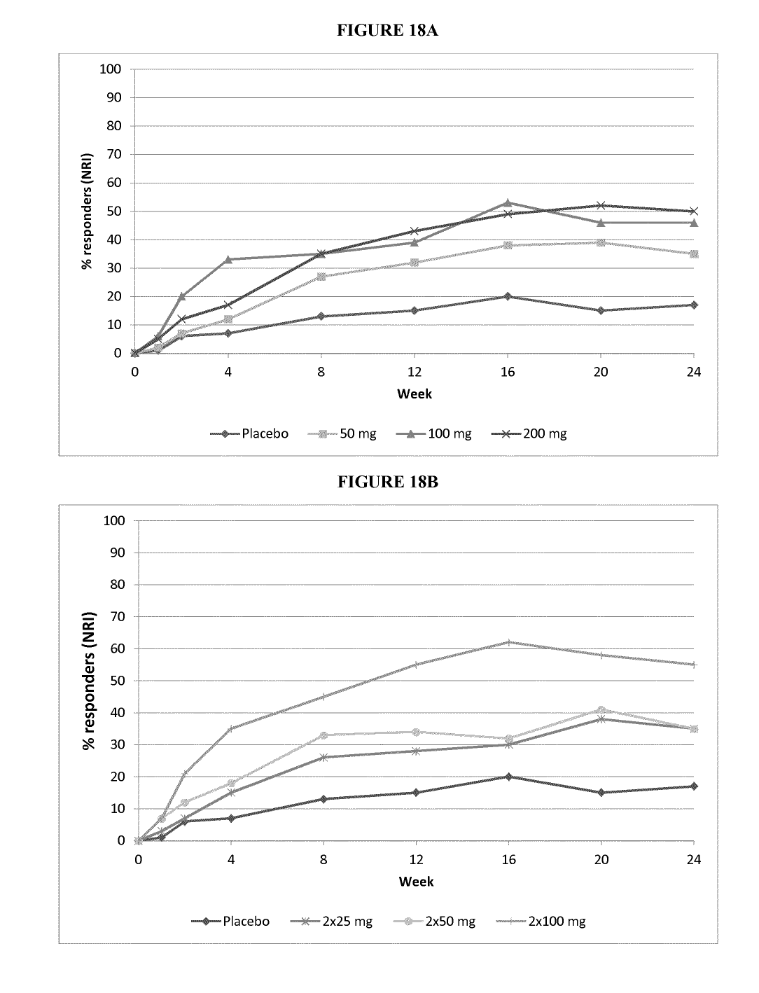

FIG. 19 shows the percentage of the patient population achieving an ACR70 response at the 1, 2, 4, 8, 12, 16, 20 and 24 week treatment time points in patients in Study 1. The y-axis is the percentage of patients meeting the ACR70 criteria. At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. Subjects who switched treatment at week 12 were handled as if they discontinued at week 12 for the purpose of statistical analysis, whereas subjects in the other groups maintained their randomized treatment until Week 24. Consequently, the data reported from week 16 to week 24 only refers to the data for the subjects continuing on the same treatment course from week 0 to week 24. FIG. 19A shows: placebo (diamonds), 50 mg q.d. (squares), 100 mg q.d. (triangles) or 200 mg q.d. (tilted crosses). FIG. 19B shows: placebo (diamonds), 25 mg b.i.d. (asterisk), 50 mg b.i.d. (circles) or 100 mg b.i.d. (vertical crosses). The results shown in the figures are calculated using the non-responder imputation (NRI), which assigns a value of nonresponse to missing data points, i.e. any patient who drops out is assumed to be a non-responder (NR).

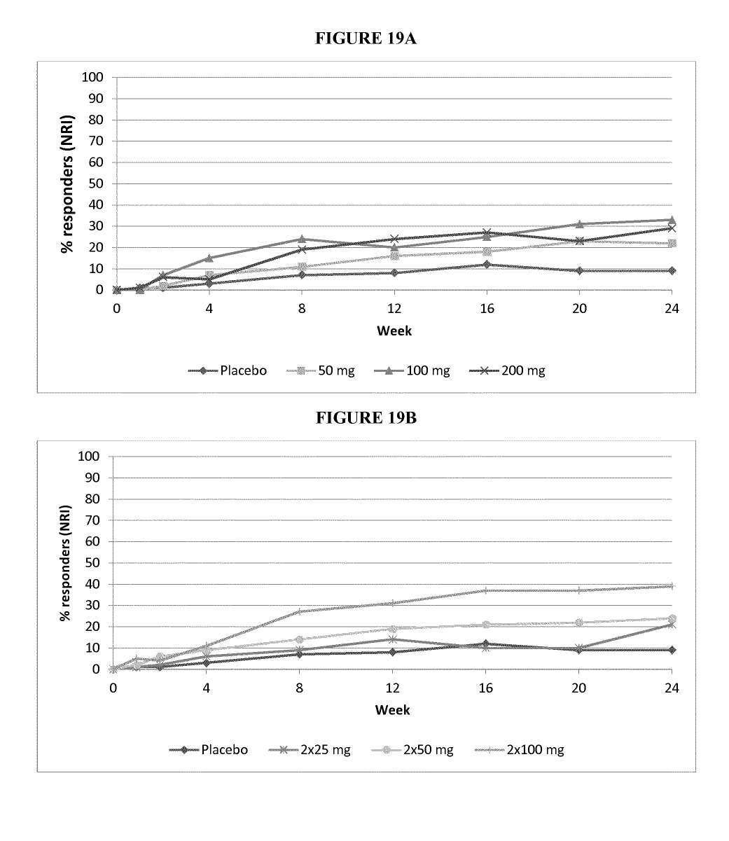

FIG. 20 shows the decrease in the DAS28(CRP) score in the patient population at the 1, 2, 4, 8, 12, 16, 20 and 24 week time points for each dose in Study 1. At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. Subjects who switched treatment at week 12 were handled as if they discontinued at week 12 for the purpose of statistical analysis, whereas subjects in the other groups maintained their randomized treatment until Week 24. Consequently, the data reported from week 16 to week 24 only refers to the data for the subjects continuing on the same treatment course from week 0 to week 24. FIG. 20A shows placebo (diamonds), 50 mg q.d. (squares), 100 mg q.d. (triangles) or 200 mg q.d. (tilted crosses). FIG. 20B shows: placebo (diamonds), 25 mg b.i.d. (asterisk), 50 mg b.i.d. (circles) or 100 mg b.i.d. (vertical crosses). The results shown in the figures are calculated using Last-Observation-Carried-Forward (LOCF), which handles missing data by assigning the value recorded at the patient's last visit to all subsequent missed visits.

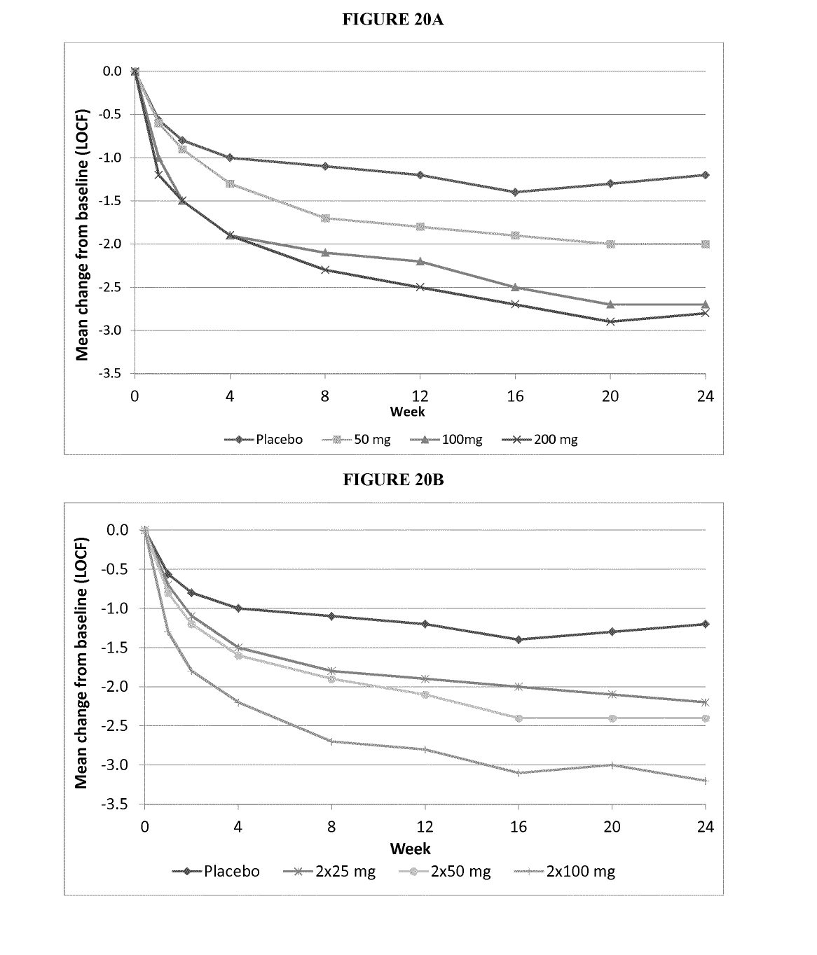

FIG. 21 shows the mean change in the haemoglobin levels in g/L in the patient population at the 1, 2, 4, 8, 12, 16, 20 and 24 week time points for each dose in Study 1. At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. FIG. 21A shows 25 mg b.i.d. (2×25 mg in resp, asterisk), 50 mg b.i.d. (2×50 mg, filled circles), 50 mg q.d. (50 mg in resp, filled diamonds), 100 mg b.i.d. (2×100 mg, upward crosses), 100 mg q.d. (100 mg, filled triangles), 200 mg q.d. (200 mg, tilted crosses), vs placebo (Placebo in resp, filled squares). FIG. 21B shows the mean change for switched groups from placebo to 100 mg q.d. (filled triangles), from placebo to 50 mg b.i.d. (filled circles), from 50 mg q.d. to 100 mg q.d. (filled diamonds), from 25 mg b.i.d. to 50 mg b.i.d. (2×25 mg to 2×50mg, filled squares).

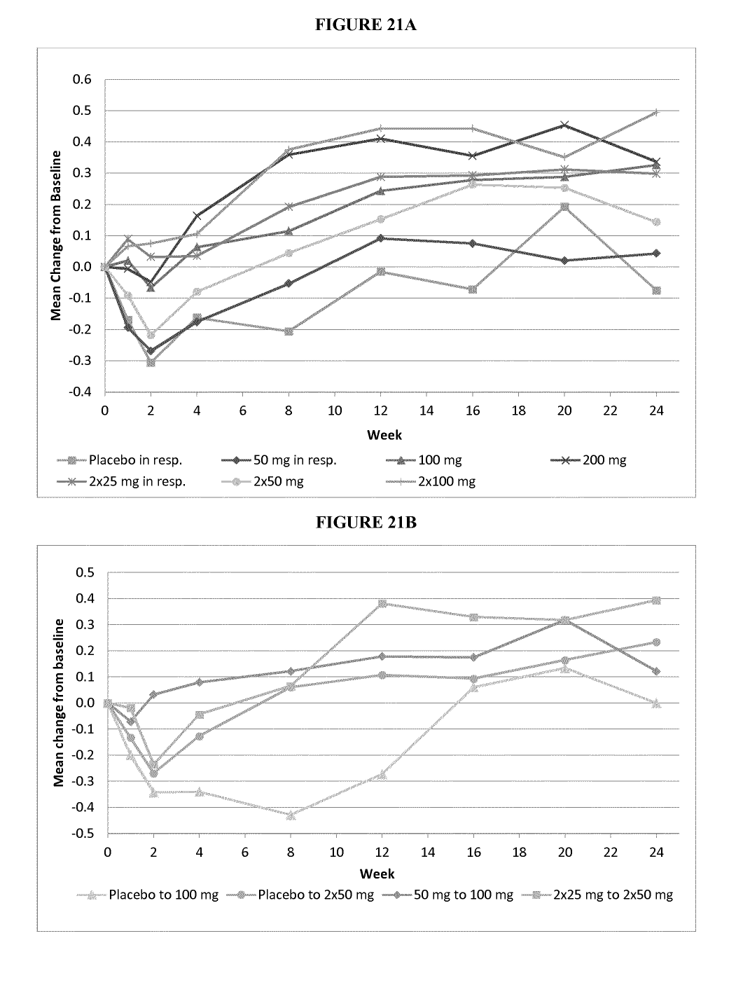

FIG. 22 shows the mean change in the number of neutrophils in GI/L in the patient population at the 1, 2, 4, 8, 12, 16, 20 and 24 week time points for each dose in Study 1. At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. FIG. 22A shows 25 mg b.i.d. (2×25mg in resp, asterisk), 50 mg b.i.d. (2×50 mg, filled circles), 50 mg q.d. (50 mg in resp, filled diamonds), 100 mg b.i.d. (2x100 mg, upward crosses), 100 mg q.d. (100 mg, filled triangles), 200 mg q.d. (200 mg, tilted crosses), vs placebo (Placebo in resp, filled squares). FIG. 22B shows the mean change for switched groups from placebo to 100 mg q.d. (filled triangles), from placebo to 50 mg b.i.d. (filled circles), from 50 mg q.d. to 100 mg q.d. (filled diamonds), from 25 mg b.i.d. to 50 mg b.i.d. (2×25 mg to 2×50 mg, filled squares).

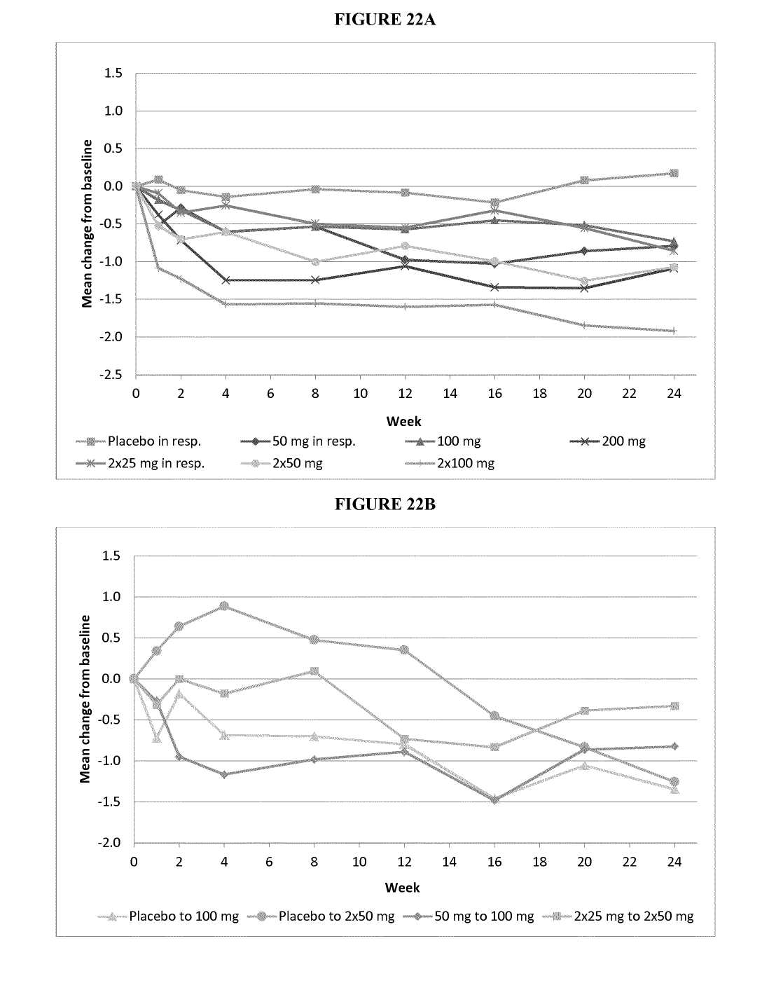

FIG. 23 shows the percentage of the patient population achieving an ACR20 response at the 1, 2, 4, 8, 12, 16, 20 and 24 week treatment time points in Study 2. At Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24. Consequently, at week 12, there are no subjects on placebo, and only the responding patients continuing for the entire 24 weeks on their initial treatment course (50 mg q.d., 100 mg q.d., and 200 mg q.d.) are reported. The y-axis is the percentage of patients meeting the ACR20 criteria. FIG. 23 shows the following groups: a) placebo (filled diamonds), b) 50 mg q.d. (filled squares), c) 100 mg q.d. (filled triangles) and d) 200 mg q.d. (tilted crosses). The results shown in the figures are calculated using the non-responder imputation (NRI), which assigns a value of nonresponse to missing data points, i.e. any patient who drops out is assumed to be a non-responder (NR).

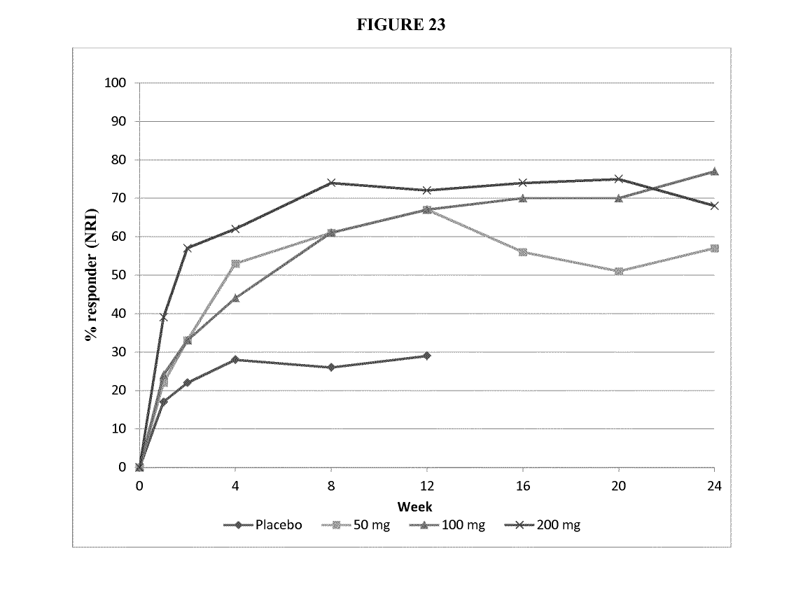

FIG. 24 shows the percentage of the patient population achieving an ACR50 response at the 1, 2, 4, 8, 12, 16, 20 and 24 week treatment time points in Study 2. At Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24. Consequently, at week 12, there are no subjects on placebo, and only the responding patients continuing for the entire 24 weeks on their initial treatment course (50 mg q.d., 100 mg q.d., and 200 mg q.d.) are reported. The y-axis is the percentage of patients meeting the ACR50 criteria. FIG. 24 shows the following groups: a) placebo (filled diamonds), b) 50 mg q.d. (filled squares), c) 100 mg q.d. (filled triangles) and d) 200 mg q.d. (tilted crosses). The results shown in the figures are calculated using the non-responder imputation (NRI), which assigns a value of nonresponse to missing data points, i.e. any patient who drops out is assumed to be a non-responder (NR).

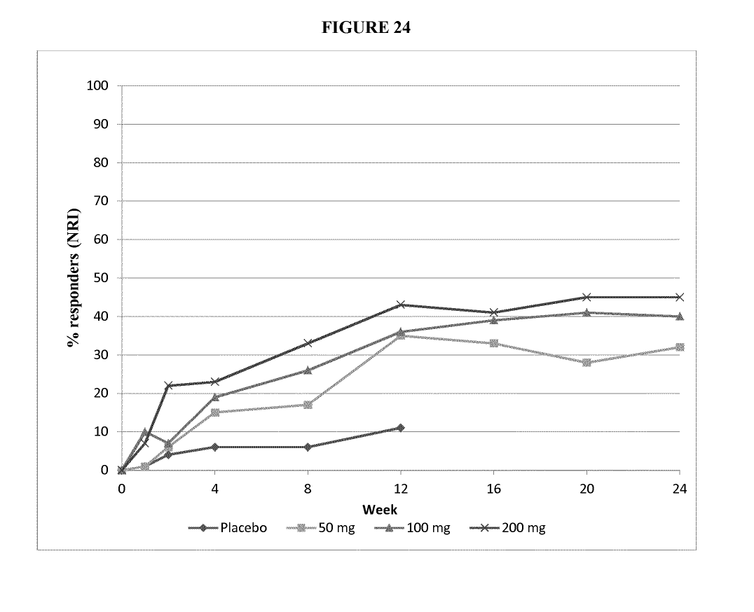

FIG. 25 shows the percentage of the patient population achieving an ACR70 response at the 1, 2, 4, 8, 12, 16, 20 and 24 week treatment time points in patients in Study 2. At Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24. Consequently, at week 12, there are no subjects on placebo, and only the responding patients continuing for the entire 24 weeks on their initial treatment course (50 mg q.d., 100 mg q.d., and 200 mg q.d.) are reported.The y-axis is the percentage of patients meeting the ACR70 criteria. FIG. 25 shows the following groups: a) placebo (filled diamonds), b) 50 mg q.d. (filled squares), c) 100 mg q.d. (filled triangles) and d) 200 mg q.d. (tilted crosses). The results shown in the figures are calculated using the non-responder imputation (NRI), which assigns a value of nonresponse to missing data points, i.e. any patient who drops out is assumed to be a non-responder (NR).

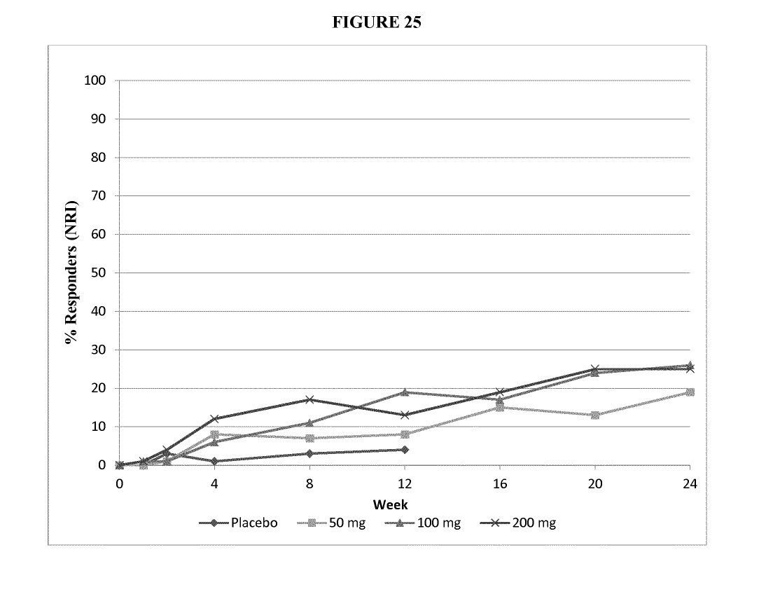

FIG. 26 shows the decrease in the DAS28(CRP) score in the patient population at the 1, 2, 4, 8, 12, 16, 20 and 24 week time points for each dose in Study 2. At Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24. Consequently, at week 12, there are no subjects on placebo, and only the responding patients continuing for the entire 24 weeks on their initial treatment course (50 mg q.d., 100 mg q.d., and 200 mg q.d.) are reported. FIG. 26 shows the following groups: a) placebo (filled diamonds), b) 50 mg q.d. (filled squares), c) 100 mg q.d. (filled triangles) and d) 200 mg q.d. (tilted crosses). The results shown in the figures are calculated using Last-Observation-Carried-Forward (LOCF), which handles missing data by assigning the value recorded at the patient's last visit to all subsequent missed visits.

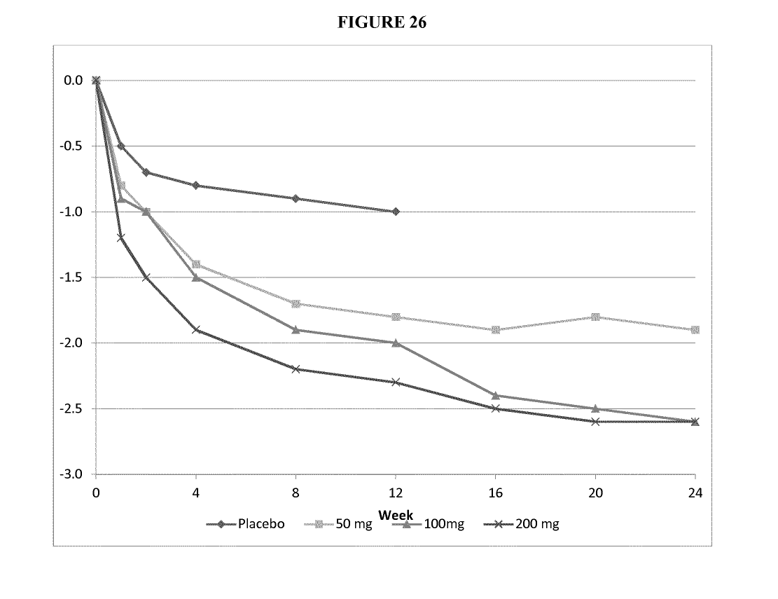

FIG. 27 shows the mean change in the haemoglobin levels in g/L in the patient population at the 1, 2, 4, 8, 12, 16, 20 and 24 week time points for each dose in Study 2. At Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24. FIG. 27 shows the following groups: a) placebo switching to 100 mg q;d. at week 12 (filled diamonds), b) continued 50 mg q.d. (filled triangles), c) non-responders switching from 50 mg q.d. to 100 mg q.d. at week 12 (tilted cross), d) continued 100 mg q.d. (filled circles) and d) continued 200 mg q.d. (asterisk).

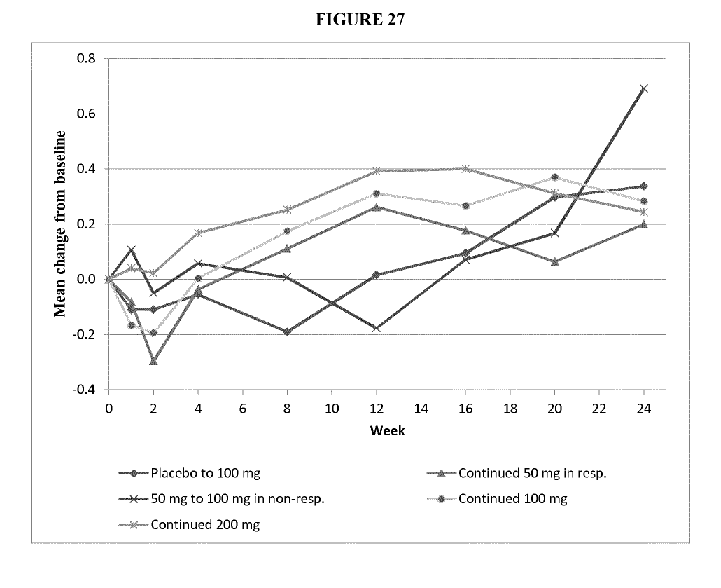

FIG. 28 shows the mean change in the number of neutrophils in GI/L in the patient population at the 1, 2, 4, 8, 12, 16, 20 and 24 week time points for each dose in Study 2. At Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24. FIG. 28 shows the following groups: a) placebo switching to 100 mg q;d. at week 12 (filled diamonds), b) continued 50 mg q.d. (filled triangles), c) non-responders switching from 50 mg q.d. to 100 mg q.d. at week 12 (tilted cross), d) continued 100 mg q.d. (filled circles) and d) continued 200 mg q.d. (asterisk).

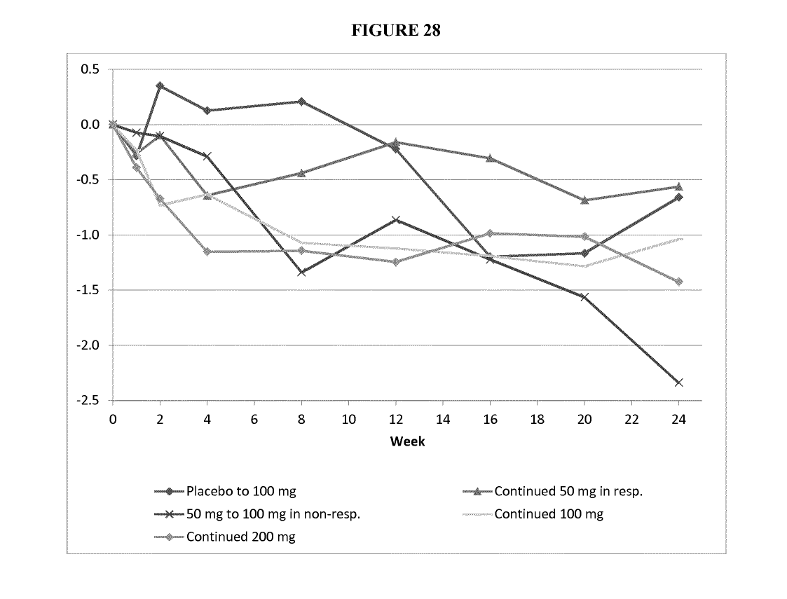

FIG. 29 shows the decrease in the serum CRP level in mg/L in the patient population at the 1, 2, 4, 8, 12, 16, 20 and 24 week time points for each dose in Study 1. FIG. 29A shows placebo (filled diamonds), 50 mg q.d. (filled squares), 100 mg q.d. (filled triangles) or 200 mg q.d. (tilted crosses). FIG. 29B shows: placebo (filled diamonds), 25 mg b.i.d. (asterisks), 50 mg b.i.d. (filled circles) or 100 mg b.i.d. (crosses). The results shown in the figures are calculated using Last-Observation-Carried-Forward (LOCF), which handles missing data by assigning the value recorded at the patient's last visit to all subsequent missed visits.

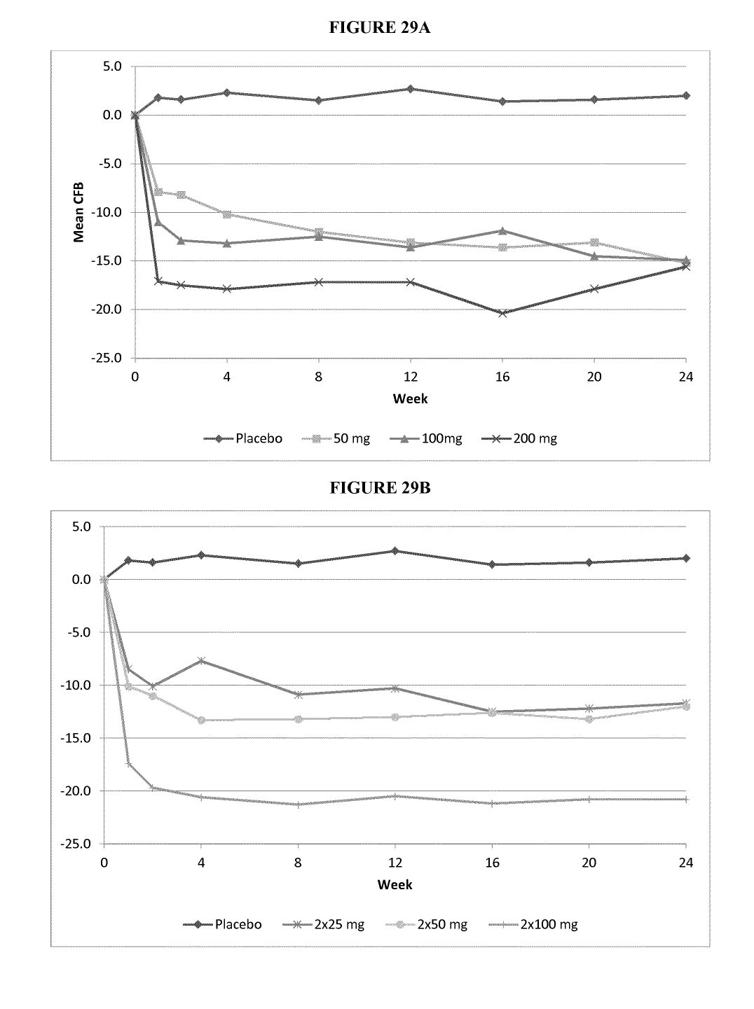

FIG. 30 shows the decrease in the Subject Global Assessment using a Visual Analog Scale (VAS), which is performed as part of the panel of ACR measurements, in the patient population at the 1, 2, 4, 8 and 12 week time points for each dose in Study 1. The scale is 100 mm, the decrease in mm is shown. FIG. 30A shows placebo (filled diamonds), 50 mg q.d. (filled squares), 100 mg q.d. (filled triangles) or 200 mg q.d. (tilted crosses). FIG. 30B shows: placebo (filled diamonds), 25 mg b.i.d. (asterisks), 50 mg b.i.d. (filled circles) or 100 mg b.i.d. (crosses). The results shown in the figures are calculated using Last-Observation-Carried-Forward (LOCF), which handles missing data by assigning the value recorded at the patient's last visit to all subsequent missed visits.

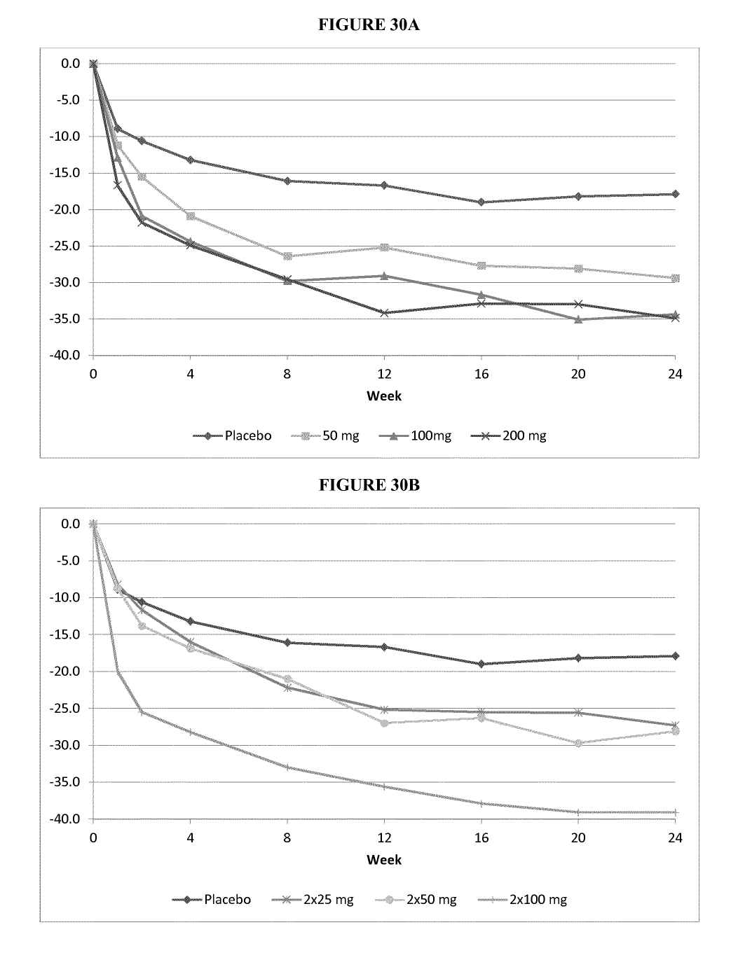

FIG. 31: shows the patient distribution throughout Study 1 over the 24 weeks. From week 0 to 12, the patients were randomized and distributed within the following groups: a) placebo, b) 50 mg q.d., c) 100 mg q.d., d) 200 mg q.d., e) 25 mg b.i.d., f) 50 mg b.i.d., and 100 mg b.i.d. At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. Subjects who switched treatment at week 12 were handled as if they discontinued at week 12 for the purpose of statistical analysis, whereas subjects in the other groups maintained their randomized treatment until Week 24

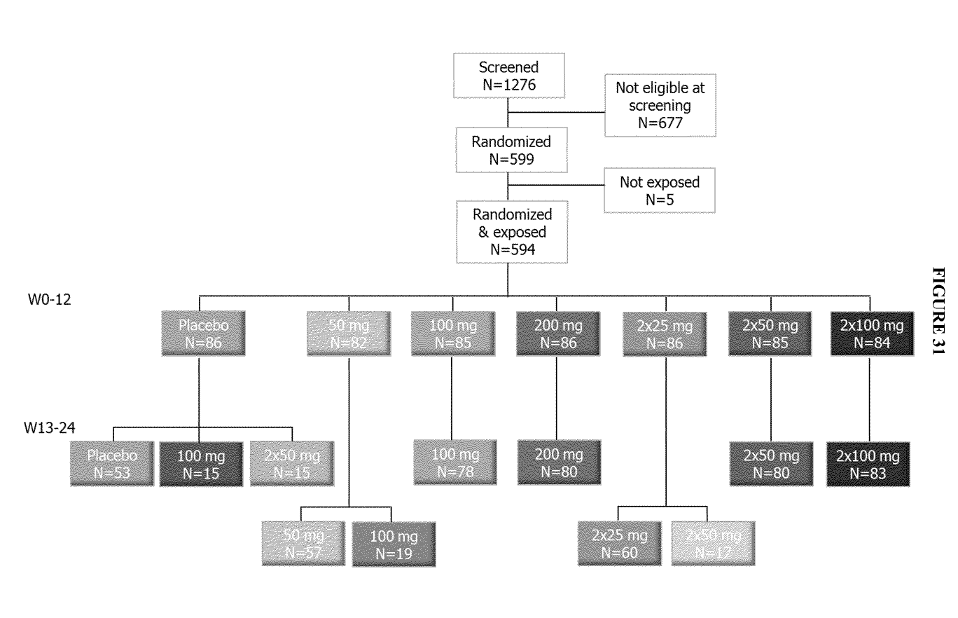

FIG. 32: shows the patient distribution throughout Study 2 over the 24 weeks. From week 0 to 12, the patients were randomized and distributed within the following groups: a) placebo, b) 50 mg q.d., c) 100 mg q.d., and d) 200 mg q.d. At Week 12, all subjects on placebo and the subjects on the 50 mg dose who did not achieve at least a 20% improvement in swollen joint count (SJC66) and tender joint count (TJC68) were assigned to 100 mg q.d. in a blinded fashion and continued treatment until Week 24. Subjects in the other groups maintained their randomized treatment until Week 24.

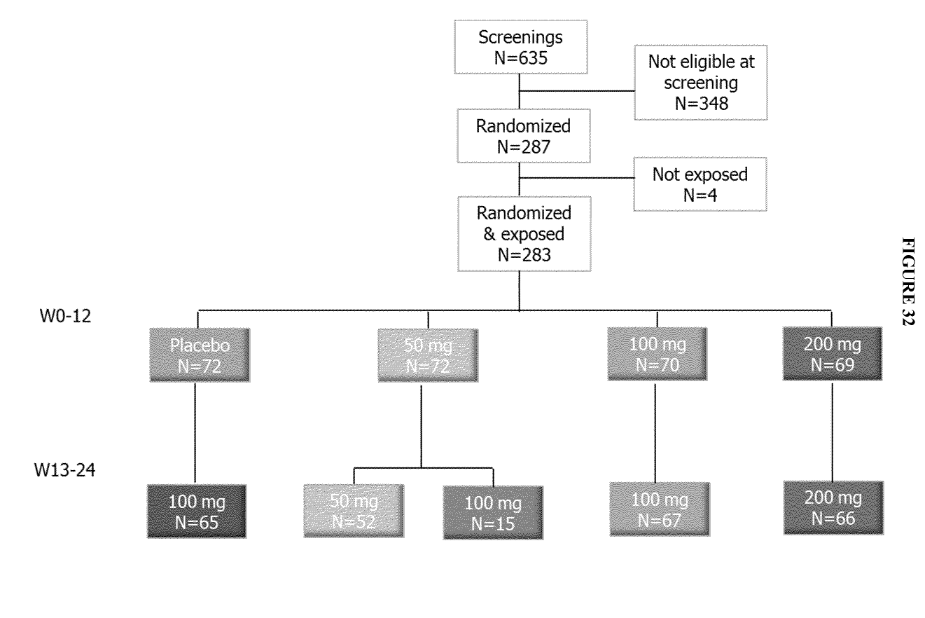

FIG. 33 shows the patient distribution throughout Study 3 over 10 weeks. The patients were randomized and distributed within the following groups a) placebo, and b) 200 mg q.d. over 10 weeks.

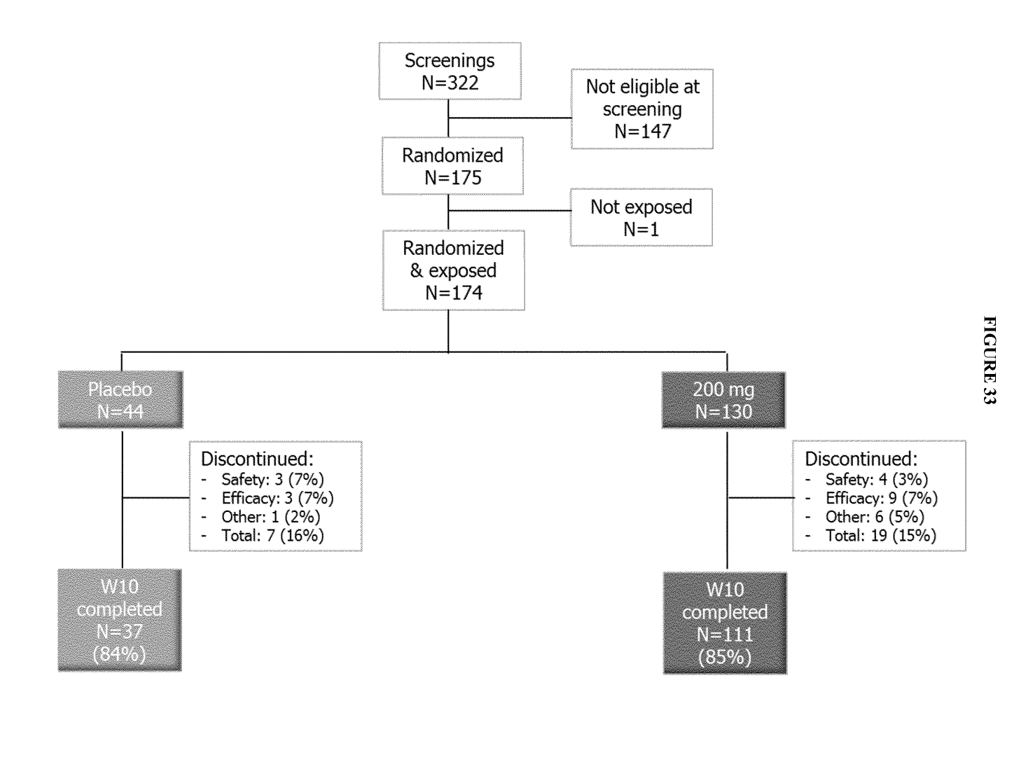

FIG. 34 Shows the patient distribution throughout Study 5 over 20 weeks. From week 0 to 10, the patients were randomized and distributed within the following groups: a) placebo, and b) 200 mg q.d. at week 10, patients are categorized as responders and non-responders. From the week 10 to week 20, a) the initial placebo responders are kept on placebo, and b) the initial placebo non-responders are put on a 100 mg q.d. dose regimen. In the initial 200 mg q.d. group, the responders are randomized between c) placebo, d) 100 mg q.d., e) 200 mg q.d. until week 20, whereas the non-responders are randomized between f) placebo, and g) 200 mg q.d. dose until week 20.

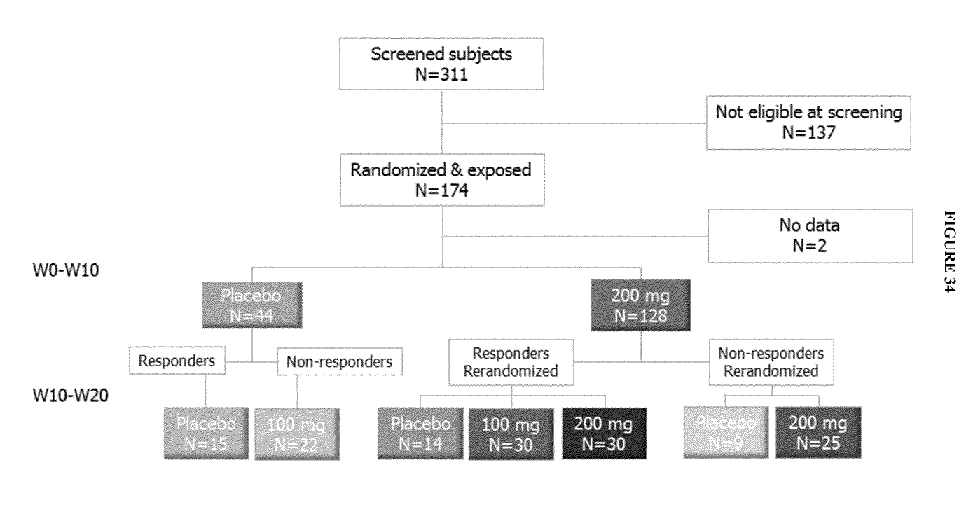

FIG. 35 shows the % responders achieving clinical remission (CDAI score <150 points) from week 10 to week 20 in the initial (from week 0 to week 10) 200 mg responders group, randomized into the groups a) 200 mg responders switching to placebo (filled diamonds), b) 200 mg responders switching to 100 mg (tilted crosses), and 200 mg responders continued on 200 mg (filled squares). FIG. 35A shows the NRI imputation; whereas FIG. 35B shows the LOCF imputation.

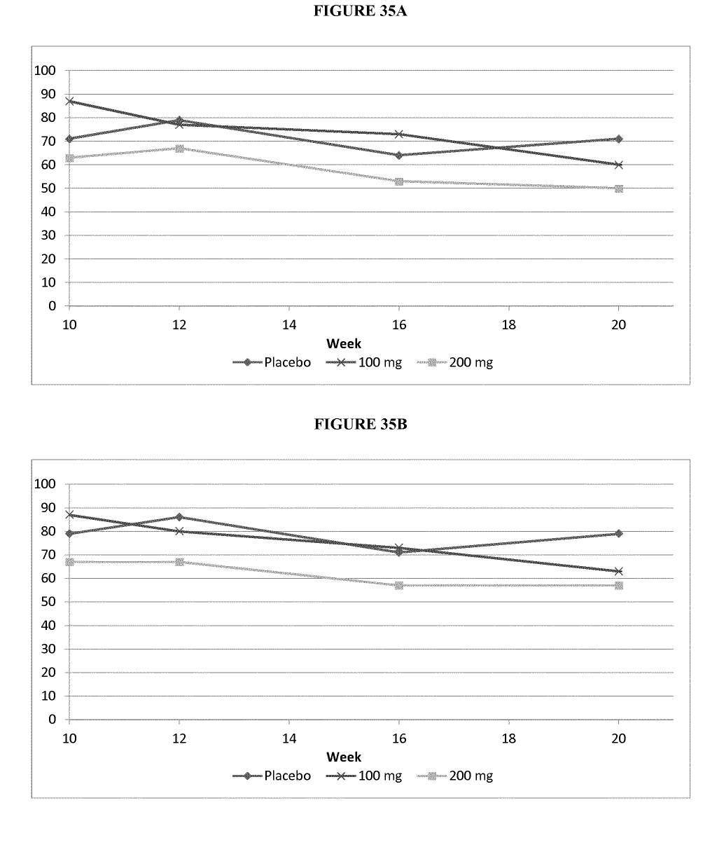

FIG. 36 shows the % responders achieving clinical remission (CDAI score <100 points) from week 10 to week 20 in the initial (from week 0 to week 10) 200 mg responders group, randomized into the groups a) 200 mg responders switching to placebo (filled diamonds), b) 200 mg responders switching to 100 mg (tilted crosses), and 200 mg responders continued on 200 mg (filled squares). FIG. 36A shows the NRI imputation; whereas FIG. 36B shows the LOCF imputation.

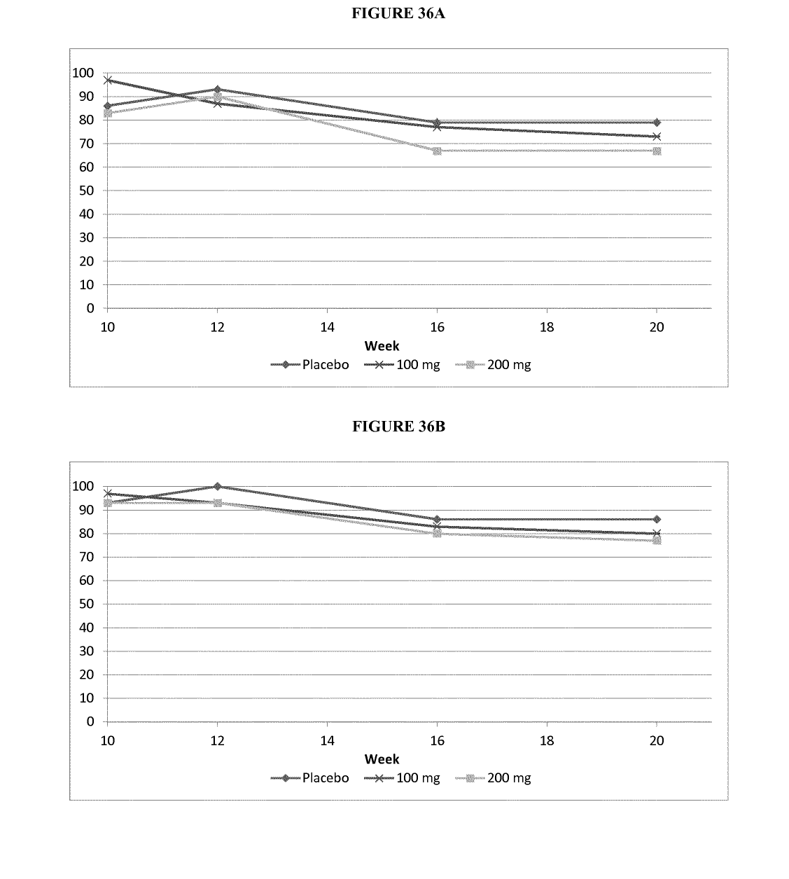

FIG. 37 shows the % responders achieving clinical remission (CDAI score <70 points) from week 10 to week 20 in the initial (from week 0 to week 10) 200 mg responders group, randomized into the groups a) 200 mg responders switching to placebo (filled diamonds), b) 200 mg responders switching to 100 mg (tilted crosses), and 200 mg responders continued on 200 mg (filled squares). FIG. 37A shows the NRI imputation; whereas FIG. 37B shows the LOCF imputation.

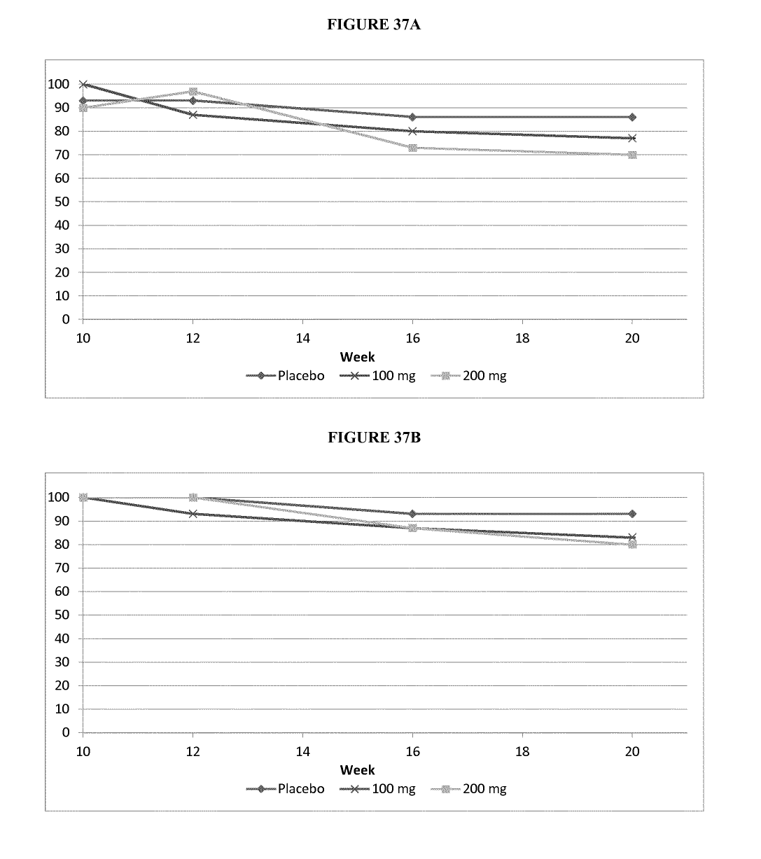

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

As used herein the term ‘inflammatory condition(s)’ refers to the group of conditions including, rheumatoid arthritis, osteoarthritis, juvenile idiopathic arthritis, psoriasis, psoriatic arthritis, allergic airway disease (e.g. asthma, rhinitis), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. Particularly the term refers to rheumatoid arthritis, osteoarthritis, allergic airway disease (e.g. asthma) and inflammatory bowel diseases.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Solvate’ refers to forms of the compound that are associated with a solvent, usually by a solvolysis reaction. This physical association includes hydrogen bonding. Conventional solvents include water, EtOH, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Effective amount’ means the amount of a compound of the invention that, when administered to a subject for treating a disease, is sufficient to effect such treatment for the disease. The “effective amount” can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term ‘inflammatory diseases’ refers to the group of conditions including, rheumatoid arthritis, osteoarthritis, juvenile idiopathic arthritis, psoriasis, psoriatic arthritis, allergic airway disease (e.g. asthma, rhinitis), chronic obstructive pulmonary disease (COPD), inflammatory bowel diseases (IBD) (e.g. Crohn's disease, Whipple, chronic ulcerative colitis, or colitis), endotoxin-driven disease states (e.g. complications after bypass surgery or chronic endotoxin states contributing to e.g. chronic cardiac failure), and related diseases involving cartilage, such as that of the joints. Particularly the term refers to rheumatoid arthritis, osteoarthritis, allergic airway disease (e.g. asthma), chronic obstructive pulmonary disease (COPD) and inflammatory bowel diseases. More particularly the term refers to rheumatoid arthritis, and inflammatory bowel diseases (IBD) (e.g. Crohn's disease, Whipple, chronic ulcerative colitis, or colitis).

As used herein a “patient who previously had an insufficient response to methotrexate therapy” means a patient where a Clinician has previous found that their response to the treatment did not achieve the expected levels. In a specific embodiment this means a patient who has not improved by three (3) months after the start of the treatment. In an alternative embodiment, this means a patient who has not reached the target of low disease activity or remission within six (6) months of starting treatment (Smolen et al., 2014)

The terms “ACR20”, “ACR50” and “ACR70” as used herein are scores which indicate how much a patient's rheumatoid arthritis has improved according to criteria set out by the American College of Rheumatology (ACR). The ACR score represents a percentage. An ACR20 score means that a person's RA has improved by 20%, an ACR50 score means it has improved by 50%, and an ACR70 score means it has improved by 70%. Specifically, to qualify for an ACR20 score, a person with RA must have at least 20% fewer tender joints and at least 20% fewer swollen joints. In addition the patient must show a 20% improvement in at least three of the following five areas: the person's overall (global) assessment of his or her own RA, the physician's global assessment of the person's RA, the person's assessment of his or her own pain, the person's assessment of his or her own physical functioning, and the results of an erythrocyte sedimentation rate or C-reactive protein (CRP) blood test (both of which test for inflammation). ACR50 and ACR70 scores use the same criteria but require 50% and 70% improvement, respectively, (Felson et al., 1993). In the data presented herein the CRP blood test is used.

As used herein, the term ‘DAS28(CRP)’ refers to a clinical scoring ranging from 2.0 to 10.0 to measure the progress and improvement of rheumatoid arthritis in a patient, and includes a 28 tender and swollen joint count, CRP measurement from blood analysis, and a general health assessment on a visual analog scale. A DAS28(CRP) value below 2.6 is indicative of remission, A DAS28(CRP) between 2.6 and 3.2 is indicative of low disease activity, between 3.2 and 5.1 is indicative of moderate disease activity, whereas a DAS28(CRP) above 5.1 is linked to high disease activity (Wells et al., 2009).

As used herein, the term ‘CRP’ refers to the C-Reactive protein in blood serum and is a marker of inflammation. In particular guidelines for CRP are widely available, and , and normal values of <0.5 mg/dL are recommended (Porter, 2011).

As used herein, the “Mayo Score” is a clinical scoring method to determine the severity of inflammatory bowel diseases (IBD) such as Crohn's disease and ulcerative colitis. It is composed of four categories (bleeding, stool frequency, physician assessment, and endoscopic appearance) each of which is rated from 0-3, the four scores are then summed to give a total score that ranges from 0-12.

“Crohn's Disease Activity Index (CDAI)” is a clinical scoring methods used to determine the severity of Crohn's disease, which is made up of a number of items which are then multiplied by a weighting factor to give a final score. The items included are: number of liquid or very soft stools, abdominal pain, general well-being, extra-intestinal manifestations of Crohn's Disease, use of Lomotil/Imodium/ opiates for diarrhea, abdominal mass, hematocrit (%) and body weight (Freeman, 2008).

“Ulcerative colitis (UC) disease activity index (UC DAI)” is a clinical scoring method used to determine the severity of Ulcerative colitis. The index assesses four variables, which include stool frequency, severity of bleeding, colonic mucosal appearance, and the physician's overall assessment of disease activity. Each variable is scored from 0-3 so that the total index score ranges from 0-12; 0-2: remission; 3-6: mild; 7-10: moderate; >10: severe UC (Torsi et al., 2010).

As used herein the term ‘TNF-naïve patient’ refers to a patient previously not exposed to anti-TNF monoclonal antibody treatment or subjects previously exposed to anti-TNF therapy (for example and without limitation infliximab, golimumab, adalimumab, certolizumab and/or certolizumab pegol) at a dose registered for the treatment of CD that has been discontinued at least 8 weeks prior to entering the study.

As used herein the term ‘TNF-experienced patient’ refers to a patient that is receiving at the time of entering the study or has received anti-TNF monoclonal antibody treatment (for example and without limitation infliximab, golimumab, adalimumab, certolizumab and/or certolizumab pegol) and is no longer responsive to such treatment.

As used herein the term ‘anti-TNF pharmaceutical’ refers a class of drugs that are used to treat inflammatory conditions, in particular rheumatoid arthritis (RA), psoriatic arthritis, juvenile arthritis, inflammatory bowel disease (Crohn's and ulcerative colitis), ankylosing spondylitis and psoriasis. TNF is a chemical produced by the immune system that causes inflammation in the body. In healthy individuals, excess TNF in the blood is blocked naturally, but in those inflammatory conditions, higher levels of TNF in the blood lead to more inflammation and persistent symptoms. Particular examples of anti-TNF pharmaceutical include infliximab, golimumab, adalimumab, certolizumab and certolizumab pegol.

As used herein the term ‘corticosteroid’ or ‘glucocorticoid’ refers to pharmaceutical agents that act by downregulating the transcription of proinflammatory genes (e.g., NF-κB) involved in cytokine production. Particular examples of corticosteroids include hydrocortisone, methylprednisolone, prednisone, prednisolone, or budesonide.

As used herein, the term ‘prednisolone equivalent’ refers to a dose measurement method of a corticosteroid, and to the dose required of to achieve the same effect as the effect obtained with prednisone. As a way of example 20 mg prednisone equivalent refers to the dose of corticosteroid administered to achieve the same effect as a dose of 20 mg of prednisone in a given patient. (Daley-Yates, P. T., 2015. Inhaled corticosteroids: potency, dose equivalence and therapeutic index. Br. J. Clin. Pharmacol. 80, 372-380. doi:10.1111/bcp.12637).

The term “compound of the invention” and equivalent expressions, is meant to embrace the compound of the Formula I as herein described, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitro (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵0 and ¹³N, and would be useful in Positron Emission Topography (PET) studies for examining substrate receptor occupancy.

### The Invention

The present invention provides the compound of the invention for use in the treatment of inflammatory conditions, wherein said compound of the invention is according to Formula (I):

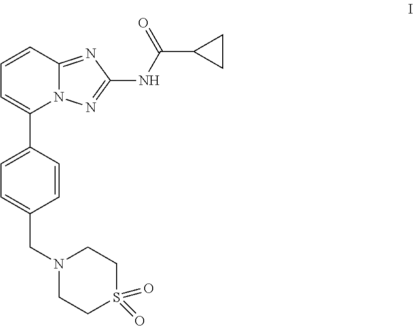 [SMILES: O=C(Nc1nc2cccc(-c3ccc(CN4CCS(=O)(=O)CC4)cc3)n2n1)C1CC1]

In one embodiment, the compound of the invention is a metabolite of the compound according to Formula I, said metabolite being according to Formula II:

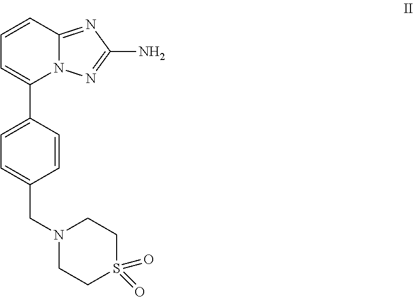 [SMILES: Nc1nc2cccc(-c3ccc(CN4CCS(=O)(=O)CC4)cc3)n2n1]

In one embodiment a compound of the invention is not an isotopic variant.

In one aspect a compound of the invention according to any one of the embodiments herein described is present as the free base.

In one aspect a compound of the invention according to any one of the embodiments herein described is a pharmaceutically acceptable salt.

In one aspect a compound of the invention according to any one of the embodiments herein described is a solvate of the compound.

In one aspect a compound of the invention according to any one of the embodiments herein described is a solvate of a pharmaceutically acceptable salt of a compound. In a particular embodiment, the solvate of a pharmaceutically acceptable salt is a [Compound according to Formula I:HCl:3H₂O] adduct.

It will be appreciated that compounds of the invention may be metabolized to yield biologically active metabolites.

In one embodiment, the present invention provides the compound of the invention or pharmaceutical compositions comprising the compound of the invention, for use in the treatment of inflammatory conditions when dosed orally at a dose of between 25 mg to 400 mg, administered once or twice a day. In particular, the compound of the invention is dosed orally at a dose of between 100 mg to 250 mg. In particular, the dose is selected from 25 mg twice per day (b.i.d.), 50 mg once a day (q.d.), 50 mg b.i.d., 100 mg q.d., 100 mg b.i.d., and 200 mg q.d. In particular, the compound of the invention is for use in the treatment of rheumatoid arthritis or IBD (e.g. Crohn's disease or ulcerative colitis).

In one embodiment, the present invention provides the compound of the invention or a pharmaceutical compositions comprising the compound of the invention, for use in the treatment of inflammatory conditions when dosed orally at a dose of between 25 mg to 400 mg, administered once or twice a day. In particular, the compound of the invention is dosed orally at a dose of between 100 mg to 250 mg. In particular, the dose is selected from at a dose selected from 25 mg twice per day (b.i.d.), 50 mg once a day (q.d.), 50 mg b.i.d., 100 mg q.d., 100 mg b.i.d., and 200 mg q.d. in patients who have previously had an insufficient response to methotrexate. In particular, the compound of the invention is for use in the treatment of rheumatoid arthritis or IBD (e.g. Crohn's disease or ulcerative colitis).

In another embodiment, the present invention provides the compound of the invention, or a pharmaceutical compositions comprising the compound of the invention for use in the manufacture of a medicament for use in the treatment of inflammatory conditions, wherein said medicament provides a dose to the patient of between 25-400 mg once or twice per day. In particular, said medicament is dosed orally at a dose of between 100 mg to 250 mg. In particular, said medicament provides a dose selected from 25 mg twice per day (b.i.d.), 50 mg once a day (q.d.), 50 mg b.i.d., 100 mg q.d., 100 mg b.i.d., and 200 mg q.d. In particular, the compound of the invention is for use in the treatment of rheumatoid arthritis or IBD (e.g. Crohn's disease or ulcerative colitis).

In another embodiment, the present invention provides compounds of the invention, or pharmaceutical compositions comprising a compound of the invention for use in the manufacture of a medicament for use in the treatment of inflammatory conditions, wherein said medicament provides a dose to the patient of between 25-400 mg once or twice per day. In particular, said medicament is dosed orally at a dose of between 100 mg to 250 mg. In particular, said medicament provides a dose selected from 25 mg twice per day (b.i.d.), 50 mg once a day (q.d.), 50 mg b.i.d., 100 mg q.d., 100 mg b.i.d., and 200 mg q.d. in patients who have previously had an insufficient response to methotrexate. In particular, the compound of the invention is for use in the treatment of rheumatoid arthritis or IBD (e.g. Crohn's disease or ulcerative colitis).

In additional method of treatment aspects, this invention provides methods of treatment of a patient afflicted with an inflammatory condition, which methods comprise the administration of an effective amount of the compound of the invention or one or more of the pharmaceutical compositions herein described such that the patient receives a dose of between 25-400 mg once or twice per day. In particular, the compound of the invention is dosed orally at a dose of between 100 mg to 250 mg. In particular, the dose is selected from 25 mg twice per day (b.i.d.), 50 mg once a day (q.d.), 50 mg b.i.d., 100 mg q.d., 100 mg b.i.d., and 200 mg q.d. In particular, the compound of the invention is for use in the treatment of rheumatoid arthritis or IBD (e.g. Crohn's disease or ulcerative colitis).

In additional method of treatment aspects, this invention provides methods of treatment of a patient afflicted with an inflammatory condition, which methods comprise the administration of an effective amount of the compound of the invention or one or more of the pharmaceutical compositions herein described such that the patient receives a dose of between 25-400 mg once or twice per day. In particular, the compound of the invention is dosed orally at a dose of between 100 mg to 250 mg. In particular, the dose is selected from 25 mg twice per day (b.i.d.), 50 mg once a day (q.d.), 50 mg b.i.d., 100 mg q.d., 100 mg b.i.d., and 200 mg q.d. and where said patient previously had an insufficient response to methotrexate therapy. In particular, the compound of the invention is for use in the treatment of rheumatoid arthritis or IBD (e.g. Crohn's disease or ulcerative colitis).

In one embodiment, the present invention provides the compound of the invention or pharmaceutical compositions comprising the compound of the invention, for use in the treatment of IBD wherein the compound is first administered at an induction dose selected from 100 mg twice per day (b.i.d.), or 200 mg once a day (q.d.) for a period of 4-12 weeks, followed by a maintenance dose selected from 50 mg b.i.d., 100 mg q.d., 100 mg b.i.d., and 200 mg q.d. for a period of at least 4 weeks. In a particular embodiment the induction dose is administered for 8-12 weeks, in particular 10 weeks. In a specific embodiment the compound of the invention is administered at an induction dose of 200 mg q.d. for 10 weeks, followed by a maintenance dose of 100 mg q.d. or 200 mg q.d. for at least 4 weeks. In a particular embodiment, the IBD is selected from Crohn's disease or ulcerative colitis. In a specific embodiment the IBD is Crohn's disease.

In another embodiment, the present invention provides the compound of the invention, or a pharmaceutical compositions comprising the compound of the invention for use in the manufacture of a medicament for use in the treatment of IBD wherein the compound is first administered at an induction dose selected from 100 mg twice per day (b.i.d.), or 200 mg once a day (q.d.) for a period of 4-12 weeks, followed by a maintenance dose selected from 50 mg b.i.d., 100 mg q.d., 100 mg b.i.d., and 200 mg q.d. for a period of at least 4 weeks. In a particular embodiment the induction dose is administered for 8-12 weeks, in particular 10 weeks. In a specific embodiment the compound of the invention is administered at an induction dose of 200 mg q.d. for 10 weeks, followed by a maintenance dose of 100 mg q.d. or 200 mg q.d. for at least 4 weeks. In a particular embodiment, the IBD is selected from Crohn's disease or ulcerative colitis. In a specific embodiment the IBD is Crohn's disease.

In additional method of treatment aspects, this invention provides methods of treatment of a patient afflicted with an IBD, which methods comprise the administration of an effective amount of the compound of the invention or one or more of the pharmaceutical compositions herein described such that the patient receives an induction dose selected from 100 mg twice per day (b.i.d.), or 200 mg once a day (q.d.) for a period of 4-12 weeks, followed by a maintenance dose selected from 50 mg b.i.d., 100 mg q.d., 100 mg b.i.d., and 200 mg q.d. for a period of at least 4 weeks. In a particular embodiment the induction dose is administered for 8-12 weeks, in particular 10 weeks. In a specific embodiment the patient receives an induction dose of 200 mg q.d. for 10 weeks, followed by a maintenance dose of 100 mg q.d. or 200mg q.d. for at least 4 weeks. In a particular embodiment, the IBD is selected from Crohn's disease or ulcerative colitis. In a specific embodiment the IBD is Crohn's disease.

In one embodiment, with respect to the uses and methods described above, the patients are concomitantly dosed with methotrexate, in particular they receive between 7.5-25 mg once per week of methotrexate, in particular the patients receive 10-25 mg once per week of methotrexate.

In an alternative embodiment, with respect to the uses and methods described above, the patients are not concomitantly treated with methotrexate.

In an alternative embodiment, with respect to the uses and methods described above, the patients do not receive any additional treatments for rheumatoid arthritis or IBD (e.g. Crohn's disease or ulcerative colitis) concomitantly with the administration of the compound of the invention.

In a specific embodiment with respect to the uses and methods described above in rheumatoid arthritis patients, an ACR20 response is seen in at least 50% of the patient population after 4 weeks of treatment. Particularly, an ACR20 response is seen in at least 51%, at least 52%, at least 53%, at least 54%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94% or at least 95% or the patient population after 4 weeks of treatment. Particularly, the ACR20 response is seen after 8 weeks of treatment. Particularly, the ACR20 response is seen after 12 weeks of treatment. Particularly, the ACR20 response is seen after 16 weeks of treatment. Particularly, the ACR20 response is seen after 20 weeks of treatment. Particularly, the ACR20 response is seen after 24 weeks of treatment.

In a specific embodiment with respect to the uses and methods described above in rheumatoid arthritis patients, an ACR50 response is seen in at least 30% of the patient population after 4 weeks of treatment. Particularly, an ACR50 response is seen in at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, or at least 75% or the patient population after 4 weeks of treatment. Particularly, the ACR50 response is seen after 8 weeks of treatment. Particularly, the ACR50 response is seen after 12 weeks of treatment. Particularly, the ACR50 response is seen after 16 weeks of treatment. Particularly, the ACR50 response is seen after 20 weeks of treatment. Particularly, the ACR50 response is seen after 24 weeks of treatment.

In a specific embodiment with respect to the uses and methods described above in rheumatoid arthritis patients, an ACR70 response is seen in at least 10% of the patient population after 4 weeks of treatment. Particularly, an ACR70 response is seen in at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, or at least 50% or the patient population after 4 weeks of treatment. Particularly, the ACR70 response is seen after 8 weeks of treatment. Particularly, the ACR70 response is seen after 12 weeks of treatment. Particularly, the ACR70 response is seen after 16 weeks of treatment. Particularly, the ACR70 response is seen after 20 weeks of treatment. Particularly, the ACR70 response is seen after 24 weeks of treatment.

It will be understood by a person of skill in the art, that improvements in the disease in patients may be identified, without needing to complete the full ACR or DAS28(CRP) panel of assessments. Therefore in a specific embodiment, with respect to the uses and methods described above in rheumatoid arthritis patients, a decrease in the Subject Global Assessment as measured using a Visual Analog Scale is seen in the patient. Particularly, a VAS of 100 mm is used, where a decrease of at least 20 mm is seen. It will be appreciated that this also is equivalent to a decrease of 20% if VAS of alternative lengths are used. Particularly, using a VAS of 100 mm a decrease of at least 25 mm, at least 30 mm, at least 35 mm or at least 40 mm is seen. These measurements can also be expressed as percentages if a VAS of an alternative length is used. Therefore in particular a decrease of at least 25%, at least 30%, or at least 40% is seen.

In a specific embodiment with respect to the uses and methods described above in rheumatoid arthritis patients, a decrease in the DAS28(CRP) score of at least 1.9 is seen in the patient. Particularly a decrease of at least 2.0, at least 2.1, at least 2.2, at least 2.3, at least 2.4, at least 2.5, at least 2.6, at least 2.7, at least 2.8, at least 2.9 or at least 3.0 is seen in the patient. In a specific embodiment, a decrease in the DAS28(CRP) score to a value of between 3.2-5.1 is seen (i.e. moderate disease activity),In a specific embodiment, a decrease in the DAS28(CRP) score to a value of between 2.61-3.19 is seen (i.e. low disease activity). In a specific embodiment, a decrease in the DAS28(CRP) score to a value of ≤2.6 is seen (i.e. remission). Particularly the decrease in the DAS28(CRP) score is seen after 4 weeks of treatment. Particularly, the decrease in the DAS28(CRP) score is seen after 8 weeks of treatment. Particularly the decrease in the DAS28(CRP) score is seen after 12 weeks of treatment. Particularly the decrease in the DAS28(CRP) score is seen after 16 weeks of treatment. Particularly the decrease in the DAS28(CRP) score is seen after 20 weeks of treatment. Particularly the decrease in the DAS28(CRP) score is seen after 24 weeks of treatment. More particularly, the decrease in the DAS28(CRP) score is seen after 8 weeks of treatment and maintained for at least a further 4 weeks. More particularly the decrease in the DAS28(CRP) score is seen after 8 weeks of treatment and maintained for at least a further 8 weeks. More particularly the decrease in the DAS28(CRP) score is seen after 8 weeks of treatment and is maintained for at least a further 12 weeks.

In a specific embodiment, with respect to the uses and methods described above, a decrease in the CRP levels in serum of at least 5 mg/L is seen in patients. Particularly a decrease in the CRP levels in the serum of at least 10 mg/L, at least 12.5 mg/L, at least 15 mg/L, at least 17.5 mg/L or at least 20 mg/L is seen in patients. In one embodiment, the decrease in the CRP levels is seen after 2 weeks of treatment, more particularly after 4 weeks of treatment, more particularly after 12 weeks of treatment. In a specific embodiment, a decrease of at least 15 mg/L is seen in patients administered the compound of the invention at 100 mg b.i.d. or 200 mg q.d.

In a specific embodiment, with respect to the uses and methods described above in IBD, a decrease in the Mayo score or the disease activity index (DAI) of at least 2 points is seen. More particularly a decrease of at least 3 points, at least 4 points, at least 5 points, at least 6 points, at least 7 points, at least 8 points, at least 9 points, at least 10 points, at least 11 points or of about 12 points is seen. In particular, the decrease is seen after 4 weeks of treatment. In particular the decrease is seen after 8 weeks of treatment. In particular the decrease is seen after 12 weeks of treatment. In particular, the decrease is seen after 16 weeks of treatment. In particular the decrease is seen after 20 weeks of treatment. In particular the decrease is seen after 24 weeks of treatment.

In a specific embodiment, with respect to the uses and methods described above in inflammatory conditions, an increase in the levels of haemoglobin are seen after 4 weeks of treatment. Particularly, an increase is seen after 8 weeks of treatment. Particularly an increase is seen after 12 weeks of treatment. In a specific embodiment an increase of at least 1 g/L is seen, particularly at least 1.5 g/L, at least 2 g/L, at least 2.5 g/L, at least 3 g/L or at least 3.5 g/L. In a specific embodiment an increase of at least 3 g/L is seen in patients administered the compound of the invention at a dose of either 100 mg b.i.d. or 200 mg q.d. In a specific embodiment, an increase of at least 3.5 g/L is seen in patients administered the compound of the invention at a dose of either 100 mg b.i.d. or 200 mg q.d. In a particular embodiment, said increase in haemoglobin levels is seen in patients who show an ACR20 response, more particularly in patients who show an ACR50 response, more particularly in patients who show an ACR70 response. In a specific embodiment, an increase of at least 2.5 g/L is seen in patients who show an ACR50 response, more particularly, an increase of at least 3 g/L or 3.5 g/L is seen in patients who show an ACR50 response. In a specific embodiment, an increase of at least 2.5 g/L is seen in patients who show an ACR70 response, more particularly, an increase of at least 3 g/L or 3.5 g/L is seen in patients who show an ACR70 response.

In another specific embodiment, with respect to the uses and methods described above in inflammatory conditions, an increase in the levels of haemoglobin are seen after 4 weeks of treatment. Particularly, an increase is seen after 8 weeks of treatment. Particularly an increase is seen after 12 weeks of treatment. Particularly an increase is seen after 16 weeks of treatment. Particularly an increase is seen after 20 weeks of treatment. Particularly an increase is seen after 24 weeks of treatment. In a specific embodiment an increase of at least 1 g/L is seen, particularly at least 1.5 g/L, at least 2 g/L, at least 2.5 g/L, at least 3 g/L, at least 3.5 g/L, at least 4.0 g/L, at least 4.5 g/L, or at least 5.0 g/L. In a specific embodiment an increase of at least 3 g/L is seen in patients administered the compound of the invention at a dose of either 100 mg b.i.d. or 200 mg q.d. In a specific embodiment, an increase of at least 3.5 g/L is seen in patients administered the compound of the invention at a dose of either 100 mg b.i.d. or 200 mg q.d. In a specific embodiment, an increase of at least 4.0 g/L is seen in patients administered the compound of the invention at a dose of either 100 mg b.i.d. or 200 mg q.d. In a specific embodiment, an increase of at least 4.5 g/L is seen in patients administered the compound of the invention at a dose of either 100 mg b.i.d. or 200 mg q.d. In a specific embodiment, an increase of at least 5.0 g/L is seen in patients administered the compound of the invention at a dose of either 100 mg b.i.d. or 200 mg q.d. In a particular embodiment, said increase in haemoglobin levels is seen in patients who show an ACR20 response, more particularly in patients who show an ACR50 response, more particularly in patients who show an ACR70 response. In a specific embodiment, an increase of at least 2.5 g/L is seen in patients who show an ACR50 response, more particularly, an increase of at least 3 g/L, 3.5 g/L, 4.0 g/L, 4.5 g/L, or 5.0 g/L is seen in patients who show an ACR50 response. In a specific embodiment, an increase of at least 2.5 g/L is seen in patients who show an ACR70 response, more particularly, an increase of at least 3 g/L, 3.5 g/L, 4.0 g/L, 4.5 g/L, or 5.0 g/L is seen in patients who show an ACR70 response.

In a specific embodiment, with respect to the uses and methods described above in inflammatory conditions, said increase in the levels of haemoglobin can be measured as a percentage change from baseline (CFB). In a specific embodiment an increase of at least 1% is seen, particularly at least 2%, at least 3% or at least 4%. In a specific embodiment an increase of at least 3% is seen in patients administered the compound of the invention at a dose of either 100 mg b.i.d. or 200 mg q.d. In a specific embodiment, an increase of at least 4% is seen in patients administered the compound of the invention at a dose of either 100 mg b.i.d. or 200 mg q.d. In a particular embodiment, said increase in haemoglobin levels is seen in patients who show an ACR20 response, more particularly in patients who show an ACR50 response, more particularly in patients who show an ACR70 response. In a specific embodiment, an increase of at least 2% is seen in patients who show an ACR50 response, more particularly, an increase of at least 3% or 4% is seen in patients who show an ACR50 response. In a specific embodiment, an increase of at least 2% is seen in patients who show an ACR70 response, more particularly, an increase of at least 3% or 4% is seen in patients who show an ACR70 response.

In a specific embodiment, with respect to the uses and methods described above in inflammatory conditions, said increase in the levels of haemoglobin can be measured as a percentage change from baseline (CFB). In a specific embodiment an increase of at least 1% is seen, particularly at least 2%, at least 3%, or at least 4%. In a specific embodiment an increase of at least 3% is seen in patients administered the compound of the invention at a dose of either 100 mg b.i.d. or 200 mg q.d. In a specific embodiment, an increase of at least 4% is seen in patients administered the compound of the invention at a dose of either 100 mg b.i.d. or 200 mg q.d. In a further specific embodiment, an increase of at least 4.3% is seen in patients administered the compound of the invention at a dose of either 100 mg b.i.d. or 200 mg q.d.In a particular embodiment, said increase in haemoglobin levels is seen in patients who show an ACR20 response, more particularly in patients who show an ACR50 response, more particularly in patients who show an ACR70 response. In a specific embodiment, an increase of at least 2% is seen in patients who show an ACR50 response, more particularly, an increase of at least 3%, 4% or 4.3% is seen in patients who show an ACR50 response. In a specific embodiment, an increase of at least 2% is seen in patients who show an ACR70 response, more particularly, an increase of at least 3%, 4% or 4.3% is seen in patients who show an ACR70 response.

In one embodiment, with respect to the uses and methods described above in inflammatory conditions, the increase in haemoglobin levels results in a reduced requirement to test the haemoglobin levels in the patients. Particularly, the frequency of haemoglobin testing may be reduced to no more than once per month, once per two month period, once per three month period or once per six month period.

In a further embodiment, with respect to the uses and methods described above, a decrease in the number of neutrophils is observed between the first treatment day and week 4, without a clinical presentation of neutropenia. In particular, a decrease of at least 0.5 GI/L in the number of neutrophils is seen between the first treatment day and week 4. In particular, a decrease of at least 1 GI/L, at least 1.1 GI/L, at least 1.2 GI/L, at least 1.3 GI/L, at least 1.4 GI/L or at least 1.5 GI/L is seen in the number of neutrophils between the baseline levels and week 4. In a particular embodiment, the decrease is maintained for at least 12 weeks from the first treatment day. In another particular embodiment, the decrease is maintained for at least 24 weeks from the first treatment day.

The levels of aspartate aminotransferase (AST) and/or alanine aminotransferase (ALT) are used as clinical biomarkers for liver health. Therefore, in one aspect in the uses and methods described above, the administration of the compound of the invention does not result in a clinically significant increase in AST and/or ALT levels, in particular the levels of AST and/or ALT are not increased more than 70% from the pre-treatment baseline levels. In a more particular embodiment the levels of AST and/or ALT are not increased more than 50% from the pre-treatment baseline levels. In a more particular embodiment, the levels of AST and/or ALT are not increased more than 25% from the pre-treatment baseline levels.

The levels of creatinine in serum or urine is used as a clinical biomarker for renal function. Therefore, in one aspect in the uses and methods described above the administration of the compound of the invention does not result in a clinically significant change in creatinine levels. In a particular embodiment after administration of the compound of the invention the creatinine levels are approximately 60-120 μmol/L in male patients, and approximately 55-100 μmol/L in female patients using Jaffe-based methods.

In a further embodiment, with respect to the uses and methods described above, the lymphocyte levels do not show a greater than 0.6 GI/L change from baseline.

The compound of the invention can be administered in combination with other therapeutic agents other than methotrexate.

In one embodiment, the present invention provides a compound of the invention for use in the treatment of Crohn's disease, wherein the compound is dosed at 200 mg q.d., and wherein prior to treatment, the Crohn's disease patient shows a CDAI score of at least 220 points. In a particular embodiment the patient shows a CDAI score prior to treatment of at least 250 points, at least 300 points, at least 350 points, or at least 400 points. In a particular embodiment, prior to treatment, the Crohn's disease patient shows a CDAI score of at least 250 points, at least 300 points or at least 350 points. In a more particular embodiment, prior to treatment, the Crohn's disease patient shows a CDAI score of at least 250 points, at least 260 points, at least 270 points, at least 280 points, at least 290 points, or at least 300 points.

In one embodiment, the present invention provides a method of treatment of Crohn's disease comprising administering the compound of the invention at a dose of 200 mg q.d., wherein prior to treatment, the Crohn's disease patient shows a CDAI score of at least 220 points. In a particular embodiment the patient shows a CDAI score prior to treatment of at least 250 points, at least 300 points, at least 350 points, or at least 400 points. In a particular embodiment, prior to treatment, the Crohn's disease patient shows a CDAI score of at least 250 points, at least 300 points or at least 350 points. In a more particular embodiment, prior to treatment, the Crohn's disease patient shows a CDAI score of at least 250 points, at least 260 points, at least 270 points, at least 280 points, at least 290 points, or at least 300 points.

In one embodiment, the present invention provides a compound of the invention for use in the treatment of Crohn's disease, wherein the compound is dosed at 200 mg q.d., and wherein a reduction in CDAI score of at least 70 points is seen after 10 weeks of treatment. In a particular embodiment a reduction in the CDAI score of at least 80, at least 90, at least 100, at least 110, at least 120, at least 130, at least 140, at least 150, at least 160, at least 170, at least 180, at least 190, at least 200 or at least 250 points is seen after 10 weeks treatment.

In one embodiment, the present invention provides a compound of the invention for use in the treatment of Crohn's disease, wherein the compound is dosed at 200 mg q.d., and the patient achieves clinical remission (CDAI score <150) after 2 weeks. In a particular embodiment the patient achieves clinical remission after 4 weeks, after 6 weeks, or after 10 weeks. In a most particular embodiment, the compound is dosed at 200 mg q.d., and the patient achieves clinical remission after 10 weeks (CDAI score <150).

In one embodiment, the present invention provides a compound of the invention for use in the treatment of IBD, wherein the patient is a TNF naïve patient. In a particular embodiment, the present invention provides a compound of the invention for use in the prophylaxis and/or treatment of Crohn's disease, wherein the patient is a TNF naïve patient.

In another more particular embodiment, the present invention provides a compound of the invention for use in the prophylaxis and/or treatment of IBD, wherein the patient is a TNF experienced patient. In another more particular embodiment, the present invention provides a compound of the invention for use in the prophylaxis and/or treatment of Crohn's disease, wherein the patient is a TNF experienced patient. In an alternative aspect, the patient is a TNF experienced patient, wherein the anti-TNF therapy is selected from infliximab, adalimumab, golimumab, certolizumab and certolizumab pegol.

In one embodiment, the present invention provides a compound of the invention for use in the treatment of Crohn's disease, wherein the patient has a serum CRP level of 10 mg/L prior to treatment with the compound of the invention.

In another embodiment, the present invention provides a compound of the invention for use in the treatment of Crohn's disease, wherein prior to and/or concomitantly with treatment with the compound of the invention, the patient is receiving treatment using corticosteroids. In particular, the corticosteroid is selected from hydrocortisone, methylprednisolone, prednisone, prednisolone, or budesonide. In a more particular embodiment, the patient is concomitantly receiving and/or has received a daily corticosteroid dose of 20 to 30 mg/day prednisolone/equivalent. In a most particular embodiment, the patient is receiving a daily corticosteroid dose of 20, 21, 22, 23, 24, or 25 mg/day prednisolone/equivalent.

In another aspect, the present invention provides a compound of the invention for use in the treatment of Crohn's disease, wherein prior to and/or concomitantly with treatment with the compound of the invention, the patient is receiving treatment with 5-aminosalicylates. In particular, the 5-aminosalicylate is selected from sulfasalazine and mesalamine.

In one embodiment, the compound of the invention is administered in combination with a therapeutic agent for the treatment of inflammatory conditions selected from: immunoregulatory agents e.g. azathioprine, corticosteroids (e.g. prednisolone or dexamethasone), cyclophosphamide, cyclosporin A, tacrolimus, mycophenolate, mofetil, muromonab-CD3 (OKT3, e.g. Orthocolone®), ATG, aspirin, acetaminophen, ibuprofen, naproxen, and piroxicam.

In one embodiment, the compound of the invention is administered in combination with another therapeutic agent for the treatment and/or prophylaxis of arthritis (e.g. rheumatoid arthritis), particular agents include but are not limited to analgesics, non-steroidal anti-inflammatory drugs (NSAIDS), steroids (e.g. prednisolone), synthetic DMARDS (for example but without limitation methotrexate, leflunomide, sulfasalazine, auranofin, sodium aurothiomalate, penicillamine, chloroquine, hydroxychloroquine, azathioprine, tofacitinib, baricitinib, fostamatinib, and cyclosporin), and biological DMARDS (for example but without limitation infliximab, etanercept, adalimumab, rituximab, and abatacept). Particular agents include steroids (e.g. prednisolone) and NSAIDs.

In one embodiment, a compound of the invention is co-administered with another therapeutic agent for the treatment and/or prophylaxis of inflammatory bowel disease (IBD), particular agents include but are not limited to: glucocorticoids (e.g. prednisone, budesonide) synthetic disease modifying, immunomodulatory agents (e.g. methotrexate, leflunomide, sulfasalazine, mesalazine, azathioprine, 6-mercaptopurine and cyclosporin) and biological disease modifying, immunomodulatory agents (infliximab, adalimumab, rituximab, and abatacept).

By co-administration is included any means of delivering two or more therapeutic agents to the patient as part of the same treatment regime, as will be apparent to the skilled person. Whilst the two or more agents may be administered simultaneously in a single formulation, i.e. as a single pharmaceutical composition, this is not essential. The agents may be administered in different formulations and at different times.

### Clauses

1. A compound according to Formula I:

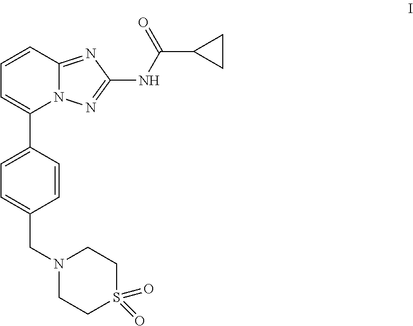 [SMILES: O=C(Nc1nc2cccc(-c3ccc(CN4CCS(=O)(=O)CC4)cc3)n2n1)C1CC1]

or a pharmaceutically acceptable salt thereof, or a solvate or the salt of a solvate thereof, or an active metabolite thereof for use in the treatment of inflammatory conditions.
2. The pharmaceutically acceptable salt of a solvate for use according to clause 1, wherein said salt of a solvate is a [Compound according to Formula I:HCl:3H₂O] adduct.
3. A compound according to Formula II:

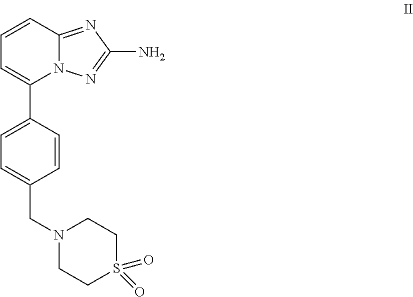 [SMILES: Nc1nc2cccc(-c3ccc(CN4CCS(=O)(=O)CC4)cc3)n2n1]

or a pharmaceutically acceptable salt thereof, or a solvate or the salt of a solvate thereof, for use in the treatment of inflammatory conditions.
4. The compound or pharmaceutically acceptable salt thereof, for use according to clause 1, 2, or 3 wherein the compound is dosed at a dose selected from 25 mg twice per day (b.i.d.), 50 mg once a day (q.d.), 50 mg b.i.d., 100 mg q.d., 100 mg b.i.d., and 200 mg q.d.
5. The compound or pharmaceutically acceptable salt thereof, for use according to clause 1, 2, 3 or 4, wherein the compound is dosed once or twice a day for a period of at least 4 weeks.
6. The compound or pharmaceutically acceptable salt thereof, for use according to clause 5, wherein the compound is dosed once or twice a day for a period of at least 8 weeks.
7. The compound or pharmaceutically acceptable salt thereof, for use according to clause 7, wherein the compound is dosed once or twice a day for a period of at least 12 weeks.
8. The compound or pharmaceutically acceptable salt thereof, for use according to clause 1, 2 or 3 in the treatment of IBD wherein the compound is first administered at an induction dose selected from 100 mg twice per day (b.i.d.), or 200 mg once a day (q.d.) for a period of 4-12 weeks, followed by a maintenance dose selected from 50 mg b.i.d., 100 mg q.d., 100 mg b.i.d., and 200 mg q.d. for a period of at least 4 weeks.
9. The compound or pharmaceutically acceptable salt thereof, for use according to clause 8, wherein the induction dose is administered for 8-12 weeks.
10. The compound or pharmaceutically acceptable salt thereof, for use according to clause 8, wherein the induction dose is administered for 10 weeks.
11. The compound or pharmaceutically acceptable salt thereof, for use according to clause 8, wherein the compound of the invention is administered at an induction dose of 200 mg q.d. for 10 weeks, followed by a maintenance dose of 100 mg q.d. or 200 mg q.d. for at least 4 weeks.
12. The compound, or pharmaceutically acceptable salt thereof for use according to any one of clauses 1 to 11, wherein the inflammatory condition is rheumatoid arthritis.
13. The compound, or pharmaceutically acceptable salt thereof, for use according to any of clauses 1 to 11 wherein the inflammatory condition is inflammatory bowel diseases (IBD) (e.g. Crohn's disease or ulcerative colitis).
14. The compound, or pharmaceutically acceptable salt thereof, for use according to any of clauses 1 to 11 wherein the inflammatory condition is Crohn's disease.
15. The compound, or pharmaceutically acceptable salt thereof for use according to any one of clauses 1 to 14, where the patients have previously had an insufficient response to methotrexate.
16. The compound, or pharmaceutically acceptable salt thereof for use according to any one of clauses 1 to 14, where the patient has previously had an insufficient response to methotrexate.
17. The compound, or pharmaceutically acceptable salt thereof for use according to any one of clauses 1 to 14, in a patient that has previously had an insufficient response to methotrexate.
18. The compound or pharmaceutically acceptable salt thereof for use according to any one of clauses 1 to 14 wherein the patients are concomitantly treated with methotrexate.
19. The compound or pharmaceutically acceptable salt thereof for use according to any one of clauses 1 to 14, or 16 wherein the patient is concomitantly treated with methotrexate.
20. The compound or pharmaceutically acceptable salt thereof for use according to any one of clauses 1 to 14, or 17, in a patient that is concomitantly treated with methotrexate.
21. The compound or pharmaceutically acceptable salt thereof for use according to clause 18, wherein the patients receive between 7.5-25 mg once per week of methotrexate.
22. The compound or pharmaceutically acceptable salt thereof for use according to clause 19 or 20, wherein the patient receives between 7.5-25 mg once per week of methotrexate.
23. The compound or pharmaceutically acceptable salt thereof for use according to clause 18, wherein the patients receive between 10-25 mg once per week of methotrexate.
24. The compound or pharmaceutically acceptable salt thereof for use according to clause 19 or 20, wherein the patient receives between 10-25 mg once per week of methotrexate.
25. The compound or pharmaceutically acceptable thereof for use according to any one of clauses 1 to 15 wherein the patients are not concomitantly treated with methotrexate.
26. The compound or pharmaceutically acceptable thereof for use according to any one of clauses 1 to 14 or 16 wherein the patient is not concomitantly treated with methotrexate.
27. The compound or pharmaceutically acceptable thereof for use according to any one of clauses 1 to 14 or 17, in a patient that is not concomitantly treated with methotrexate.
28. The compound or pharmaceutically acceptable salt thereof for use according to clause 24 wherein the patients do not concomitantly receive any additional treatments for rheumatoid arthritis or IBD (e.g. Crohn's disease or ulcerative colitis).
29. The compound or pharmaceutically acceptable salt thereof for use according to clause 25 or 26, wherein the patient does not concomitantly receive any additional treatments for rheumatoid arthritis or IBD (e.g. Crohn's disease or ulcerative colitis).
30. The compound or pharmaceutically acceptable salt thereof, for use according to any one of clauses 1 to 28, wherein an increase in the levels of haemoglobin are seen after 4 weeks of treatment.
31. The compound or pharmaceutically acceptable salt thereof, for use according to clause 29, wherein an increase of at least 1 g/L is seen.
32. The compound or pharmaceutically acceptable salt thereof, for use according to clause 29, wherein an increase of at least 3.5 g/L is seen in patients administered the compound or a pharmaceutically acceptable salt thereof at a dose of either 100 mg b.i.d. or 200 mg q.d.
33. The compound or pharmaceutically acceptable thereof for use according to any one of clauses 1 to 14, wherein the patient is a TNF naïve patient.
34. The compound or pharmaceutically acceptable thereof for use according to any one of clauses 1 to 14, wherein the patient is a TNF experienced patient.
35. The compound or pharmaceutically acceptable thereof for use according to any one of clauses 1 to 14, wherein the patient is a TNF experienced patient, wherein the anti-TNF therapy is selected from infliximab, adalimumab, golimumab and certolizumab pegol.
36. The compound or pharmaceutically acceptable thereof for use according to any one of clauses 1 to 14, wherein the patient is concomitantly treated with corticosteroids.
37. The compound or pharmaceutically acceptable thereof for use according to any one of clauses 1 to 14, wherein the patient is concomitantly treated with corticosteroids selected from hydrocortisone, methylprednisolone, prednisone, prednisolone, and budesonide.
38. The compound or pharmaceutically acceptable thereof for use according to any one of clauses 1 to 14, wherein the patient is concomitantly receiving and/or has received a daily corticosteroid dose of 20 to 30 mg/day prednisolone/equivalent.

### Pharmaceutical Compositions

When employed as a pharmaceutical, a compound of the invention is typically administered in the form of a pharmaceutical composition. Such compositions can be prepared in a manner well known in the pharmaceutical art and comprise at least one active compound of the invention according to Formula I. Generally, a compound of the invention is administered in a pharmaceutically effective amount. The amount of compound of the invention actually administered will typically be determined by a physician, in the light of the relevant circumstances, including the condition to be treated, the chosen route of administration, the actual compound of the invention administered, the age, weight, and response of the individual patient, the severity of the patient's symptoms, and the like.

The pharmaceutical compositions of this invention can be administered by a variety of routes including oral, rectal, transdermal, subcutaneous, intra-articular, intravenous, intramuscular, and intranasal. Depending on the intended route of delivery, a compound of the invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention according to Formula I is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compound of the inventions of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint or orange flavoring.

Injectable compositions are typically based upon injectable sterile saline or phosphate-buffered saline or other injectable carriers known in the art. As before, the active compound of the invention according to Formula I in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pa., which is incorporated herein by reference.

A compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention according to Formula I may be admixed as a dry powder with a dry gelatin binder in an approximate 1:2 weight ratio. A minor amount of magnesium stearate may be added as a lubricant. The mixture may be formed into 300 mg tablets (100 mg of active compound of the invention according to Formula I per tablet) in a tablet press.

### Formulation 2—Capsules

A compound of the invention according to Formula I may be admixed as a dry powder with a starch diluent in an approximate 1:1 weight ratio. The mixture may be filled into 200 mg capsules (100 mg of active compound of the invention according to Formula I per capsule).

### Formulation 3—Liquid

A compound of the invention according to Formula I (100 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

A compound of the invention according to Formula I may be admixed as a dry powder with a dry gelatin binder in an approximate 1:2 weight ratio. A minor amount of magnesium stearate may be added as a lubricant. The mixture may be formed into 300-600 mg tablets (100-200 mg of active compound of the invention according to Formula I) in a tablet press.

### Formulation 5—Injection

A compound of the invention according to Formula I may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of A compound of the invention according to Formula I (100 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### Chemical Synthetic Procedures

### General

The compound of the invention can be prepared from readily available starting materials using the following general methods and procedures. It will be appreciated that where typical or preferred process conditions (i.e. reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art (Greene, T W; Wuts, P G M;, 1991).

The following methods are presented with details as to the preparation of a compound of the invention as defined hereinabove and the comparative examples. A compound of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm). ¹H NMR spectra were recorded on a Bruker DPX 400 NMR spectrometer (400 MHz or a Bruker Advance 300 NMR spectrometer (300 MHz). Chemical shifts (δ) for 1H NMR spectra are reported in parts per million (ppm) relative to tetramethylsilane (δ 0.00) or the appropriate residual solvent peak, i.e. CHCl₃ (δ 7.27), as internal reference. Multiplicities are given as singlet (s), doublet (d), triplet (t), quartet (q), quintuplet (quin), multiplet (m) and broad (br). Electrospray MS spectra were obtained on a Waters platform LC/MS spectrometer or with Waters Acquity H-Class UPLC coupled to a Waters Mass detector 3100 spectrometer. Columns used: Waters Acquity UPLC BEH C18 1.7 ηm, 2.1 mm ID×50 mm L, Waters Acquity UPLC BEH C18 1.7 μm, 2.1 mm ID×30 mm L, or Waters Xterra MS 5 μm C18, 100×4.6 mm. The methods are using either MeCN/H₂O gradients (H₂O contains either 0.1% TFA or 0.1% NH₃) or MeOH /H₂O gradients (H₂O contains 0.05% TFA). Microwave heating was performed with a Biotage Initiator.

TABLE I
List of abbreviations used in the experimental section:

| Abbreviation | Definition |
|---|---|
| μL | microliter |
| μM | micromolar |
| br s | broad singlet |
| DCM | Dichloromethane |
| DMF | N,N-dimethylformamide |
| DMSO | Dimethylsulfoxide |
| cpm | Counts per minute |
| Et₂O | Diethyl ether |
| EtOAc | Ethyl acetate |
| FBS | Fetal bovine serum |
| eq | equivalents |
| g | gram |
| h | hour |
| LCMS | Liquid Chromatography- Mass Spectrometry |
| m | multiplet |
| MeCN | Acetonitrile |
| mg | milligram |
| min | minute |
| mL | milliliter |
| MHz | megahertz |
| N | Normal |
| NMR | Nuclear Magnetic Resonnance |
| PdCl₂dppf | [1,1′-Bis(diphenylphosphino)ferrocene] dichloro- |
| | palladium(II) |
| ppm | part-per-million |
| q | quadruplet |
| RNA | Ribonucleic acid |
| q.d. | quo die (once a day) |
| s | singlet |
| b.i.d. | bis in die (twice daily) |
| shRNA | short hairpin RNA |
| t | triplet |
| TFA | Trifluoroacetic acid |
| THF | Tetrahydrofuran |
| v:v | volume:volume |
| CRP | c-Reactive Protein |
| NRI | nonresponder imputation |
| LOCF | last-observation-carried-forward |

### SYNTHETIC PREPARATION OF THE COMPOUND OF THE INVENTION

### Example 1

### Preparation of Compound 1

### 1.1. Route 1

### 1.1.1. 4-[4-(4,4,5,5-Tetramethyl-[1,3,2]dioxaborolan-2-yl)-benzyl]-thiomorpholine-1,1-dioxide

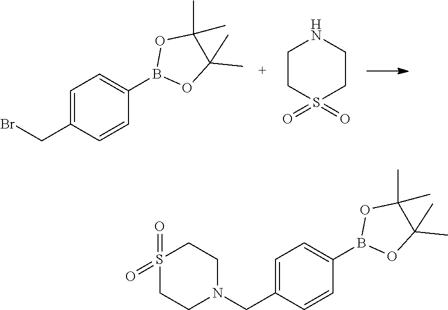 [SMILES: CC1(C)OB(c2ccc(CBr)cc2)OC1(C)C ; O=S1(=O)CCNCC1 ; CC1(C)OB(c2ccc(CN3CCS(=O)(=O)CC3)cc2)OC1(C)C]

2-(4-Bromomethyl-phenyl)-4,4,5,5-tetramethyl-[1,3,2]dioxaborolane (1 eq) and DIPEA (2 eq) are dissolved in DCM/MeOH (5:1 v:v) under N₂ and thiomorpholine 1,1-dioxide (2 eq) is added portionwise. The resulting solution is stirred at room temperature for 16 h. After this time, the reaction is complete. The solvent is evaporated. The compound is extracted with EtOAc and water, washed with brine and dried over anhydrous MgSO₄. Organic layers are filtered and evaporated. The final compound is isolated without further purification.

### 1.1.2. Cyclopropanecarboxylic acid (5-bromo-[1,2,4]triazolo[1,5-a]pyridin-2-yl)-amide

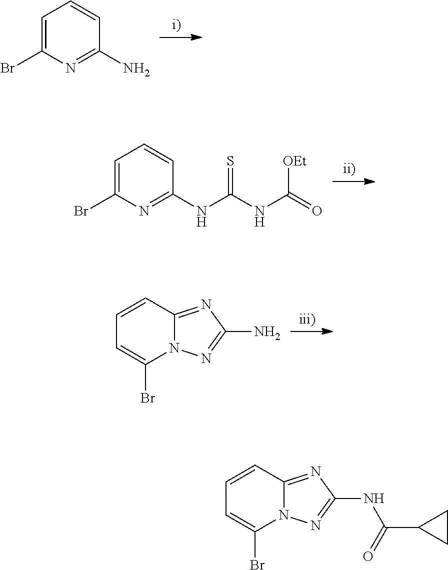 [SMILES: Nc1cccc(Br)n1 ; CCOC(=O)NC(=S)Nc1cccc(Br)n1 ; Nc1nc2cccc(Br)n2n1 ; O=C(Nc1nc2cccc(Br)n2n1)C1CC1]

### 1.1.2.1. Step i): 1-(6-Bromo-pyridin-2-yl)-3-carboethoxy-thiourea

To a solution of 2-amino-6-bromopyridine (1) (253.8 g, 1.467 mol) in DCM (2.5 L) cooled to 5° C. is added ethoxycarbonyl isothiocyanate (173.0 mL, 1.467 mol) dropwise over 15 min. The reaction mixture is then allowed to warm to room temp. (20° C.) and stirred for 16 h. Evaporation in vacuo gives a solid which may be collected by filtration, thoroughly washed with petrol (3×600 mL) and air-dried to afford the desired product. The thiourea may be used as such for the next step without any purification.

¹H (400 MHz, CDCl₃) δ 12.03 (1H, br s), 8.81 (1H, d), 8.15 (1H, br s), 7.60 (1H, t), 7.32 (1H, dd), 4.31 (2H, q), 1.35 (3H, t).

### 1.1.2.2. Step ii): 5-Bromo-[1,2,4]triazolo[1,5-a]pyridin-2-ylamine

To a suspension of hydroxylamine hydrochloride (101.8 g, 1.465 mol) in EtOH/MeOH (1:1, 900 mL) is added N,N-diisopropylethylamine (145.3 mL, 0.879 mol) and the mixture is stirred at room temp. (20° C.) for 1 h. 1-(6-Bromo-pyridin-2-yl)-3-carboethoxy-thiourea (2) (89.0 g, 0.293 mol) is then added and the mixture slowly heated to reflux (Note: bleach scrubber is required to quench H₂S evolved). After 3 h at reflux, the mixture is allowed to cool and filtered to collect the precipitated solid. Further product is collected by evaporation in vacuo of the filtrate, addition of H₂O (250 mL) and filtration. The combined solids are washed successively with H₂O (250 mL), EtOH/MeOH (1:1, 250 mL) and Et₂O (250 mL) then dried in vacuo to afford the triazolopyridine derivative (3) as a solid. The compound may be used as such for the next step without any purification.

¹H (400 MHz, DMSO-d₆) δ 7.43-7.34 (2H, m, 2 x aromatic-H), 7.24 (1H, dd, J 6.8 and 1.8 Hz, aromatic-H), 6.30 (2H, br, NH2); m/z 213/215 (1:1, M+H+, 100%).

### 1.1.2.3. Step iii): Cyclopropanecarboxylic acid (5-bromo-[1,2,4]triazolo[1,5-]pyridin-2-yl)-amide

To a solution of the 2-amino-triazolopyridine obtained in the previous step (7.10 g, 33.3 mmol) in dry MeCN (150 mL) at 5° C. is added Et₃N (11.6 mL, 83.3 mmol) followed by cyclopropanecarbonyl chloride (83.3 mmol). The reaction mixture is then allowed to warm to ambient temperature and stirred until all starting material is consumed. If required, further Et₃N (4.64 mL, 33.3 mmol) and cyclopropanecarbonyl chloride (33.3 mmol) is added to ensure complete reaction. Following solvent evaporation in vacuo the resultant residue is treated with 7 N methanolic ammonia solution (50 mL) and stirred at ambient temp. (for 1 h-16 h) to hydrolyse any bis-acylated product. Product isolation is made by removal of volatiles in vacuo followed by trituration with Et₂O (50 mL). The solids are collected by filtration, washed with H₂O (2×50 mL), acetone (50 mL) and Et₂O (50 mL), then dried in vacuo to give the desired compound.

### 1.1.3. Compound 1

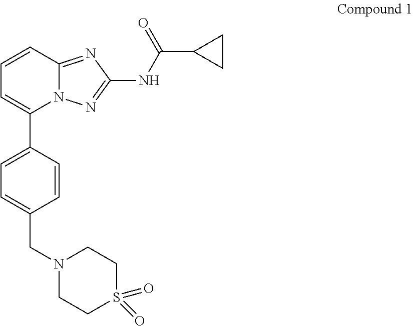 [SMILES: O=C(Nc1nc2cccc(-c3ccc(CN4CCS(=O)(=O)CC4)cc3)n2n1)C1CC1]

4-[4-(4,4,5,5-Tetramethyl- [1,3,2]dioxaborolan-2-yl)-benzyl]-thiomorpholine-1,1-dioxide (1.1 eq.) is added to a solution of cyclopropanecarboxylic acid (5-bromo-[1,2,4]triazolo[1,5-a]pyridin-2-yl)-amide in 1,4-dioxane/water (4:1). K₂CO₃ (2 eq.) and PdCl₂dppf (0.03 eq.) are added to the solution. The resulting mixture is then heated in an oil bath at 90° C. for 16 h under N₂. Water is added and the solution is extracted with ethyl acetate. The organic layers are dried over anhydrous MgSO₄ and evaporated in vacuo. The final compound is obtained after purification by flash chromatography.

Alternatively, after completion of the reaction, a palladium scavenger such as 1,2-bis(diphenylphosphino)ethane, is added, the reaction mixture is allowed to cool down and a filtration is performed. The filter cake is reslurried in a suitable solvent (e.g. acetone), the solid is separated by filtration, washed with more acetone, and dried. The resulting solid is resuspended in water, aqueous HCl is added, and after stirring at room temperature, the resulting solution is filtered on celite (Celpure P300). Aqueous NaOH is then added to the filtrate, and the resulting suspension is stirred at room temperature, the solid is separated by filtration, washed with water and dried by suction. Finally the cake is re-solubilised in a mixture of THF/H₂O, treated with a palladium scavenger (e.g. SMOPEX 234) at 50° C., the suspension is filtered, the organic solvents are removed by evaporation, and the resulting slurry is washed with water and methanol, dried and sieved, to obtain the desired compound as a free base.

### 1.2. Route 2

### 1.2.1. Step 1: cyclopropanecarboxylic acid [5-(4-hydroxymethyl-phenyl)-[1,2,4]triazolo[1,5-]pyridin-2-yl]-amide

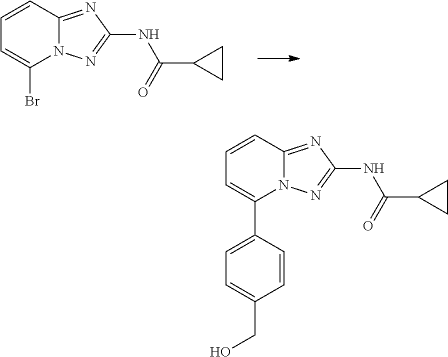 [SMILES: O=C(Nc1nc2cccc(Br)n2n1)C1CC1 ; O=C(Nc1nc2cccc(-c3ccc(CO)cc3)n2n1)C1CC1]

4-(Hydroxymethyl)phenylboronic acid (1.1 eq.) is added to a solution of cyclopropanecarboxylic acid (5-bromo-[1,2,4]triazolo[1,5-a]pyridin-2-yl)-amide in 1,4-dioxane/water (4:1). K₂CO₃ (2 eq.) and PdCl₂dppf (0.03 eq.) are added to the solution. The resulting mixture is then heated in an oil bath at 90° C. for 16 h under N₂. Water is added and the solution is extracted with ethyl acetate. The organic layers are dried over anhydrous MgSO₄ and evaporated in vacuo. The resulting mixture is used without further purification.

### 1.2.2. Step 2: Cyclopropanecarboxylic acid [5-(4-bromomethyl-phenyl)-[1,2,4]triazolo[1,5-]pyridin-2-yl]-amide

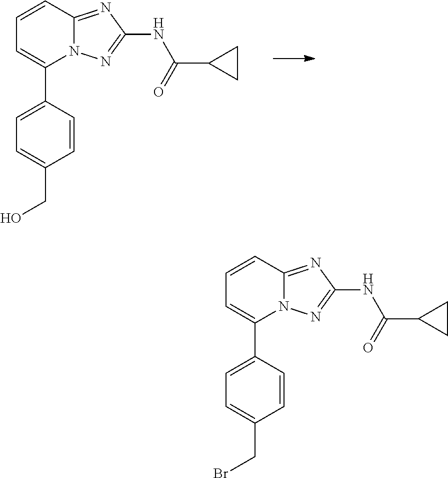 [SMILES: O=C(Nc1nc2cccc(-c3ccc(CO)cc3)n2n1)C1CC1 ; O=C(Nc1nc2cccc(-c3ccc(CBr)cc3)n2n1)C1CC1]

To a solution of cyclopropanecarboxylic acid [5-(4-hydroxymethyl-phenyl)-[1,2,4]triazolo[1,5-a]pyridin-2-yl]-amide (1.0 eq) in chloroform is slowly added phosphorus tribromide (1.0 eq.). The reaction mixture is stirred at room temperature for 20 h, quenched with ice and water (20 mL) and extracted with dichloromethane. The organic layer is dried over anhydrous MgSO₄, filtered and concentrated to dryness. The resulting white residue is triturated in dichloromethane/diethyl ether 2:1 to afford the desired product.

### 1.2.3. Step 3

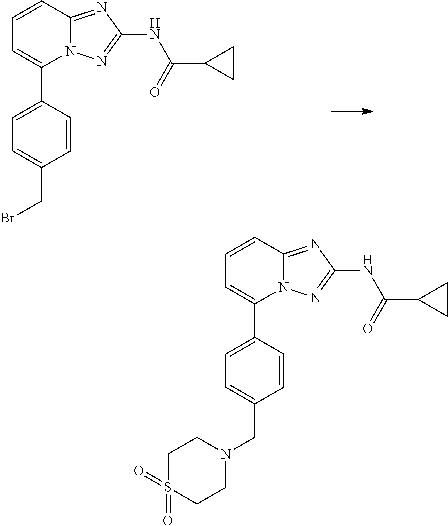 [SMILES: O=C(Nc1nc2cccc(-c3ccc(CBr)cc3)n2n1)C1CC1 ; O=C(Nc1nc2cccc(-c3ccc(CN4CCS(=O)(=O)CC4)cc3)n2n1)C1CC1]

Cyclopropanecarboxylic acid [5-(4-bromomethyl-phenyl)- [1,2,4]triazolo [1,5-a]pyridin-2-yl]-amide (1 eq) and DIPEA (2 eq) are dissolved in DCM/MeOH (5:1 v:v) under N2 and thiomorpholine 1,1-dioxide (1.1 eq) is added dropwise. The resulting solution is stirred at room temperature for 16 h. After this time, the reaction is complete. The solvent is evaporated. The compound is dissolved in DCM, washed with water and dried over anhydrous MgSO₄. Organic layers are filtered and evaporated. The final compound is isolated by column chromatography using EtOAc to afford the desired product.

### 1.3. Preparation of the [compound 1:HCl:3H₂O] adduct

The identification and preparation of the salt and solvates of Compound 1 are disclosed in PCT application PCT/EP2015/052242 filed on the 4 Feb. 2015.

### 1.3.1. Protocol 1

To Compound 1 (44 kg, 1.0 eq) under inert atmosphere, is added water (15 rel vol, 1000 L), and the mixture is stirred at 50° C. 3.5 eq. aq HCl (5 rel vol) is added over 10-15 min, at a maximum temperature of 55° C. Upon completion of the addition, the stirring is continued at 50° C. for 15 min, and the reaction is then cooled to 15° C. and stirred at that temperature for at least 12 h but no more than 24 h.

The resulting solid is separated by filtration, and the cake is washed with water (2.0 rel vol)., and the cake is dried under nitrogen for at least 4 h to afford the desired product.

### 1.3.2. Protocol 2

To Compound 1 (45g, 106 mmol, 1 eq.) under inert atmosphere is added DCM (675 mL) and methanol (225 mL). The resulting suspension is heated to 35° C. under stirring, and trimercaptotriazine trisodium salt 15% in water (22.5 g, 14 mmol, 0.13 eq) is added, and the resulting solution is stirred for 5 h, after which the solution is filtered on 0.45 μm paper under nitrogen pressure.

To the filtrate is added water (50 mL), and the resulting biphasic mixture is stirred at 35° C. for 15 min, after which period the phases are separated, and the organic layer is allowed to cool down to 20° C., and washed twice more with 50 mL water.

The organic layer is cooled down to 15-20° C., then HCl 10% in methanol (42.4 g, 116 mmol, 1.10 eq.) is added over 30 min, causing the precipitation of a solid. The suspension is further stirred at 20° C. for 3 h, then the precipitate is isolated by filtration, the cake is washed with methanol (2×50 mL) to afford the desired compound, which is dried under vacuum at 45° C. for 3 h. The cake is then resuspended in water (220 mL) and stirred for 6 h at 50° C., and then cooled to 15-20° C. The resulting solid is separated by filtration and the cake is washed with water (2×30 mL), and dried at 45° C. for 3 h to afford the desired product.

### 1.3.3. Protocol 3

### 1.3.3.1. Step 1: Compound 1.HaMeOH

To Compound 1 (100 g, 235 mmol, 1 eq.) suspended in DCM (1.5 L), is added MeOH (0.5 L), and the resulting solution is heated to 35° C. Trimercaptotriazine trisodium 85% (8.7 g, 3 mmol, 0.13 eq.) in water (42 mL) is added and the resulting mixture is stirred at 35° C. for at least 5 h. The solution is then filtered on a 0.45 μm paper filter under nitrogen pressure.

To the resulting solution is added water (150 g), stirred at 35° C. for 15 to 30 min, and the biphasic mixture is separated. The organic layer is washed again twice with water (2×150 g).

Finally, a solution of HCl in MeOH (10% w/w) (141 g) is added, and the suspension is stirred at 20° C. for 3 h, and the resulting solid is separated by filtration, the cake is washed with MeOH (2×118 g), dried under vacuum for 3 h at 45° C., to afford Compound 1.HCl.MeOH.

### 1.3.3.2. Step 2: Compound 1.HCl.3H₂O

To formic acid (200 g, 1.6 eq) in water (36 g, 0.4 eq.) is added Compound 1.HCl.MeOH (100 g, 1 eq.) obtained in Step 1 above. The resulting mixture is heated to 55° C. under stirring, and the solution is filtered through a 0.45 μm filter cartridge. Formic acid 85% aq (200 g) is added, and the mixture is cooled to 28-32° C. under gentle stirring.

Water (100 g) is then added, followed with Compound 1.HCl.3H₂O (1 g) causing the precipitation of Compound 1.HCl.1.5HCO₂H.

Under stirring at 28-32° C., water (2L) is added portionwise in 8 portions of 100 mL, 1 portion of 200 mL, and 2 portions of 500 mL.

The resulting suspension is then filtered, the cake is washed with water (2×100 mL) and dried at 30-35° C. to yield Compound 1.HCl.3H₂O.

### BIOLOGICAL EXAMPLES

The compound of the invention according to Formula I has been extensively profiled, and data are disclosed in (Menet and Smits, 2010). The synthesis of the salt and suitable formulations have been described in PCT/EP2015/052239, and in PCT/EP2015/052242.

Similarly, the compound of the invention according to Formula I has been extensively profiled, and data are disclosed in WO 2013/189771 (Van t 'Klooster et al., 2013).

### Example 2

### Clinical Setting

### 2.1. Study 1—RA Patients with Inadequate Response to Methotrexate

### 2.1.1. Study Design

Double-blind, placebo-controlled add on study in subjects with moderately to severely active RA who have an inadequate response to methotrexate (MTX) (oral or parenteral).

595 subjects randomized to one of 6 dose regimens of Compound 1 (dosed as [Compound 1:HCl:3H₂O]) (3 dose levels administered either once or twice daily) or placebo on top of each subject's stable dose of MTX.

### 2.1.2. Study Duration

Treatment duration: 24 weeks.

### 2.1.3. Treatment

Compound 1 (dosed as [Compound 1:HCl:3H₂O]) is dosed for twelve weeks once daily (q.d.) (50 mg, 100 mg or 200 mg) or twice daily (b.i.d.) (25 mg, 50 mg or 100 mg); or placebo.

At Week 12, the subjects on placebo who have not achieved 20% improvement in swollen joint count (SJC66) and tender joint count (TJC68) are re-randomized automatically to receive Compound 1 (dosed as a [Compound 1:HCl:3H₂O]) either at 100 mg q.d. or 50 mg b.i.d. doses in a blinded fashion; subjects on 50 mg q.d. who have not achieved 20% improvement in SJC66 and TJC68 will be assigned to 100 mg q.d. and subjects on 25 mg b.i.d. that have not achieved a 20% improvement in SJC66 and TJC68 will be assigned to 50 mg b.i.d. Subjects who switch treatment at week 12 are handled as if they discontinued at week 12 for the purpose of statistical analysis, whereas subjects in the other groups will maintain their randomized treatment until Week 24.

### 2.1.4. Participants

### 2.1.4.1. Main Inclusion Criteria

male or female subjects who are ≥18 years of age, on the day of signing informed consent,
diagnosis of RA at least 6 months prior to screening and meeting the 2010 ACR/EULAR criteria of RA and ACR functional class I-III (Aletaha et al., 2010),
≥6 swollen joints (from a 66 joint count) and ≥8 tender joints (from a 68 joint count) at Screening and at Baseline,
screening serum c-reactive protein (CRP) ≥0.7×upper limit of laboratory normal range (ULN),
on MTX for ≥6 months and on a stable dose (15 to 25 mg/week) of MTX for at least 4 weeks prior to Screening and continued on their current regimen for the duration of the study. Stable doses of MTX as low as 10 mg/week are allowed when there is documented evidence of intolerance or safety issues at higher doses.

### 2.1.4.2. Main Exclusion Criteria

current therapy with any disease-modifying anti-rheumatic drugs (DMARD) other than MTX, including oral or injectable gold, sulfasalazine, antimalarials, azathioprine, or D-penicillamine within 4 weeks prior to Baseline, cyclosporine within 8 weeks prior to Baseline, and leflunomide within 3 months prior to Baseline or a minimum 4 weeks prior to Baseline if after 11 days of standard cholestyramine therapy,
current or previous RA treatment with a biologic DMARD, with the exception of biologic DMARDs administered in a single clinical study setting more than 6 months prior to Screening (12 months for rituximab or other B cell depleting agents), where the biologic DMARD was effective, and if discontinued, this should not be due to lack of efficacy,
previous treatment at any time with a cytotoxic agent, other than MTX, before Screening.

### 2.2. Study 2—RA Patients Monotherapy with Compound 1

### 2.2.1. Purpose of the Study

Randomized, double-blind, placebo-controlled, multicenter, phase IIb dose finding study of Compound 1 (dosed as a [Compound 1:HCl:3H₂O]) administered for 24 weeks as monotherapy to subjects with moderately to severely active rheumatoid arthritis who have an inadequate response to methotrexate alone

### 2.2.2. Study Design

Double-blind, placebo-controlled, monotherapy study in subjects with moderately to severely active RA who have an inadequate response to methotrexate (MTX) (oral or parenteral).

280 subjects randomized to one of 3 doses of Compound 1 (dosed as a [Compound 1:HCl:3H₂O]) or to placebo, given once daily (q.d.).

### 2.2.3. Study Duration

Treatment duration: 24 weeks.

### 2.2.4. Treatment

Twelve weeks of treatment with Compound 1 (dosed as a [Compound 1:HCl:3H₂O]) at 50 mg, 100 mg, or 200 mg q.d.; or placebo. At Week 12, all subjects on placebo and the subjects on the 50 mg dose who have not achieved 20% improvement in swollen joint count (SJC66) and tender joint count (TJC68) will be assigned to 100 mg q.d. in a blinded fashion and will continue treatment until Week 24. Subjects in the other groups will maintain their randomized treatment until Week 24.

### 2.2.5. Participants

### 2.2.5.1. Main Inclusion Criteria

The following criteria were used to select patients for inclusion in the trial:
male or female subjects who are ≥18 years of age on the day of signing informed consent,
diagnosis of RA since at least 6 months prior to Screening and meeting the 2010 ACR/EULAR criteria of RA and ACR functional class I-III,
≥6 swollen joints (from a 66-joint count) and ≥8 tender joints (from a 68-joint count) at Screening and at Baseline,
Screening serum c-reactive protein (CRP) ≥0.7×upper limit of laboratory (reference) normal range (ULN),
inadequate response in terms of either lack of efficacy or toxicity to MTX,
washed out from MTX for a period of at least 4 weeks before or during the Screening period.

### 2.2.5.2. Main Exclusion Criteria

The following criteria were used to select patients for exclusion from the trial:
modifying anti-rheumatic drug (DMARD), including oral or injectable gold, sulfasalazine, azathioprine, or D penicillamine within 4 weeks prior to Baseline, cyclosporine within 8 weeks prior to Baseline, and leflunomide within 3 months prior to Baseline or a minimum 4 weeks prior to Baseline if after 11 calendar days of standard cholestyramine therapy, with the exception of antimalarials, which must be at a stable dose for at least 12 weeks prior to Baseline,
current or previous RA treatment with a biologic DMARD, with the exception of biologic DMARDs:administered in a single clinical study setting, and; more than 6 months prior to Screening (12 months for rituximab or other B cell depleting agents), and; where the biologic DMARD was effective, and if discontinued, this should not be due to lack of efficacy,
previous treatment at any time with a cytotoxic agent, other than MTX, before Screening.

### 2.3. Study 3: Crohn's Disease Study with Monotherapy of Compound 1

### 2.3.1. Purpose of the Study

Double-Blind, Randomized, Placebo-Controlled, Multi-Centre Study to Investigate the Efficacy and Safety of Compound 1 in Subjects With Active Crohn's Disease With Evidence of Mucosal Ulceration.

### 2.3.2. Study Design

This is a double-blind, randomized, placebo-controlled, multi-centre Phase 2 study to investigate the efficacy and safety of Compound 1 administered once daily for the treatment of active CD with evidence of mucosal ulceration. The pharmacokinetics (including a PK substudy) and pharmacodynamics of Compound 1 and metabolite in CD will also be characterised.

A total of 180 eligible subjects will be randomized to receive Compound 1 or placebo in addition to their stable background treatment (eg, corticosteroids, aminosalicylates, or CD-related antibiotics). The study will consist of 2 parts, with total treatment duration of 20 weeks. Randomization in Part 1 will be stratified according the subject's previous anti-TNF exposure/response, CRP level at Screening, and oral corticosteroid use at visit Day −1.

After the first 10 weeks of treatment in Part 1, patients will be re-randomized for Part 2 as shown in the diagram below and will be stratified according to the subject's clinical response, previous anti-TNF exposure/response, and oral corticosteroid use at visit Day -1.

### 2.3.3. Study Duration

Maximum of 27 weeks: up to 28 days for Screening, up to 20 weeks for treatment, and 2 weeks for follow-up (+5 days visit window, if applicable).

### 2.3.4. Treatment

A diagram of the study design is shown in Table II below:

TABLE II
Study design

| Week 1-10 | Re-randomization | Week 10-20 |
|---|---|---|
| Placebo (1)* | Responder** | Placebo |
| | Non responder | 100 mg q.d. Compound 1 |
| 200 mg q.d. of | Responder** | 200 mg q.d. Compound 1 (2)* |
| Compound 1 (3)* | | 100 mg q.d. Compound 1 (2)* |
| | | Placebo (1)* |
| | Non responder | 200 mg q.d. Compound 1 (3)* |
| | | Placebo (1)* |

*Randomisation Ratio
**Reduction in CDAI of 100 points

### 2.3.5. Participants

### 2.3.5.1. Main Inclusion Criteria

Subjects should have all of the following conditions at to be eligible for admission into the study:
1. Male or female subjects between the ages of 18 and 75 years, on the day of signing informed consent.
2. Documented history of ileal, colonic, or ileocolonic CD (at least 3 months prior Screening) as assessed by colonoscopy, and supported by histological assessment.
3. Crohn's Disease Activity Index (CDAI) score during Screening ≥220 to ≤450.
4. Evidence of active inflammation at Screening as demonstrated by endoscopic confirmation of active disease (based on central reading) with evidence of ulceration corresponding to a score of 1 in at least 1 of the 5 ileocolonic segments on the Presence of Ulcers subscore of the Simplified Endoscopy Score for CD (SES CD) and total score of at least 7.
5. Treatment with oral steroids (≤30 mg prednisolone equivalent/day or budesonide dose ≤9 mg/day) is allowed, if at a stable dose since at least 2 weeks prior to the first dose of study drug.
6. Subjects previously not exposed to anti-TNF treatment (eg, TNF-naïve) or subjects previously exposed to anti-TNF therapy (infliximab, adalimumab or certolizumab pegol) at a dose registered for the treatment of CD that has been discontinued at least 8 weeks prior to Baseline. Subjects deemed by the treating physician as a primary or secondary non-responder or intolerant to anti-TNF treatment or responders to anti-TNF treatment, where treatment was stopped for other reasons (TNF-experienced) can also be included.
7. Subjects are allowed to continue on concurrent treatment with the following agents:
   a. Mesalazine and olsalazine if stable dosage for at least 4 weeks prior to Screening (same dosage to be maintained throughout the study). Previous exposure to sulfasalazine is permitted but must be discontinued at least 4 weeks prior to Screening in male subjects.
   b. Crohn's Disease-related antibiotics if stable dosage for at least 4 weeks prior to Screening and no discontinuation in the 14 days prior to the first dose of study drug.
   c. Probiotics if stable dosage for 2 weeks prior to the first dose of study drug.
8. Previous exposure to immunomodulators (e.g. thiopurines and methotrexate) is permitted, but must be discontinued (and agreed by the subject) at least 25 days prior to the first dose of the study drug. Subjects whose immunomodulators (e.g. thiopurines and methotrexate) were discontinued prior to Screening are also permitted to participate. In these cases documented evidence for the reasons of discontinuation should be provided.
9. The results of the following laboratory tests during Screening must be as specified below:
   a) Haemoglobin ≥9 g/dL (International System of Units [SI]: ≥90 g/L)
   b) White blood cells (WBCs) ≥3.0×109 cells/L
   c) Neutrophils ≥2.0×109 cells/L
   d) Lymphocytes ≥0.5×109 cells/L
   e) Platelets ≥100×109 cells/L
   f) Serum alanine transaminase (ALT) and aspartate transaminase (AST) ≤1.5×ULN
   g) Total bilirubin level ≤1.5×ULN
   h) Alkaline phosphatase ≤1.5×ULN
   i) Lipase ≤1.5×ULN and amylase ≤1.5×ULN
   j) Creatinine clearance >60 mL/min. Creatinine clearance will be calculated using the Cockroft-Gault formula.
10. Women of childbearing potential must have a negative blood pregnancy test, unless they are surgically sterile, had a hysterectomy, or have been postmenopausal for at least 1 year (12 consecutive months without menses); in case of doubt a determination of serum follicle-stimulating hormone (FSH) can be done with FSH levels >35 mIU/mL confirming menopause status.
11. Subjects willing to use highly effective contraceptive methods prior to the first dose of the study drug, during the study and for at least 12 weeks after the last dose of the study drug.
   a) If the subject is a sexually active woman of childbearing potential, she and her male partner are required to simultaneously use two effective contraceptive methods as listed in section 10.4.8.1.2 of the protocol. Female subjects who wish to use non-hormonal contraception must have done so for at least 14 days prior to the first dose of the study drug.
   b) Non-vasectomized males with female partners of childbearing potential must be willing to use a condom in addition to having their female partner using another form of contraception as listed in section 10.4.8.1.3 of the protocol.
12. Able and willing to give voluntary written informed consent and meet all of the inclusion and none of the exclusion criteria before being enrolled in the study. The subjects must sign the informed consent form prior to any study related procedures and agree to the schedule of assessments (including 2 colonoscopies).
13. Judged to be in good health, except for their CD, as determined by the Investigator based upon the results of medical history, laboratory profile, physical examination, chest X-ray, and a 12-lead electrocardiogram (ECG) performed during Screening.

### 2.3.5.2. Main Exclusion Criteria

Subjects who exhibit any of the following conditions at screening will not be eligible for admission into the study:
1. Diagnosis of indeterminate colitis, ulcerative colitis (UC), or clinical findings suggestive of UC.
2. Stoma, gastric or ileanal pouch, proctocolectomy or total colectomy, symptomatic stenosis or obstructive strictures, abscess or suspected abscess, history of bowel perforation.
3. Subject who has had surgical bowel resections within the past 6 months or is planning any resection at any time point while enrolled in the study.
4. Subject who has short bowel syndrome.
5. Subject who is receiving tube feeding, defined formula diets, or total parenteral alimentation.
6. Subjects with positive Clostridium difficile (C. difficile) toxin stool assay or test positive for stool culture of enteric pathogens, ova or parasites during the screening period.
7. Subject has received nonsteroidal anti-inflammatory drugs (NSAIDs) within 14 days prior to Screening or during screening period.
8. Subject has received therapeutic enema or suppository, other than required for colonoscopy, within 7 days prior to Screening or during screening period.
9. Subject has received intravenous corticosteroids within 14 days prior to Screening or during screening period.
10. If nonsystemic steroids are being used for other conditions than CD, subjects may be included at the discretion of the Investigator after discussion with the Medical Monitor.
11. Treatment with cyclosporine, mycophenolate mofetil, tacrolimus, or interferon within 10 weeks prior to Screening or during screening period.
12. Any prior treatment with lymphocyte-depleting agents (such as CamPath® [Alemtuzumab]). Also subjects who have previously received either lymphocyte apheresis or selective monocyte granulocyte apheresis (e.g. Cellsobra®) within 12 months prior to Screening or during screening period.
13. Subjects who have previously received fecal microbiota transplants or stem cell transplantation.
14. Subjects who have received previous treatment with investigational chemical agents within 4 weeks prior to Screening or during screening period.
15. Subjects who have previously received treatment with biological investigational medicinal products including murine, chimeric or humanized monoclonal antibodies or a chemokine receptor blocker within less than 5 half-lives prior to Baseline. Previous treatment with a janus kinase inhibitor is prohibited.
16. Known hypersensitivity to study drug ingredients or a significant allergic reaction to any drug as determined by the Investigator, such as anaphylaxis requiring hospitalization.
17. Subject with a previous history of dysplasia of the gastrointestinal tract (high or low grade, flat or raised including discrete adenoma-like dysplasia or indefinite dysplasia) or found to have above described dysplasia in any biopsy performed during the Screening colonoscopy.
18. Concurrent gastro-intestinal (GI) malignancy or a history of cancer elsewhere (other than basal cell carcinoma or carcinoma in situ of the cervix successfully treated more than 5 years prior to the initial study drug administration).
19. History of lymphoproliferative disease; or signs and symptoms suggestive of possible lymphoproliferative disease including lymphadenopathy or splenomegaly.
20. Positive serology for human immunodeficiency virus (HIV) 1 or 2 or hepatitis B or C, or any history of HIV or hepatitis from any cause with the exception of hepatitis A.
21. Known active infection of any kind (excluding fungal infections of nail beds), or any major episode of infection requiring hospitalization or treatment with parenteral (intramuscular or IV) anti-infectives (antibiotics, antiviral, anti-fungals or anti-parasitic agents) within 4 weeks of the Screening visit or completion of oral anti-infectives within 2 weeks of the Screening visit (except Crohn's disease-related antibiotics) Immunocompromised subjects who in the opinion of the investigator are at an unacceptable risk for participating in the study.
22. Previous history of symptomatic herpes zoster or herpes simplex infection within 12 weeks prior to Screening or have a history of disseminated/complicated herpes zoster infection (multi-dermatomal involvement, ophthalmic zoster CNS involvement, or postherpetic neuralgia).
23. History of invasive infection (e.g., listeriosis, histoplasmosis).
24. Significant blood loss (>500 mL) or transfusion of any blood product within 4 weeks prior to Screening.
25. Currently on any therapy for chronic infection (such as pneumocystis, CMV, herpes simplex, herpes zoster, or atypical mycobacteria).
26. History of active or latent tuberculosis (TB) infection as determined by:
   a. positive diagnostic TB test result (defined as a positive QuantiFERON TB Gold test) OR
   b. a chest X-ray radiograph (both posterior-anterior and lateral views), taken within 3 months prior to Screening or at Screening and read by a qualified radiologist, with evidence of current active TB or old inactive TB.
27. Administration of a live vaccine within 90 days or an attenuated vaccine within 30 days prior to the initial study drug administration.
28. History within the previous year or current evidence of drug or alcohol abuse according to the opinion of the investigator.
29. Currently pregnant or breastfeeding or not willing to maintain birth control methods for at least 12 weeks after last study drug administration.
30. Medical, psychiatric, cognitive, or other conditions that, according to the Investigator's medical judgment, compromise the subject's ability to understand the subject information, to give informed consent, to comply with the requirements of the study protocol (that is likely to affect the subject's return for visits on schedule), or ability to complete the study.
31. If applicable to national or local legislation: history of being admitted to an institution under an administrative or court order.
32. Any concurrent illness, disability, or clinically significant abnormality (including laboratory tests) that may affect the interpretation of clinical safety or efficacy data or prevent the subject from safely completing the assessments required by the protocol as judged by the Investigator.

### Example 3

### In Vivo Assays

Aspartate Aminotransferase (AST) is found in the liver, heart, skeletal muscle, kidneys, brain, and red blood cells, whereas Alanine Aminotransferase (ALT) is found in plasma and in various body tissues, but is most common in the liver. Serum AST and ALT levels, and their ratio (AST/ALT ratio) are commonly measured clinically as biomarkers for liver health.

### 3.1. Aspartate Aminotransferase (AST)

AST levels determination is available at Quest Diagnostics, Clinical Trials, Quest House, 125-135 Staines Road, Hounslow, Middlesex, TW3 3JB, United Kingdom under Catalogue n#84450. For Study 3, the data are obtained at at BARC Europe, 3B,Industrie Park, Zwijnaarde, B-9052 Ghent, Belgium.

AST catalyzes the transamination of aspartate and 2-oxoglutarate, forming L-glutamate and oxalacetate. The oxalacetate is reduced to L-malate by malate dehydrogenase, whilst NADH is converted to NAD+, measured spectrophotometrically. Guidelines for the AST value have been issued by the association for Clinical Biochemistry and Laboratory Medicine, 130-132 Tooley Street LONDON SE1 2TU, united Kingdom, wherein the AST value should be below 34 U/l in Female and below 45 U/L in male. As disclosed in the respective study protocols for Study 1 or 2, at week 12, depending on the outcome of their treatment, the subjects may be continued in their initial treatment course, or reassigned to another treatment group in a randomized blinded fashion until week 24. Therefore, the number of subjects (N) for the period of either 12 weeks, or 24 weeks is provided to reflect this redistribution at week 12.

Namely, in Study 1, at Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d.

In Study 2, at Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24.

TABLE III
Study 1 - AST Mean CFB (IU/L) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 0 | 1 | 3 | 3 | 0 | 1 | 1 |
| 2 | 0 | 0 | 1 | 2 | 0 | 0 | 2 |
| 4 | 0 | 0 | 2 | 3 | 0 | 2 | 3 |
| 8 | 0 | 0 | 3 | 5 | 0 | 2 | 2 |
| 12 | −1 | 1 | 2 | 5 | 2 | 2 | 3 |

TABLE IV
Study 1 - AST Mean percentage CFB (%) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 2 | 7 | 14 | 16 | 3 | 6 | 10 |
| 2 | 2 | 5 | 9 | 13 | 6 | 6 | 14 |
| 4 | 4 | 5 | 11 | 19 | 6 | 14 | 19 |
| 8 | 3 | 7 | 17 | 27 | 10 | 14 | 17 |
| 12 | 0 | 10 | 16 | 29 | 14 | 17 | 20 |

TABLE V
Study 1 - AST Mean CFB (IU/L) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | −0.3 | −0.5 | 0.6 | 0.0 | −1.4 | −0.3 | −1.2 | −0.9 |
| | (N = 56) | | | | | | | | | |
| | Switched to | 0.0 | 1.7 | 1.2 | −1.1 | −0.8 | −0.6 | 1.3 | 1.2 | 3.2 |
| | 100 mg q.d. at | | | | | | | | | |
| | week 12 (N = 15) | | | | | | | | | |
| | Switched to | 0.0 | −0.1 | −0.3 | 0.7 | 0.3 | 1.1 | 2.1 | −0.1 | 3.5 |
| | 50 mg b.i.d. at | | | | | | | | | |
| | week 12 (N = 15) | | | | | | | | | |
| Continued | 50 mg q.d. | 0.0 | 0.8 | −0.2 | −0.4 | 0.4 | 0.6 | 1.4 | 2.1 | 1.0 |
| q.d. | N = 63 | | | | | | | | | |
| dosage | 100 mg q.d. | 0.0 | 3.0 | 1.0 | 1.6 | 2.8 | 2.4 | 1.5 | 2.6 | 4.4 |
| regimen | (N = 85) | | | | | | | | | |
| over 24 | 200 mg q.d. | 0.0 | 3.1 | 1.9 | 2.7 | 4.5 | 4.8 | 6.8 | 4.0 | 5.2 |
| weeks | (N = 86) | | | | | | | | | |
| Continued | 25 mg b.i.d. | 0.0 | 0.7 | 0.7 | 1.0 | 1.6 | 2.2 | 1.4 | 1.1 | 1.7 |
| b.i.d. | (N = 69) | | | | | | | | | |
| dosage | 50 mg b.i.d. | 0.0 | 0.5 | 0.4 | 1.8 | 1.7 | 2.4 | 1.9 | 3.4 | 1.5 |
| regimen | (N = 85) | | | | | | | | | |
| over 24 | 100 mg b.i.d. | 0.0 | 1.4 | 2.3 | 2.6 | 1.9 | 2.5 | 3.5 | 3.0 | 5.0 |
| weeks | (N = 84) | | | | | | | | | |
| Increased | from 50 mg | 0.0 | 0.3 | 0.0 | −0.5 | −0.4 | 0.1 | −0.9 | 0.4 | −1.7 |
| dosage | q.d. to 100 mg | | | | | | | | | |
| regimen | q.d. (N = 19) | | | | | | | | | |
| at 12 | from 25 mg | 0.0 | −5.0 | −2.3 | −2.9 | −4.1 | −1.0 | −3.3 | −0.6 | 0.6 |
| weeks | b.i.d. to 50 mg | | | | | | | | | |
| | b.i.d. (N = 17) | | | | | | | | | |

TABLE VI
Study 1 - AST Mean percentage CFB (%) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | −0.2 | −0.6 | 5.4 | 5.0 | −1.0 | 3.4 | −0.4 | −0.8 |
| | (N = 56) | | | | | | | | | |
| | Switched to | 0.0 | 9.9 | 9.6 | −3.2 | −2.5 | −1.4 | 1.0 | 9.8 | 23.1 |
| | 100 mg q.d. at | | | | | | | | | |
| | week 12 (N = 15) | | | | | | | | | |
| | Switched to | 0.0 | 4.5 | 2.2 | 6.8 | 2.2 | 6.4 | 14.9 | 1.9 | 19.7 |
| | 50 mg b.i.d. at | | | | | | | | | |
| | week 12 (N = 15) | | | | | | | | | |
| Continued | 50 mg q.d. | 0.0 | 7.5 | 5.7 | 5.2 | 9.0 | 11.4 | 15.1 | 16.8 | 12.9 |
| q.d. | (N = 63) | | | | | | | | | |
| dosage | 100 mg q.d. | 0.0 | 13.5 | 9.4 | 11.2 | 17.1 | 15.7 | 11.5 | 19.2 | 28.6 |
| regimen | (N = 85) | | | | | | | | | |
| over 24 | 200 mg q.d. | 0.0 | 16.1 | 12.8 | 18.7 | 27.2 | 28.8 | 36.8 | 23.3 | 30.6 |
| weeks | (N = 86) | | | | | | | | | |
| Continued | 25 mg b.i.d. | 0.0 | 6.7 | 7.1 | 8.6 | 14.0 | 16.4 | 12.6 | 10.8 | 14.0 |
| b.i.d. | (N = 69) | | | | | | | | | |
| dosage | 50 mg b.i.d. | 0.0 | 5.7 | 5.5 | 14.4 | 14.0 | 17.1 | 13.7 | 20.7 | 11.1 |
| regimen | N = 85 | | | | | | | | | |
| over 24 | 100 mg b.i.d. | 0.0 | 10.0 | 14.1 | 19.1 | 17.4 | 20.1 | 25.2 | 23.1 | 37.6 |
| weeks | (N = 84) | | | | | | | | | |
| Increased | from 50 mg | 0.0 | 5.0 | 3.4 | 3.6 | 3.0 | 4.2 | −0.2 | 4.9 | −2.5 |
| dosage | q.d. to 100 mg | | | | | | | | | |
| regimen | q.d. (N = 19) | | | | | | | | | |
| at 12 | from 25 mg | 0.0 | −12.5 | −0.7 | −2.7 | −7.2 | 4.5 | −2.3 | 8.2 | 14.2 |
| weeks | b.i.d. to 50 mg | | | | | | | | | |
| | b.i.d. (N = 17) | | | | | | | | | |

TABLE VII
Study 2 - AST Mean CFB (IU/L) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.0 | 0.0 | 0.0 | 0.0 |
| 1 | −0.7 | −1.8 | 0.4 | 0.1 |
| 2 | 0.1 | −2.0 | 1.3 | 0.9 |
| 4 | 0.0 | −1.7 | 1.3 | 0.8 |
| 8 | 0.2 | −2.5 | 1.9 | 1.4 |
| 12 | −0.7 | −0.2 | 1.9 | 1.1 |

TABLE VIII
Study 2 - AST Mean percentage CFB (%) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | −0.69 | 0.54 | 8.99 | 7.59 |
| 2 | 4.29 | 1.54 | 9.02 | 11.22 |
| 4 | 3.76 | −0.21 | 15.05 | 13.13 |
| 8 | 4.66 | −0.42 | 18.05 | 16.67 |
| 12 | −1.06 | 2.69 | 16.02 | 15.21 |

TABLE IX
Study 2 - AST Mean CFB (IU/L) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.0 | −0.7 | 0.1 | 0.0 | 0.2 | −0.7 | 0.9 | 2.2 | 2.1 |
| 100 mg q.d. (N = 72) | | | | | | | | | |
| 50 mg q.d. responders | 0.0 | −2.1 | −2.6 | −2.3 | −3.2 | −0.5 | 0.5 | 0.5 | −0.5 |
| (N = 57) | | | | | | | | | |
| 50 mg q.d. non | 0.0 | −0.4 | 0.3 | 0.3 | 0.0 | 0.8 | 0.8 | 1.8 | 2.6 |
| responders switching to | | | | | | | | | |
| 100 mg q.d. (N = 15) | | | | | | | | | |
| 100 mg q.d. (N = 70) | 0.0 | 0.4 | 1.3 | 1.3 | 1.9 | 1.9 | 5.4 | 2.4 | 1.5 |
| 200 mg q.d. (N = 69) | 0.0 | 0.1 | 0.9 | 0.8 | 1.4 | 1.1 | 2.2 | 1.7 | 1.4 |

TABLE X
Study 2 - AST Mean percentage CFB (%) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | −0.69 | 4.29 | 3.76 | 4.66 | −1.06 | 8.60 | 16.10 | 13.64 |
| 100 mg q.d. (N = 72) | | | | | | | | | |
| 50 mg q.d. responders | 0.00 | 0.98 | 1.26 | −1.03 | −1.06 | 1.37 | 8.81 | 7.58 | 5.14 |
| (N = 57) | | | | | | | | | |
| 50 mg q.d. non | 0.00 | −1.15 | 2.57 | 2.84 | 1.89 | 7.36 | 5.82 | 13.66 | 17.42 |
| responders switching to | | | | | | | | | |
| 100 mg q.d. (N = 15) | | | | | | | | | |
| 100 mg q.d. (N = 70) | 0.00 | 8.99 | 9.02 | 15.05 | 18.05 | 16.02 | 23.15 | 23.16 | 15.75 |
| 200 mg q.d. (N = 69) | 0.00 | 7.59 | 11.22 | 13.13 | 16.67 | 15.21 | 21.14 | 17.43 | 17.74 |

TABLE XI
Study 3 - AST Mean CFB (IU/L) - 10 weeks results

| Weeks | Placebo (N = 44) | 200 mg q.d. (N = 130) |
|---|---|---|
| 0 | 0.0 | 0.0 |
| 2 | 0.6 | 3.0 |
| 4 | 1.0 | 1.5 |
| 6 | 0.8 | 1.3 |
| 10 | 2.5 | 5.4 |

TABLE XII
Study 3 - AST Mean percentage CFB (%) - 10 weeks results

| Weeks | Placebo (N = 44) | 200 mg q.d. (N = 130) |
|---|---|---|
| 0 | 0.00 | 0.00 |
| 2 | 11.2 | 20.1 |
| 4 | 13.6 | 13.4 |
| 6 | 12.1 | 12.0 |
| 10 | 30.4 | 38.7 |

TABLE XIII
Study 3 - AST Mean CFB (IU/L) - 20 weeks results

| Week 0 to | Week 10 to week | | | | | Week | Week | Week | Week |
|---|---|---|---|---|---|---|---|---|---|
| week 10 | 20 | Week 0 | Week 2 | Week 4 | Week 6 | 10 | 12 | 16 | 20 |
| Placebo | Continued | 0.0 | −0.09 | −0.67 | 1.24 | 4.07 | −0.93 | −2.38 | −1.92 |
| | placebo (N = 22) | | | | | | | | |
| | To 100 mg q.d. | 0.0 | 1.27 | 2.32 | 0.45 | 1.36 | 1.64 | 0.68 | 1.10 |
| | (N = 22) | | | | | | | | |
| 200 mg | Continued | 0.0 | 4.24 | 0.73 | 1.12 | 5.61 | 1.64 | 3.78 | 1.53 |
| q.d. | 200 mg q.d. | | | | | | | | |
| | (N = 77) | | | | | | | | |
| | To 100 mg q.d. | 0.0 | 1.50 | 3.28 | 1.40 | 3.90 | 1.13 | 2.82 | 8.15 |
| | (N = 30) | | | | | | | | |
| | To placebo | 0.0 | 0.96 | 1.96 | 1.87 | 7.17 | 2.87 | 2.62 | 3.14 |
| | (N = 23) | | | | | | | | |

TABLE XIV
Study 3 - AST Mean percentage CFB (%) - 20 weeks results

| Week 0 to | Week 10 to week | | | | | Week | Week | Week | Week |
|---|---|---|---|---|---|---|---|---|---|
| week 10 | 20 | Week 0 | Week 2 | Week 4 | Week 6 | 10 | 12 | 16 | 20 |
| Placebo | Continued | 0.0 | 11.04 | 7.42 | 15.17 | 55.64 | 8.30 | 1.06 | 7.28 |
| | placebo (N = 22) | | | | | | | | |
| | To 100 mg q.d. | 0.0 | 11.34 | 18.62 | 9.65 | 13.18 | 16.84 | 10.89 | 16.93 |
| | (N = 22) | | | | | | | | |
| 200 mg | Continued | 0.0 | 26.67 | 11.03 | 12.72 | 43.70 | 18.27 | 32.98 | 19.45 |
| q.d. | 200 mg q.d. | | | | | | | | |
| | (N = 77) | | | | | | | | |
| | To 100 mg q.d. | 0.0 | 11.88 | 19.48 | 10.05 | 23.93 | 10.30 | 20.91 | 50.50 |
| | (N = 30) | | | | | | | | |
| | To placebo | 0.0 | 8.86 | 13.24 | 12.17 | 45.50 | 14.82 | 16.62 | 21.09 |
| | (N = 23) | | | | | | | | |

### 3.2. Alanine Aminotransferase (ALT)

ALT levels determination is available at Quest Diagnostics, Clinical Trials, Quest House, 125-135 Staines Road, Hounslow, Middlesex, TW3 3JB, United Kingdom under Catalogue n#84460.

ALT catalyzes the exchange of the amino group of alanine with the oxogroup of 2-ketoglutarate to form pyruvate and glutamate. In the presence of lactic dehydrogenase, pyruvate reacts with NADH to form NAD+, measured spectrophotometrically. Guidelines for the ALT value have been issued by the association for Clinical Biochemistry and Laboratory Medicine, 130-132 Tooley Street LONDON SE1 2TU, united Kingdom, wherein the ALT value should be below 34 U/l in Female and below 52 U/L in male.

As disclosed in the respective study protocols for Study 1 or 2, at week 12, depending on the outcome of their treatment, the subjects may be continued in their initial treatment course, or reassigned to another treatment group in a randomized blinded fashion until week 24. Therefore, the number of subjects (N) for the period of either 12 weeks, or 24 weeks is provided to reflect this redistribution at week 12.

Namely, in Study 1, at Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d.

In Study 2, at Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24.

TABLE XV
Study 1 - ALT Mean CFB (IU/L) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 1 | 1 | 3 | 2 | −1 | 1 | 1 |
| 2 | 0 | −1 | 1 | 1 | −2 | 1 | 1 |
| 4 | 0 | −1 | 1 | 1 | −1 | 3 | 1 |
| 8 | −1 | 0 | 2 | 4 | 0 | 2 | 1 |
| 12 | −2 | 0 | 2 | 3 | 1 | 2 | 2 |

TABLE XVI
Study 1 - ALT Mean percentage CFB (%) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 6 | 5 | 14 | 11 | 4 | 8 | 9 |
| 2 | −2 | 0 | 9 | 16 | 2 | 13 | 10 |
| 4 | 2 | 4 | 9 | 15 | 9 | 24 | 20 |
| 8 | 2 | 9 | 15 | 31 | 11 | 20 | 25 |
| 12 | −5 | 12 | 16 | 34 | 21 | 28 | 26 |

TABLE XVII
Study 1 - ALT Mean CFB (IU/L) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | 0.7 | −0.7 | 0.4 | −0.2 | −2.3 | −1.4 | −1.8 | −1.8 |
| | (N = 56) | | | | | | | | | |
| | Switched to | 0.0 | 0.3 | −0.7 | −3.1 | −2.0 | −2.9 | −1.3 | 0.5 | −0.1 |
| | 100 mg q.d. at | | | | | | | | | |
| | week 12 (N = 15) | | | | | | | | | |
| | Switched to | 0.0 | 2.3 | 1.2 | 1.1 | 0.0 | 1.8 | 0.5 | 0.7 | 1.0 |
| | 50 mg b.i.d. at | | | | | | | | | |
| | week 12 (N = 15) | | | | | | | | | |
| Continued | 50 mg q.d. | 0.0 | 0.9 | −0.7 | −0.6 | 0.4 | 0.5 | 1.1 | 2.2 | 1.7 |
| q.d. | (N = 63) | | | | | | | | | |
| dosage | 100 mg q.d. | 0.0 | 3.0 | 0.9 | 0.6 | 1.7 | 1.6 | 0.7 | 1.8 | 5.1 |
| regimen | (N = 85) | | | | | | | | | |
| over 24 | 200 mg q.d. | 0.0 | 2.3 | 1.4 | 1.1 | 3.9 | 3.4 | 4.7 | 2.9 | 5.4 |
| weeks | (N = 86) | | | | | | | | | |
| Continued | 25 mg b.i.d. | 0.0 | 0.8 | −0.4 | 0.3 | 1.8 | 2.5 | 1.2 | 0.0 | 1.4 |
| b.i.d. | N = 69 | | | | | | | | | |
| dosage | 50 mg b.i.d. | 0.0 | 0.6 | 1.2 | 2.5 | 2.2 | 2.1 | 1.6 | 3.0 | 1.8 |
| regimen | (N = 85) | | | | | | | | | |
| over 24 | 100 mg b.i.d. | 0.0 | 0.5 | 0.8 | 0.8 | 0.6 | 1.7 | 3.2 | 2.8 | 4.0 |
| weeks | N = 84 | | | | | | | | | |
| Increased | from 50 mg | 0.0 | 0.4 | −1.4 | −0.3 | −0.2 | −0.1 | −2.3 | −1.7 | −3.0 |
| dosage | q.d. to 100 mg | | | | | | | | | |
| regimen | q.d. (N = 19) | | | | | | | | | |
| at 12 | from 25 mg | 0.0 | −7.8 | −7.3 | −5.6 | −8.3 | −6.4 | −7.9 | −5.4 | −4.8 |
| weeks | b.i.d. to 50 mg | | | | | | | | | |
| | b.i.d. (N = 17) | | | | | | | | | |

TABLE XVIII
Study 1 - ALT Mean percentage CFB (%) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | 4.9 | −4.0 | 4.4 | 5.4 | −7.4 | −3.1 | −4.5 | −3.0 |
| | (N = 56) | | | | | | | | | |
| | Switched to | 0.0 | 5.8 | 1.5 | −7.8 | −6.1 | −11.9 | 0.4 | 5.9 | 10.0 |
| | 100 mg q.d. at | | | | | | | | | |
| | week 12 (N = 15) | | | | | | | | | |
| | Switched to | 0.0 | 8.5 | 4.2 | 4.2 | −1.8 | 10.3 | 1.1 | 0.7 | 6.8 |
| | 50 mg b.i.d. at | | | | | | | | | |
| | week 12 (N = 15) | | | | | | | | | |
| Continued | 50 mg q.d. | 0.0 | 6.6 | 1.4 | 4.8 | 10.6 | 13.8 | 18.1 | 22.1 | 19.4 |
| q.d. | (N = 63) | | | | | | | | | |
| dosage | 100 mg q.d. | 0.0 | 13.6 | 8.8 | 9.1 | 15.1 | 15.6 | 9.5 | 19.8 | 39.5 |
| regimen | (N = 85) | | | | | | | | | |
| over 24 | 200 mg q.d. | 0.0 | 11.4 | 15.5 | 15.1 | 31.1 | 33.9 | 43.8 | 32.9 | 47.5 |
| weeks | (N = 86) | | | | | | | | | |
| Continued | 25 mg b.i.d. | 0.0 | 8.9 | 5.4 | 10.9 | 18.4 | 28.6 | 21.4 | 15.2 | 25.1 |
| b.i.d. | (N = 69) | | | | | | | | | |
| dosage | 50 mg b.i.d. | 0.0 | 8.5 | 13.0 | 24.2 | 20.3 | 27.6 | 20.1 | 26.2 | 18.7 |
| regimen | (N = 85) | | | | | | | | | |
| over 24 | 100 mg b.i.d. | 0.0 | 8.8 | 10.4 | 20.4 | 24.5 | 26.0 | 32.4 | 34.2 | 47.8 |
| weeks | (N = 84) | | | | | | | | | |
| Increased | from 50 mg | 0.0 | 0.9 | −6.5 | 2.2 | 3.6 | 5.2 | −9.8 | −3.7 | −8.7 |
| dosage | q.d. to 100 mg | | | | | | | | | |
| regimen | q.d. (N = 19) | | | | | | | | | |
| at 12 | from 25 mg | 0.0 | −16.4 | −12.0 | 0.0 | −16.4 | −5.8 | −9.7 | 5.9 | 5.7 |
| weeks | b.i.d. to 50 mg | | | | | | | | | |
| | b.i.d. (N = 17) | | | | | | | | | |

TABLE XIX
Study 2 - ALT Mean CFB (IU/L) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.0 | 0.0 | 0.0 | 0.0 |
| 1 | −0.3 | −1.4 | 0.4 | −1.8 |
| 2 | −0.5 | −2.6 | 0.7 | −1.1 |
| 4 | 0.1 | −2.2 | 0.3 | −1.4 |
| 8 | −0.2 | −3.0 | −0.5 | −0.8 |
| 12 | −0.8 | −1.4 | 1.3 | −1.1 |

TABLE XX
Study 2 - ALT Mean percentage CFB (%) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | 4.34 | −1.29 | 13.98 | −0.92 |
| 2 | 5.32 | −3.25 | 13.25 | 1.07 |
| 4 | 10.86 | −3.73 | 17.82 | 4.71 |
| 8 | 8.36 | −0.73 | 7.83 | 8.41 |
| 12 | 4.01 | 1.77 | 14.61 | 5.69 |

TABLE XXI
Study 2 - ALT Mean CFB (IU/L) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.0 | −0.3 | −0.5 | 0.1 | −0.2 | −0.8 | 0.4 | 0.8 | 0.9 |
| 100 mg q.d. (N = 72) | | | | | | | | | |
| 50 mg q.d. responders | 0.0 | −1.7 | −3.3 | −3.0 | −3.4 | −1.6 | −0.2 | −0.5 | −0.2 |
| (N = 57) | | | | | | | | | |
| 50 mg q.d. non | 0.0 | −0.1 | −0.2 | 0.6 | −1.3 | −0.9 | 0.9 | 0.3 | 3.2 |
| responders switching to | | | | | | | | | |
| 100 mg q.d. (N = 15) | | | | | | | | | |
| 100 mg q.d. (N = 70) | 0.0 | 0.4 | 0.7 | 0.3 | −0.5 | 1.3 | 4.1 | 1.8 | 0.4 |
| 200 mg q.d. (N = 69) | 0.0 | −1.8 | −1.1 | −1.4 | −0.8 | −1.1 | 0.4 | −0.6 | −1.3 |

TABLE XXII
Study 2 - ALT Mean percentage CFB (%) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | 4.34 | 5.32 | 10.86 | 8.36 | 4.01 | 15.03 | 17.28 | 18.10 |
| 100 mg q.d. (N = 72) | | | | | | | | | |
| 50 mg q.d. responders | 0.00 | −1.89 | −3.98 | −5.93 | 0.30 | 2.63 | 17.03 | 8.38 | 16.54 |
| (N = 57) | | | | | | | | | |
| 50 mg q.d. non | 0.00 | 0.97 | −0.54 | 4.48 | −4.46 | −1.26 | 6.27 | 4.86 | 22.47 |
| responders switching to | | | | | | | | | |
| 100 mg q.d. (N = 15) | | | | | | | | | |
| 100 mg q.d. (N = 70) | 0.00 | 13.98 | 13.25 | 17.82 | 7.83 | 14.61 | 25.66 | 37.63 | 20.33 |
| 200 mg q.d. (N = 69) | 0.00 | −0.92 | 1.07 | 4.71 | 8.41 | 5.69 | 17.47 | 6.69 | 5.83 |

TABLE XXIII
Study 3 - ALT Mean CFB (IU/L) - 10 weeks results

| Weeks | Placebo (N = 44) | 200 mg q.d. (N = 130) |
|---|---|---|
| 0 | 0.0 | 0.0 |
| 2 | 1.6 | 0.0 |
| 4 | 2.0 | −0.6 |
| 6 | −0.1 | −1.2 |
| 10 | 0.6 | 1.6 |

TABLE XXIV
Study 3 - ALT Mean percentage CFB (%) - 10 weeks results

| Weeks | Placebo (N = 44) | 200 mg q.d. (N = 130) |
|---|---|---|
| 0 | 0.00 | 0.00 |
| 2 | 14.2 | 8.3 |
| 4 | 17.3 | 5.8 |
| 6 | 11.1 | 3.1 |
| 10 | 14.8 | 24.4 |

TABLE XXV
Study 3 - ALT Mean CFB (IU/L) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 0.82 | 0.17 | 0.59 | 1.40 | −1.00 | −2.92 | −2.08 |
| | placebo (N = 22) | | | | | | | | |
| | To 100 mg q.d. | 0.0 | 2.45 | 3.45 | −0.55 | 0.00 | −0.55 | −1.95 | −2.05 |
| | (N = 22) | | | | | | | | |
| 200 mg | Continued | 0.0 | 1.46 | 0.36 | −0.17 | 3.75 | 3.00 | 4.04 | 2.53 |
| q.d. | 200 mg q.d. | | | | | | | | |
| | (N = 77) | | | | | | | | |
| | To 100 mg q.d. | 0.0 | −2.03 | −1.93 | −1.60 | −0.50 | 0.03 | 0.21 | 6.15 |
| | (N = 30) | | | | | | | | |
| | To placebo | 0.0 | −2.30 | −2.04 | −3.78 | −1.48 | 1.48 | 1.90 | −0.38 |
| | (N = 23) | | | | | | | | |

TABLE XXVI
Study 3 - ALT Mean percentage CFB (%) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 8.26 | 9.48 | 13.76 | 20.69 | 7.51 | −3.32 | −0.87 |
| | placebo (N = 22) | | | | | | | | |
| | To 100 mg q.d. | 0.0 | 20.19 | 23.65 | 9.03 | 10.78 | 10.24 | 2.75 | 4.96 |
| | (N = 22) | | | | | | | | |
| 200 mg | Continued | 0.0 | 14.82 | 8.36 | 5.47 | 38.09 | 26.61 | 33.15 | 22.15 |
| q.d. | 200 mg q.d. | | | | | | | | |
| | (N = 77) | | | | | | | | |
| | To 100 mg q.d. | 0.0 | −4.84 | −3.24 | −0.60 | 5.92 | 3.56 | 8.83 | 38.62 |
| | (N = 30) | | | | | | | | |
| | To placebo | 0.0 | 3.63 | 8.87 | 1.03 | 12.60 | 16.15 | 23.87 | 17.82 |
| | (N = 23) | | | | | | | | |

### 3.3. Creatinine

Creatinine levels determination is available at Quest Diagnostics, Clinical Trials, Quest House, 125-135 Staines Road, Hounslow, Middlesex, TW3 3JB, United Kingdom under Catalogue n# 82565. For Study 3, the data are obtained at at BARC Europe, 3B,Industrie Park, Zwijnaarde, B-9052 Ghent, Belgium.

The measurement of creatinine in serum or in urine may be useful in the assessment of renal function.

The Enzymatic method is based on the determination of sarcosine after conversion of creatinine with the aid of creatininase, creatinase, and sarcosine oxidase. The liberated hydrogen peroxide reacts with 4-aminophenazone and HTIB to form a quinone imine chromogen. The reaction is catalyzed by peroxidase. The color intensity is directly proportional to concentration of creatinine present and can be measured photometrically.

Guidelines for the Creatinine value have been issued by the association for Clinical Biochemistry and Laboratory Medicine, 130-132 Tooley Street LONDON SE1 2TU, united Kingdom, wherein the creatinine concentration should be around 60-120 μmol/L in males, and 55-100 μmol/L (females) using Jaffe-based methods.

As disclosed in the respective study protocols for Study 1 or 2, at week 12, depending on the outcome of their treatment, the subjects may be continued in their initial treatment course, or reassigned to another treatment group in a randomized blinded fashion until week 24. Therefore, the number of subjects (N) for the period of either 12 weeks, or 24 weeks is provided to reflect this redistribution at week 12.

Namely, in Study 1, at Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d.

In Study 2, at Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24.

TABLE XXVII
Study 1 - Creatinine Mean CFB (μmol/L) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | −1 | 1 | 2 | 3 | 1 | 3 | 5 |
| 2 | 0 | 1 | 3 | 3 | 1 | 4 | 4 |
| 4 | 0 | 1 | 2 | 4 | 0 | 3 | 4 |
| 8 | 1 | 2 | 2 | 4 | 2 | 3 | 7 |
| 12 | 0 | 2 | 4 | 4 | 1 | 4 | 6 |

TABLE XXVIII
Study 1 - Creatinine Mean percentage CFB (%) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | −1 | 3 | 4 | 6 | 2 | 5 | 8 |
| 2 | 1 | 2 | 5 | 6 | 2 | 6 | 8 |
| 4 | 0 | 3 | 4 | 7 | 2 | 5 | 8 |
| 8 | 2 | 4 | 4 | 7 | 3 | 6 | 12 |
| 12 | 1 | 3 | 7 | 8 | 3 | 7 | 12 |

TABLE XXIX
Study 1 - Creatinine Mean CFB (μmol/L) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | −1.6 | 0.6 | 0.1 | 0.9 | 0.1 | 0.4 | 0.0 | −1.7 |
| | N = 56 | | | | | | | | | |
| | Switched to | 0.0 | 0.5 | −0.7 | −0.9 | −2.0 | −1.9 | 3.0 | 1.9 | 3.0 |
| | 100 mg q.d. at | | | | | | | | | |
| | week 12 (N = 15) | | | | | | | | | |
| | Switched to | 0.0 | 0.1 | 0.9 | 0.5 | 2.4 | 0.3 | 4.2 | 3.6 | 4.6 |
| | 50 mg b.i.d. at | | | | | | | | | |
| | week 12 (N = 15) | | | | | | | | | |
| Continued | 50 mg q.d. | 0.0 | 1.4 | 0.7 | 1.1 | 2.0 | 1.9 | 2.6 | 2.9 | 0.6 |
| q.d. | (N = 63) | | | | | | | | | |
| dosage | 100 mg q.d. | 0.0 | 2.3 | 2.5 | 2.0 | 1.9 | 3.8 | 2.3 | 2.9 | 1.5 |
| regimen | N = (85) | | | | | | | | | |
| over 24 | 200 mg q.d. | 0.0 | 3.0 | 3.1 | 3.7 | 4.1 | 4.1 | 4.8 | 4.6 | 3.2 |
| weeks | (N = 86) | | | | | | | | | |
| Continued | 25 mg b.i.d. | 0.0 | 1.2 | 1.3 | 0.7 | 1.5 | 1.3 | 1.8 | 1.7 | 0.5 |
| b.i.d. | N = 69 | | | | | | | | | |
| dosage | 50 mg b.i.d. | 0.0 | 2.9 | 3.6 | 2.9 | 3.5 | 3.8 | 4.7 | 5.7 | 2.8 |
| regimen | (N = 85) | | | | | | | | | |
| over 24 | 100 mg b.i.d. | 0.0 | 4.6 | 4.1 | 4.3 | 6.6 | 6.3 | 6.5 | 7.0 | 6.1 |
| weeks | (N = 84) | | | | | | | | | |
| Increased | from 50 mg | 0.0 | 0.7 | 1.8 | 2.5 | 2.6 | 0.6 | 4.5 | 2.9 | 2.2 |
| dosage | q.d. to 100 mg | | | | | | | | | |
| regimen | q.d. (N = 19) | | | | | | | | | |
| at 12 | from 25 mg | 0.0 | 0.4 | 0.2 | −0.6 | 1.8 | 0.2 | 3.8 | 1.7 | 0.5 |
| weeks | b.i.d. to 50 mg | | | | | | | | | |
| | b.i.d. (N = 17) | | | | | | | | | |

TABLE XXX
Study 1 - Creatinine Mean percentage CFB (%) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | −2.0 | 1.1 | 0.4 | 2.1 | 1.3 | 2.1 | 1.2 | −1.5 |
| | N = 56 | | | | | | | | | |
| | Switched to | 0.0 | 1.4 | −0.3 | −1.4 | −2.7 | −2.4 | 6.4 | 4.3 | 5.9 |
| | 100 mg q.d. at | | | | | | | | | |
| | week 12 (N = 15) | | | | | | | | | |
| | Switched to | 0.0 | 0.6 | 1.6 | 1.5 | 4.6 | 1.3 | 8.2 | 7.2 | 8.7 |
| | 50 mg b.i.d. at | | | | | | | | | |
| | week 12 (N = 15) | | | | | | | | | |
| Continued | 50 mg q.d. | 0.0 | 3.0 | 1.6 | 2.8 | 4.4 | 3.7 | 5.1 | 5.7 | 2.1 |
| q.d. | (N = 63) | | | | | | | | | |
| dosage | 100 mg q.d. | 0.0 | 4.2 | 4.9 | 3.5 | 3.8 | 6.8 | 4.8 | 5.4 | 3.8 |
| regimen | (N = 85) | | | | | | | | | |
| over 24 | 200 mg q.d. | 0.0 | 5.7 | 5.8 | 7.0 | 7.4 | 7.7 | 8.7 | 8.5 | 6.0 |
| weeks | (N = 86) | | | | | | | | | |
| Continued | 25 mg b.i.d. | 0.0 | 2.5 | 2.9 | 2.1 | 3.3 | 3.6 | 4.3 | 4.3 | 2.0 |
| b.i.d. | (N = 69) | | | | | | | | | |
| dosage | 50 mg b.i.d. | 0.0 | 5.5 | 6.0 | 5.4 | 6.4 | 6.6 | 8.2 | 9.6 | 5.5 |
| regimen | (N = 85) | | | | | | | | | |
| over 24 | 100 mg b.i.d. | 0.0 | 8.2 | 7.9 | 8.3 | 12.1 | 11.6 | 12.1 | 12.7 | 11.5 |
| weeks | (N = 84) | | | | | | | | | |
| Increased | from 50 mg | 0.0 | 1.5 | 3.0 | 3.9 | 4.1 | 1.9 | 8.0 | 5.2 | 3.7 |
| dosage | q.d. to 100 mg | | | | | | | | | |
| regimen | q.d. (N = 19) | | | | | | | | | |
| at 12 | from 25 mg | 0.0 | 0.8 | 0.3 | −0.4 | 3.9 | 0.4 | 7.6 | 4.8 | 1.4 |
| weeks | b.i.d. to 50 mg | | | | | | | | | |
| | b.i.d. (N = 17) | | | | | | | | | |

TABLE XXXI
Study 2 - Creatinine Mean CFB (μmol/L) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | −0.76 | 0.42 | 1.19 | 4.68 |
| 2 | −0.33 | 1.25 | 1.48 | 4.99 |
| 4 | −0.46 | 1.32 | 1.10 | 4.28 |
| 8 | 0.12 | 2.20 | 3.19 | 4.82 |
| 12 | 0.16 | 3.16 | 1.75 | 3.80 |

TABLE XXXII
Study 2 - Creatinine Mean percentage CFB (%) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | −0.49 | 1.10 | 3.00 | 8.07 |
| 2 | 0.18 | 2.59 | 3.21 | 8.56 |
| 4 | −0.13 | 3.01 | 3.23 | 7.37 |
| 8 | 0.74 | 4.34 | 5.79 | 8.70 |
| 12 | 1.14 | 6.02 | 3.96 | 7.96 |

TABLE XXXIII
Study 2 - Creatinine Mean CFB (μmol/L) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.0 | −0.8 | −0.3 | −0.5 | 0.1 | 0.2 | 1.9 | 2.7 | 4.6 |
| 100 mg q.d. (N = 72) | | | | | | | | | |
| 50 mg q.d. responders | 0.0 | 0.2 | 1.0 | 1.5 | 1.1 | 4.0 | 1.9 | 3.6 | 1.6 |
| (N = 57) | | | | | | | | | |
| 50 mg q.d. non | 0.0 | 1.1 | 2.0 | 0.6 | 6.2 | 0.0 | 5.9 | 4.5 | 5.2 |
| responders switching | | | | | | | | | |
| to 100 mg q.d. | | | | | | | | | |
| (N = 15) | | | | | | | | | |
| 100 mg q.d. (N = 70) | 0.0 | 1.2 | 1.5 | 1.1 | 3.2 | 1.7 | 2.9 | 2.3 | 2.0 |
| 200 mg q.d. (N = 69) | 0.0 | 4.7 | 5.0 | 4.3 | 4.8 | 3.8 | 5.3 | 6.6 | 3.5 |

TABLE XXXIV
Study 2 - Creatinine Mean percentage CFB (%) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | −0.49 | 0.18 | −0.13 | 0.74 | 1.14 | 3.61 | 5.14 | 8.41 |
| 100 mg q.d. (N = 72) | | | | | | | | | |
| 50 mg q.d. responders | 0.00 | 0.86 | 2.27 | 3.40 | 2.67 | 7.51 | 3.91 | 7.19 | 3.16 |
| (N = 57) | | | | | | | | | |
| 50 mg q.d. non | 0.00 | 2.03 | 3.77 | 1.58 | 10.36 | 0.74 | 11.14 | 8.07 | 9.28 |
| responders switching to | | | | | | | | | |
| 100 mg q.d. (N = 15) | | | | | | | | | |
| 100 mg q.d. (N = 70) | 0.00 | 3.00 | 3.21 | 3.23 | 5.79 | 3.96 | 6.44 | 4.63 | 4.17 |
| 200 mg q.d. (N = 69) | 0.00 | 8.07 | 8.56 | 7.37 | 8.70 | 7.96 | 9.81 | 13.11 | 8.41 |

TABLE XXXV
Study 3 - Creatinine Mean CFB (IU/L) - 10 weeks results

| Weeks | Placebo (N = 44) | 200 mg q.d. (N = 130) |
|---|---|---|
| 0 | 0.0 | 0.0 |
| 2 | 1.97 | 2.44 |
| 4 | 2.83 | 4.16 |
| 6 | 1.70 | 2.81 |
| 10 | 3.99 | 6.08 |

TABLE XXXVI
Study 3 - Creatinine Mean percentage CFB (%) - 10 weeks results

| Weeks | Placebo (N = 44) | 200 mg q.d. (N = 130) |
|---|---|---|
| 0 | 0.00 | 0.00 |
| 2 | 3.57 | 4.41 |
| 4 | 4.91 | 6.55 |
| 6 | 2.49 | 4.82 |
| 10 | 6.11 | 10.05 |

TABLE XXXVII
Study 3 - Creatinine Mean CFB (μmol/L) - 20 weeks results

| Week 0 to | Week 10 to week | | | | | Week | Week | Week | Week |
|---|---|---|---|---|---|---|---|---|---|
| week 10 | 20 | Week 0 | Week 2 | Week 4 | Week 6 | 10 | 12 | 16 | 20 |
| Placebo | Continued | 0.0 | 1.49 | 2.39 | 1.09 | 2.54 | 3.70 | 3.59 | 3.75 |
| | placebo (N = 22) | | | | | | | | |
| | To 100 mg q.d. | 0.0 | 2.45 | 3.18 | 2.17 | 4.98 | 3.09 | 4.33 | 3.11 |
| | (N = 22) | | | | | | | | |
| 200 mg | Continued | 0.0 | 2.22 | 3.93 | 2.51 | 6.00 | 4.02 | 1.81 | 3.20 |
| q.d. | 200 mg q.d. | | | | | | | | |
| | (N = 77) | | | | | | | | |
| | To 100 mg q.d. | 0.0 | 3.36 | 4.98 | 4.00 | 6.48 | 2.92 | 4.89 | 3.43 |
| | (N = 30) | | | | | | | | |
| | To placebo | 0.0 | 1.95 | 3.83 | 2.15 | 5.88 | 1.84 | 0.47 | −0.26 |
| | (N = 23) | | | | | | | | |

TABLE XXXVIII
Study 3 - Creatinine Mean percentage CFB (%) - 20 weeks results

| Week 0 to | Week 10 to week | | | | | Week | Week | Week | Week |
|---|---|---|---|---|---|---|---|---|---|
| week 10 | 20 | Week 0 | Week 2 | Week 4 | Week 6 | 10 | 12 | 16 | 20 |
| Placebo | Continued | 0.0 | 2.95 | 4.38 | 1.31 | 3.15 | 4.70 | 5.50 | 6.18 |
| | placebo (N = 22) | | | | | | | | |
| | To 100 mg q.d. | 0.0 | 4.19 | 5.33 | 3.40 | 8.12 | 4.87 | 6.52 | 4.31 |
| | (N = 22) | | | | | | | | |
| 200 mg | Continued | 0.0 | 4.13 | 6.08 | 4.44 | 9.34 | 7.10 | 3.62 | 5.43 |
| q.d. | 200 mg q.d. | | | | | | | | |
| | (N = 77) | | | | | | | | |
| | To 100 mg q.d. | 0.0 | 5.77 | 8.03 | 6.37 | 11.55 | 6.01 | 8.86 | 6.19 |
| | (N = 30) | | | | | | | | |
| | To placebo | 0.0 | 3.55 | 6.09 | 3.92 | 10.23 | 3.14 | 1.93 | 0.68 |
| | (N = 23) | | | | | | | | |

### 3.4. Hematology: Complete Blood Count (CBC)

CBC levels determination is available at Quest Diagnostics, Clinical Trials, Quest House, 125-135 Staines Road, Hounslow, Middlesex, TW3 3JB, United Kingdom under Catalogue n#85025. For Study 3, the data are obtained at at BARC Europe, 3B,Industrie Park, Zwijnaarde, B-9052 Ghent, Belgium.

The Coulter employs electronic counting and sizing of particles to quantitate and evaluate blood cells. The LH750/LH780 WBC Differential analysis and classification are based on simultaneous measuring of cell volume, high frequency conductivity and laser light scatter. The number of neutrophils; lymphocytes and platelets can be determined using these methods. Hemoglobin, released by hemolysis to a stable cyanide containing pigment , is measured by photometric absorbance.

As disclosed in the respective study protocols for Study 1 or 2, at week 12, depending on the outcome of their treatment, the subjects may be continued in their initial treatment course, or reassigned to another treatment group in a randomized blinded fashion until week 24. Therefore, the number of subjects (N) for the period of either 12 weeks, or 24 weeks is provided to reflect this redistribution at week 12.

Namely, in Study 1, at Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d.

In Study 2, at Week 12, all subjects on placebo and the subjects on the 50 mg dose who did not achieve at least 20% improvement in swollen joint count (SJC66) and tender joint count (TJC68) were assigned to 100 mg q.d. in a blinded fashion and continued treatment until Week 24. Subjects in the other groups maintained their randomized treatment until Week 24.

TABLE XXXIX
Study 1 - Hemoglobin Mean CFB (g/L) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | −2 | −2 | 0 | 0 | 1 | −1 | 1 |
| 2 | −3 | −2 | −1 | −1 | 0 | −2 | 1 |
| 4 | −2 | −1 | 1 | 2 | 0 | −1 | 1 |
| 8 | −2 | 0 | 1 | 4 | 2 | 1 | 4 |
| 12 | 0 | 1 | 2 | 4 | 3 | 2 | 4 |

TABLE XL
Study 1 - Hemoglobin Mean CFB (g/L) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | −1.7 | −3.1 | −1.6 | −2.1 | −0.2 | −0.7 | 1.9 | −0.8 |
| | (N = 56) | | | | | | | | | |
| | Switched to | 0.0 | −2.0 | −3.4 | −3.4 | −4.3 | −2.7 | 0.6 | 1.3 | 0.0 |
| | 100 mg q.d. at | | | | | | | | | |
| | week 12 (N = 15) | | | | | | | | | |
| | Switched to | 0.0 | −1.3 | −2.7 | −1.3 | 0.6 | 1.1 | 0.9 | 1.6 | 2.3 |
| | 50 mg b.i.d. at | | | | | | | | | |
| | week 12 (N = 15) | | | | | | | | | |
| Continued | 50 mg q.d. | 0.0 | −1.9 | −2.7 | −1.8 | −0.5 | 0.9 | 0.8 | 0.2 | 0.4 |
| q.d. | (N = 63) | | | | | | | | | |
| dosage | 100 mg q.d. | 0.0 | 0.2 | −0.7 | 0.6 | 1.2 | 2.4 | 2.8 | 2.9 | 3.3 |
| regimen | (N = 85) | | | | | | | | | |
| over 24 | 200 mg q.d. | 0.0 | −0.1 | −0.5 | 1.6 | 3.6 | 4.1 | 3.6 | 4.5 | 3.4 |
| weeks | (N = 86) | | | | | | | | | |
| Continued | 25 mg b.i.d. | 0.0 | 0.9 | 0.3 | 0.3 | 1.9 | 2.9 | 2.9 | 3.1 | 3.0 |
| b.i.d. | (N = 69) | | | | | | | | | |
| dosage | 50 mg b.i.d. | 0.0 | −0.9 | −2.2 | −0.8 | 0.5 | 1.5 | 2.6 | 2.5 | 1.4 |
| regimen | (N = 85) | | | | | | | | | |
| over 24 | 100 mg b.i.d. | 0.0 | 0.7 | 0.8 | 1.0 | 3.8 | 4.4 | 4.4 | 3.5 | 4.9 |
| weeks | (N = 84) | | | | | | | | | |
| Increased | from 50 mg | 0.0 | −0.7 | 0.3 | 0.8 | 1.2 | 1.8 | 1.7 | 3.2 | 1.2 |
| dosage | q.d. to 100 mg | | | | | | | | | |
| regimen | q.d. (N = 19) | | | | | | | | | |
| at 12 | from 25 mg | 0.0 | −0.2 | −2.4 | −0.4 | 0.6 | 3.6 | 3.3 | 3.2 | 3.9 |
| weeks | b.i.d. to 50 mg | | | | | | | | | |
| | b.i.d. (N = 17) | | | | | | | | | |

TABLE XLI
Study 1 - Hemoglobin Mean percentage CFB (%) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | −1 | −1 | 0 | 0 | 1 | −1 | 1 |
| 2 | −2 | −1 | 0 | 0 | 0 | −2 | 1 |
| 4 | −1 | −1 | 1 | 2 | 1 | −1 | 1 |
| 8 | −1 | 0 | 1 | 3 | 2 | 0 | 3 |
| 12 | 0 | 1 | 2 | 3 | 3 | 1 | 4 |

TABLE XLII
Study 1 - Hemoglobin Mean percentage CFB (%) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | −1.3 | −2.2 | −1.2 | −1.6 | −0.1 | −0.5 | 1.6 | −0.4 |
| | (N = 56) | | | | | | | | | |
| | Switched to | 0.0 | −1.5 | −2.7 | −2.7 | −3.3 | −2.0 | 0.5 | 1.1 | 0.1 |
| | 100 mg q.d. at | | | | | | | | | |
| | week 12 (N = 15) | | | | | | | | | |
| | Switched to | 0.0 | −1.0 | −2.0 | −0.9 | 0.7 | 1.0 | 0.8 | 1.4 | 1.8 |
| | 50 mg b.i.d. at | | | | | | | | | |
| | week 12 (N = 15) | | | | | | | | | |
| Continued | 50 mg q.d. | 0.0 | −1.4 | −2.0 | −1.3 | −0.4 | 0.9 | 0.7 | 0.4 | 0.6 |
| q.d. | (N = 63) | | | | | | | | | |
| dosage | 100 mg q.d. | 0.0 | 0.3 | −0.4 | 0.6 | 1.2 | 2.2 | 2.6 | 2.6 | 3.0 |
| regimen | (N = 85) | | | | | | | | | |
| over 24 | 200 mg q.d. | 0.0 | 0.1 | −0.2 | 1.5 | 3.0 | 3.5 | 3.1 | 3.8 | 2.9 |
| weeks | (N = 86) | | | | | | | | | |
| Continued | 25 mg b.i.d. | 0.0 | 1.0 | 0.5 | 0.7 | 1.9 | 2.9 | 2.9 | 3.0 | 3.0 |
| b.i.d. | (N = 69) | | | | | | | | | |
| dosage | 50 mg b.i.d. | 0.0 | −0.7 | −1.6 | −0.6 | 0.5 | 1.3 | 2.2 | 2.0 | 1.2 |
| regimen | (N = 85) | | | | | | | | | |
| over 24 | 100 mg b.i.d. | 0.0 | 0.6 | 0.8 | 1.0 | 3.2 | 3.7 | 3.8 | 3.1 | 4.3 |
| weeks | (N = 84) | | | | | | | | | |
| Increased | from 50 mg | 0.0 | −0.5 | 0.4 | 0.8 | 1.1 | 1.6 | 1.4 | 2.6 | 1.0 |
| dosage | q.d. to 100 mg | | | | | | | | | |
| regimen | q.d. (N = 19) | | | | | | | | | |
| at 12 | from 25 mg | 0.0 | −0.1 | −1.7 | −0.1 | 0.7 | 3.6 | 3.3 | 3.2 | 3.8 |
| weeks | b.i.d. to 50 mg | | | | | | | | | |
| | b.i.d. (N = 17) | | | | | | | | | |

TABLE XLIII
Study 1 - Neutrophils Mean CFB (giga/L) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0.0 | 0.0 | 0.0 | 0.0 | 0.0 | 0.0 | 0.0 |
| 1 | 0.0 | −0.5 | −0.2 | −0.4 | −0.1 | −0.5 | −1.1 |
| 2 | 0.0 | −0.4 | −0.3 | −0.7 | −0.3 | −0.7 | −1.2 |
| 4 | −0.1 | −0.7 | −0.6 | −1.2 | −0.2 | −0.6 | −1.6 |
| 8 | −0.1 | −0.6 | −0.5 | −1.2 | −0.4 | −1.0 | −1.6 |
| 12 | −0.1 | −1.0 | −0.6 | −1.1 | −0.6 | −0.8 | −1.6 |

TABLE XLIV
Study 1 - Neutrophils Mean CFB (giga/L) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | 0.1 | −0.1 | −0.1 | 0.0 | −0.1 | −0.2 | 0.1 | 0.2 |
| | (N = 56) | | | | | | | | | |
| | Switched to | 0.0 | −0.7 | −0.2 | −0.7 | −0.7 | −0.8 | −1.5 | −1.1 | −1.3 |
| | 100 mg q.d. at | | | | | | | | | |
| | week 12 (N = 15) | | | | | | | | | |
| | Switched to | 0.0 | 0.3 | 0.6 | 0.9 | 0.5 | 0.4 | −0.5 | −0.8 | −1.3 |
| | 50 mg b.i.d. at | | | | | | | | | |
| | week 12 (N = 15) | | | | | | | | | |
| Continued | 50 mg q.d. | 0.0 | −0.5 | −0.3 | −0.6 | −0.5 | −1.0 | −1.0 | −0.9 | −0.8 |
| q.d. | (N = 63) | | | | | | | | | |
| dosage | 100 mg q.d. | 0.0 | −0.2 | −0.3 | −0.6 | −0.5 | −0.6 | −0.5 | −0.5 | −0.7 |
| regimen | (N = 85) | | | | | | | | | |
| over 24 | 200 mg q.d. | 0.0 | −0.4 | −0.7 | −1.3 | −1.3 | −1.1 | −1.3 | −1.4 | −1.1 |
| weeks | (N = 86) | | | | | | | | | |
| Continued | 25 mg b.i.d. | 0.0 | −0.1 | −0.4 | −0.3 | −0.5 | −0.6 | −0.3 | −0.6 | −0.9 |
| b.i.d. | (N = 69) | | | | | | | | | |
| dosage | 50 mg b.i.d. | 0.0 | −0.5 | −0.7 | −0.6 | −1.0 | −0.8 | −1.0 | −1.3 | −1.1 |
| regimen | (N = 85) | | | | | | | | | |
| over 24 | 100 mg b.i.d. | 0.0 | −1.1 | −1.2 | −1.6 | −1.6 | −1.6 | −1.6 | −1.9 | −1.9 |
| weeks | (N = 84) | | | | | | | | | |
| Increased | from 50 mg | 0.0 | −0.3 | −1.0 | −1.2 | −1.0 | −0.9 | −1.5 | −0.9 | −0.8 |
| dosage | q.d. to 100 mg | | | | | | | | | |
| regimen | q.d. (N = 19) | | | | | | | | | |
| at 12 | from 25 mg | 0.0 | −0.3 | 0.0 | −0.2 | 0.1 | −0.7 | −0.8 | −0.4 | −0.3 |
| weeks | b.i.d. to 50 mg | | | | | | | | | |
| | b.i.d. (N = 17) | | | | | | | | | |

TABLE XLV
Study 1 - Neutrophils Mean percentage CFB (%) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 8 | −3 | 1 | −2 | 7 | −5 | −12 |
| 2 | 12 | −2 | −1 | −6 | 4 | −7 | −15 |
| 4 | 3 | −5 | −5 | −13 | 3 | −6 | −20 |
| 8 | 14 | −2 | −4 | −8 | 1 | −13 | −19 |
| 12 | 10 | −5 | −5 | −5 | −1 | −10 | −20 |

TABLE XLVI
Study 1 - Neutrophils Mean percentage CFB (%) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | 12.3 | 14.2 | 0.7 | 21.0 | 15.6 | 17.6 | 12.4 | 18.1 |
| | N = 56 | | | | | | | | | |
| | Switched to | 0.0 | −9.4 | 0.2 | −8.2 | −6.1 | −10.3 | −18.6 | −13.4 | −19.3 |
| | 100 mg q.d. at | | | | | | | | | |
| | week 12 (N = 15) | | | | | | | | | |
| | Switched to | 0.0 | 10.2 | 16.5 | 19.4 | 11.0 | 10.7 | 6.8 | 0.4 | −9.6 |
| | 50 mg b.i.d. at | | | | | | | | | |
| | week 12 (N = 15) | | | | | | | | | |
| Continued | 50 mg q.d. | 0.0 | −3.5 | −0.5 | −2.4 | −0.1 | −5.5 | −7.7 | −3.4 | −6.3 |
| q.d. | (N = 63) | | | | | | | | | |
| dosage | 100 mg q.d. | 0.0 | 1.0 | −0.9 | −4.7 | −3.7 | −5.1 | −2.7 | −2.4 | −6.6 |
| regimen | (N = 85) | | | | | | | | | |
| over 24 | 200 mg q.d. | 0.0 | −1.8 | −5.6 | −13.5 | −7.6 | −4.7 | −13.7 | −9.7 | −4.8 |
| weeks | (N = 86) | | | | | | | | | |
| Continued | 25 mg b.i.d. | 0.0 | 8.9 | 3.9 | 4.7 | 0.4 | 1.7 | 4.6 | 0.6 | −6.4 |
| b.i.d. | (N = 69) | | | | | | | | | |
| dosage | 50 mg b.i.d. | 0.0 | −4.6 | −7.2 | −6.0 | −12.9 | −10.0 | −12.1 | −16.4 | −13.1 |
| regimen | N = 85 | | | | | | | | | |
| over 24 | 100 mg b.i.d. | 0.0 | −12.3 | −14.9 | −20.1 | −19.2 | −20.1 | −18.7 | −23.0 | −25.3 |
| weeks | (N = 84) | | | | | | | | | |
| Increased | from 50 mg | 0.0 | −0.7 | −8.1 | −11.4 | −7.5 | −3.9 | −16.4 | −6.8 | −7.3 |
| dosage | q.d. to 100 mg | | | | | | | | | |
| regimen | q.d. (N = 19) | | | | | | | | | |
| at 12 | from 25 mg | 0.0 | −1.2 | 4.3 | −4.2 | 3.0 | −9.1 | −12.0 | −2.6 | −3.2 |
| weeks | b.i.d. to 50 mg | | | | | | | | | |
| | b.i.d. (N = 17) | | | | | | | | | |

TABLE XLVII
Study 1 - Platelets Mean CFB (giga/L) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 8 | 1 | −2 | 10 | 8 | 4 | 8 |
| 2 | 13 | −10 | −9 | −5 | 2 | −6 | −18 |
| 4 | 8 | −15 | −28 | −34 | −9 | −24 | −55 |
| 8 | 8 | −23 | −28 | −30 | −19 | −30 | −49 |
| 12 | 8 | −24 | −37 | −35 | −29 | −35 | −58 |

TABLE XLVIII
Study 1 - Platelets Mean CFB (giga/L) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | 4.2 | 4.6 | 6.4 | 8.9 | 4.2 | −1.1 | −4.1 | 7.5 |
| | (N = 56) | | | | | | | | | |
| | Switched to | 0.0 | 11.9 | 27.8 | 18.5 | −12.1 | 20.7 | 4.9 | −12.7 | −11.4 |
| | 100 mg q.d. at | | | | | | | | | |
| | week 12 (N = 15) | | | | | | | | | |
| | Switched to | 0.0 | 19.0 | 28.5 | 3.9 | 21.5 | 9.1 | −11.1 | −10.8 | −29.9 |
| | 50 mg b.i.d. at | | | | | | | | | |
| | week 12 (N = 15) | | | | | | | | | |
| Continued | 50 mg q.d. | 0.0 | 5.5 | −3.4 | −10.6 | −17.1 | −21.1 | −24.4 | −21.8 | −24.5 |
| q.d. | (N = 63) | | | | | | | | | |
| dosage | 100 mg q.d. | 0.0 | −1.9 | −9.2 | −28.4 | −27.9 | −36.7 | −33.7 | −45.8 | −48.4 |
| regimen | (N = 85) | | | | | | | | | |
| over 24 | 200 mg q.d. | 0.0 | 10.2 | −5.0 | −34.1 | −30.2 | −34.5 | −40.8 | −36.8 | −32.9 |
| weeks | (N = 86) | | | | | | | | | |
| Continued | 25 mg b.i.d. | 0.0 | 5.5 | −0.8 | −12.1 | −24.0 | −27.0 | −33.9 | −18.6 | −27.6 |
| b.i.d. | (N = 69) | | | | | | | | | |
| dosage | 50 mg b.i.d. | 0.0 | 3.7 | −6.1 | −23.7 | −30.1 | −35.4 | −35.0 | −36.2 | −35.0 |
| regimen | (N = 85) | | | | | | | | | |
| over 24 | 100 mg b.i.d. | 0.0 | 8.2 | −18.4 | −54.7 | −48.7 | −57.8 | −60.5 | −46.2 | −52.4 |
| weeks | (N = 84) | | | | | | | | | |
| Increased | from 50 mg q.d. | 0.0 | −16.3 | −29.5 | −28.9 | −40.3 | −32.8 | −33.9 | −47.4 | −42.9 |
| dosage | to 100 mg q.d. | | | | | | | | | |
| regimen | N = 19 | | | | | | | | | |
| at 12 | from 25 mg | 0.0 | 16.5 | 11.0 | 5.2 | 0.6 | −36.1 | −28.4 | −20.9 | −25.5 |
| weeks | b.i.d. to 50 mg | | | | | | | | | |
| | b.i.d. (N = 17) | | | | | | | | | |

TABLE XLIX
Study 1 - Platelets Mean percentage CFB (%) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 3 | 2 | 0 | 6 | 5 | 2 | 4 |
| 2 | 4 | −1 | −2 | 1 | 3 | −1 | −5 |
| 4 | 3 | −3 | −7 | −7 | 1 | −7 | −14 |
| 8 | 3 | −5 | −7 | −8 | −3 | −8 | −12 |
| 12 | 3 | −5 | −9 | −9 | −5 | −10 | −15 |

TABLE L
Study 1 - Platelets Mean percentage CFB (%) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | 1.3 | 1.4 | 2.7 | 3.4 | 1.8 | −0.5 | −1.2 | 3.2 |
| | (N = 56) | | | | | | | | | |
| | Switched to | 0.0 | 2.6 | 7.2 | 5.4 | −3.1 | 6.0 | 2.4 | −1.1 | −1.8 |
| | 100 mg q.d. at | | | | | | | | | |
| | week 12 (N = 15) | | | | | | | | | |
| | Switched to | 0.0 | 7.7 | 10.3 | 1.5 | 9.5 | 5.0 | 1.7 | −1.8 | −6.6 |
| | 50 mg b.i.d. at | | | | | | | | | |
| | week 12 (N = 15) | | | | | | | | | |
| Continued | 50 mg q.d. | 0.0 | 3.1 | 0.7 | −2.7 | −4.2 | −5.1 | −6.1 | −5.1 | −6.0 |
| q.d. | (N = 63) | | | | | | | | | |
| dosage | 100 mg q.d. | 0.0 | −0.3 | −1.8 | −6.5 | −6.9 | −8.5 | −7.3 | −11.1 | −12.0 |
| regimen | (N = 85) | | | | | | | | | |
| over 24 | 200 mg q.d. | 0.0 | 5.5 | 1.0 | −7.5 | −7.8 | −9.1 | −11.2 | −9.6 | −8.2 |
| weeks | (N = 86) | | | | | | | | | |
| Continued | 25 mg b.i.d. | 0.0 | 4.8 | 3.5 | 0.4 | −3.1 | −4.1 | −5.4 | −1.3 | −4.2 |
| b.i.d. | (N = 69) | | | | | | | | | |
| dosage | 50 mg b.i.d. | 0.0 | 2.1 | −1.0 | −6.7 | −8.0 | −9.7 | −9.6 | −10.4 | −10.0 |
| regimen | (N = 85) | | | | | | | | | |
| over 24 | 100 mg b.i.d. | 0.0 | 3.5 | −4.7 | −14.5 | −12.2 | −14.6 | −15.3 | −11.4 | −12.1 |
| weeks | (N = 84) | | | | | | | | | |
| Increased | from 50 mg | 0.0 | −1.8 | −4.6 | −4.9 | −8.7 | −4.3 | −7.1 | −9.0 | −7.6 |
| dosage | q.d. to 100 mg | | | | | | | | | |
| regimen | q.d. N = 19 | | | | | | | | | |
| at 12 | from 25 mg | 0.0 | 4.7 | 3.2 | 1.0 | −1.1 | −9.3 | −8.2 | −5.4 | −7.6 |
| weeks | b.i.d. to 50 mg | | | | | | | | | |
| | b.i.d. (N = 17) | | | | | | | | | |

TABLE LI
Study 1 - Lymphocytes Mean CFB (giga/L) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0.0 | 0.0 | 0.0 | 0.0 | 0.0 | 0.0 | 0.0 |
| 1 | −0.1 | 0.0 | 0.1 | 0.4 | 0.1 | 0.4 | 0.5 |
| 2 | −0.1 | 0.0 | 0.2 | 0.2 | 0.0 | 0.2 | 0.4 |
| 4 | 0.0 | 0.0 | 0.0 | 0.1 | 0.0 | 0.1 | 0.3 |
| 8 | 0.1 | 0.1 | −0.1 | 0.2 | 0.0 | 0.2 | 0.2 |
| 12 | 0.0 | 0.0 | −0.1 | 0.0 | 0.0 | 0.1 | 0.1 |

TABLE LII
Study 1 - Lymphocytes Mean CFB (giga/L) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | −0.1 | −0.1 | −0.1 | 0.0 | 0.0 | 0.0 | 0.0 | 0.0 |
| | (N = 56) | | | | | | | | | |
| | Switched to | 0.0 | −0.3 | 0.0 | 0.0 | −0.1 | −0.2 | 0.1 | −0.2 | 0.0 |
| | 100 mg q.d. at | | | | | | | | | |
| | week 12 | | | | | | | | | |
| | (N = 15) | | | | | | | | | |
| | Switched to | 0.0 | 0.1 | 0.1 | 0.2 | 0.4 | 0.2 | 0.1 | 0.1 | 0.4 |
| | 50 mg b.i.d. at | | | | | | | | | |
| | week 12 N = 15 | | | | | | | | | |
| Continued | 50 mg q.d. | 0.0 | 0.0 | 0.0 | 0.1 | 0.1 | 0.0 | 0.0 | 0.1 | −0.1 |
| q.d. | (N = 63) | | | | | | | | | |
| dosage | 100 mg q.d. | 0.0 | 0.1 | 0.2 | 0.0 | −0.1 | −0.1 | −0.1 | −0.1 | −0.1 |
| regimen | (N = 85) | | | | | | | | | |
| over 24 | 200 mg q.d. | 0.0 | 0.4 | 0.2 | 0.1 | 0.2 | 0.0 | 0.1 | 0.1 | 0.1 |
| weeks | (N = 86) | | | | | | | | | |
| Continued | 25 mg b.i.d. | 0.0 | 0.1 | 0.0 | 0.0 | 0.0 | 0.1 | 0.0 | 0.0 | 0.1 |
| b.i.d. | (N = 69) | | | | | | | | | |
| dosage | 50 mg b.i.d. | 0.0 | 0.4 | 0.2 | 0.1 | 0.2 | 0.1 | 0.1 | 0.1 | 0.0 |
| regimen | (N = 85) | | | | | | | | | |
| over 24 | 100 mg b.i.d. | 0.0 | 0.5 | 0.4 | 0.3 | 0.2 | 0.1 | 0.1 | 0.2 | 0.1 |
| weeks | (N = 84) | | | | | | | | | |
| Increased | from 50 mg | 0.0 | 0.1 | 0.1 | 0.0 | 0.2 | 0.2 | 0.1 | 0.1 | 0.0 |
| dosage | q.d. to 100 mg | | | | | | | | | |
| regimen | q.d. N = 19 | | | | | | | | | |
| at 12 | from 25 mg | 0.0 | 0.0 | 0.0 | −0.2 | −0.1 | 0.0 | 0.1 | 0.1 | −0.1 |
| weeks | b.i.d. to 50 mg | | | | | | | | | |
| | b.i.d. (N = 17) | | | | | | | | | |

TABLE LIII
Study 1 - Lymphocytes Mean percentage CFB (%) - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| Weeks | (N = 86) | (N = 82) | (N = 85) | (N = 86) | (N = 86) | (N = 85) | (N = 84) |
| 0 | 0 | 0 | 0 | 0 | 0 | 0 | 0 |
| 1 | 1 | 7 | 13 | 23 | 10 | 24 | 28 |
| 2 | 4 | 7 | 19 | 15 | 7 | 18 | 23 |
| 4 | 4 | 5 | 6 | 10 | 4 | 10 | 18 |
| 8 | 8 | 10 | 2 | 13 | 4 | 16 | 13 |
| 12 | 7 | 7 | 2 | 5 | 8 | 13 | 10 |

TABLE LIV
Study 1 - Lymphocytes Mean percentage CFB (%) - 24 weeks results

| Study | | Time points (weeks) | | | | | | | | |
|---|---|---|---|---|---|---|---|---|---|---|
| groups | Dosage | 0 | 1 | 2 | 4 | 8 | 12 | 16 | 20 | 24 |
| Placebo | Continued | 0.0 | 0.1 | −0.6 | 3.5 | 5.0 | 5.1 | 7.8 | 4.5 | 0.0 |
| | (N = 56) | | | | | | | | | |
| | Switched to | 0.0 | −0.1 | 13.0 | 10.9 | 8.4 | 10.2 | 18.3 | 2.4 | 13.6 |
| | 100 mg q.d. at | | | | | | | | | |
| | week 12 (N = 15) | | | | | | | | | |
| | Switched to | 0.0 | 5.0 | 12.1 | 12.9 | 20.5 | 12.6 | 8.6 | 12.1 | 23.9 |
| | 50 mg b.i.d. at | | | | | | | | | |
| | week 12 (N = 15) | | | | | | | | | |
| Continued | 50 mg q.d. | 0.0 | 6.3 | 6.9 | 5.3 | 9.5 | 4.5 | 2.5 | 8.5 | −0.8 |
| q.d. | (N = 63) | | | | | | | | | |
| dosage | 100 mg q.d. | 0.0 | 12.6 | 19.2 | 6.1 | 2.1 | 2.1 | −0.6 | 3.4 | −1.0 |
| regimen | N = 85 | | | | | | | | | |
| over 24 | 200 mg q.d. | 0.0 | 23.4 | 15.0 | 10.5 | 12.6 | 4.7 | 8.7 | 10.5 | 5.1 |
| weeks | (N = 86) | | | | | | | | | |
| Continued | 25 mg b.i.d. | 0.0 | 11.1 | 7.9 | 6.9 | 5.5 | 8.8 | 6.0 | 4.3 | 6.9 |
| b.i.d. | (N = 69) | | | | | | | | | |
| dosage | 50 mg b.i.d. | 0.0 | 24.4 | 17.9 | 10.4 | 16.4 | 12.7 | 14.6 | 12.2 | 8.2 |
| regimen | (N = 85) | | | | | | | | | |
| over 24 | 100 mg b.i.d. | 0.0 | 28.3 | 22.6 | 17.8 | 13.0 | 9.5 | 10.3 | 12.5 | 6.8 |
| weeks | (N = 84) | | | | | | | | | |
| Increased | from 50 mg | 0.0 | 8.1 | 9.0 | 5.0 | 13.2 | 13.5 | 11.9 | 11.6 | 2.9 |
| dosage | q.d. to 100 mg | | | | | | | | | |
| regimen | q.d. (N = 19) | | | | | | | | | |
| at 12 | from 25 mg | 0.0 | 4.6 | 3.7 | −5.6 | −0.5 | 3.2 | 13.7 | 10.1 | −2.1 |
| weeks | b.i.d. to 50 mg | | | | | | | | | |
| | b.i.d. (N = 17) | | | | | | | | | |

TABLE LV
Study 2 - Hemoglobin Mean CFB (g/L) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.0 | 0.0 | 0.0 | 0.0 |
| 1 | −1.1 | −0.4 | −1.7 | 0.4 |
| 2 | −1.1 | −2.5 | −2.0 | 0.2 |
| 4 | −0.6 | −0.2 | 0.0 | 1.7 |
| 8 | −1.9 | 0.9 | 1.8 | 2.5 |
| 12 | 0.2 | 1.8 | 3.1 | 3.9 |

TABLE LVI
Study 2 - Hemoglobin Mean CFB (g/L) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching | 0.0 | −1.1 | −1.1 | −0.6 | −1.9 | 0.2 | 1.0 | 3.0 | 3.4 |
| to 100 mg q.d. | | | | | | | | | |
| (N = 72) | | | | | | | | | |
| 50 mg q.d. | 0.0 | −0.8 | −3.0 | −0.4 | 1.1 | 2.6 | 1.8 | 0.6 | 2.0 |
| responders | | | | | | | | | |
| (N = 57) | | | | | | | | | |
| 50 mg q.d. non | 0.0 | 1.1 | −0.5 | 0.6 | 0.1 | −1.8 | 0.7 | 1.7 | 6.9 |
| responders | | | | | | | | | |
| switching to | | | | | | | | | |
| 100 mg q.d. | | | | | | | | | |
| (N = 15) | | | | | | | | | |
| 100 mg q.d. | 0.0 | −1.7 | −2.0 | 0.0 | 1.8 | 3.1 | 2.7 | 3.7 | 2.8 |
| (N = 70) | | | | | | | | | |
| 200 mg q.d. | 0.0 | 0.4 | 0.2 | 1.7 | 2.5 | 3.9 | 4.0 | 3.1 | 2.4 |
| (N = 69) | | | | | | | | | |

TABLE LVII
Study 2 - Hemoglobin Mean percentage
CFB (%) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | −0.74 | −0.21 | −1.20 | 0.55 |
| 2 | −0.67 | −1.80 | −1.34 | 0.34 |
| 4 | −0.29 | 0.04 | 0.21 | 1.55 |
| 8 | −1.33 | 0.90 | 1.60 | 2.23 |
| 12 | 0.33 | 1.60 | 2.83 | 3.42 |

TABLE LVIII
Study 2 - Hemoglobin Mean percentage CFB (%) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | −0.74 | −0.67 | −0.29 | −1.33 | 0.33 | 0.98 | 2.65 | 2.88 |
| 100 mg q.d. (N = 72) | | | | | | | | | |
| 50 mg q.d. responders | 0.00 | −0.50 | −2.17 | −0.08 | 1.18 | 2.35 | 1.78 | 0.76 | 1.79 |
| (N = 57) | | | | | | | | | |
| 50 mg q.d. non | 0.00 | 0.85 | −0.39 | 0.48 | −0.09 | −1.47 | 0.67 | 1.35 | 5.48 |
| responders switching to | | | | | | | | | |
| 100 mg q.d. (N = 15) | | | | | | | | | |
| 100 mg q.d. (N = 70) | 0.00 | −1.20 | −1.34 | 0.21 | 1.60 | 2.83 | 2.54 | 3.27 | 2.64 |
| 200 mg q.d. (N = 69) | 0.00 | 0.55 | 0.34 | 1.55 | 2.23 | 3.42 | 3.58 | 2.95 | 2.19 |

TABLE LIX
Study 2 - Neutrophils Mean CFB (giga/L) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | −0.28 | −0.22 | −0.23 | −0.39 |
| 2 | 0.35 | −0.10 | −0.73 | −0.67 |
| 4 | 0.13 | −0.57 | −0.63 | −1.15 |
| 8 | 0.21 | −0.63 | −1.07 | −1.14 |
| 12 | −0.22 | −0.30 | −1.12 | −1.25 |

TABLE LX
Study 2 - Neutrophils Mean percentage CFB (%) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | 1.38 | 4.30 | 0.16 | −1.22 |
| 2 | 9.12 | 3.82 | −8.24 | −2.38 |
| 4 | 9.88 | −1.97 | −4.01 | −11.41 |
| 8 | 10.55 | −1.56 | −11.07 | −7.63 |
| 12 | −4.19 | −5.40 | −16.57 | −21.33 |

TABLE LXI
Study 2 - Neutrophils Mean CFB (giga/L) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.0 | −0.28 | 0.35 | 0.12 | 0.21 | −0.22 | −1.20 | −1.17 | −0.66 |
| 100 mg q.d. (N = 72) | | | | | | | | | |
| 50 mg q.d. responders | 0.0 | −0.26 | −0.10 | −0.64 | −0.44 | −0.16 | −0.30 | −0.68 | −0.56 |
| (N = 57) | | | | | | | | | |
| 50 mg q.d. non | 0.0 | −0.07 | −0.11 | −0.29 | −1.34 | −0.87 | −1.22 | −1.57 | −2.34 |
| responders switching to | | | | | | | | | |
| 100 mg q.d. (N = 15) | | | | | | | | | |
| 100 mg q.d. (N = 70) | 0.0 | −0.23 | −0.73 | −0.63 | −1.07 | −1.12 | −1.19 | −1.28 | −1.04 |
| 200 mg q.d. (N = 69) | 0.0 | −0.39 | −0.67 | −1.15 | −1.14 | −1.25 | −0.99 | −1.02 | −1.43 |

TABLE LXII
Study 2 - Neutrophils Mean percentage CFB (%) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | 1.38 | 9.12 | 9.88 | 10.55 | 3.25 | −11.98 | −11.41 | −4.82 |
| 100 mg q.d. (N = 72) | | | | | | | | | |
| 50 mg q.d. responders | 0.00 | 5.63 | 3.97 | −2.31 | 2.24 | 7.79 | 6.50 | −1.27 | 3.33 |
| (N = 57) | | | | | | | | | |
| 50 mg q.d. non | 0.00 | −0.29 | 3.18 | −0.55 | −15.95 | −8.69 | −13.84 | −17.82 | −30.49 |
| responders switching to | | | | | | | | | |
| 100 mg q.d. (N = 15) | | | | | | | | | |
| 100 mg q.d. (N = 70) | 0.00 | 0.16 | −8.24 | −4.01 | −11.07 | −13.29 | −14.09 | −15.66 | −11.22 |
| 200 mg q.d. (N = 69) | 0.00 | −1.22 | −2.38 | −11.41 | −7.63 | −7.90 | −7.96 | −5.38 | −12.63 |

TABLE LXIII
Study 2 - Platelets Mean CFB (giga/L) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.0 | 0.0 | 0.0 | 0.0 |
| 1 | 3.6 | 9.1 | 2.6 | 2.4 |
| 2 | 13.2 | 5.8 | −16.7 | −12.6 |
| 4 | 11.3 | −13.2 | −13.5 | −27.1 |
| 8 | 16.0 | −8.4 | −29.7 | −19.4 |
| 12 | 8.2 | −11.1 | −30.4 | −27.9 |

TABLE LXIV
Study 2 - Platelets Mean percentage CFB (%) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | 1.97 | 4.86 | 2.20 | 2.43 |
| 2 | 4.87 | 4.74 | −2.43 | −2.29 |
| 4 | 4.89 | −0.44 | −2.70 | −6.66 |
| 8 | 6.41 | 0.90 | −7.68 | −4.88 |
| 12 | 3.59 | 0.14 | −7.08 | −7.23 |

TABLE LXV
Study 2 - Platelets Mean CFB (giga/L) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | 3.63 | 13.24 | 11.29 | 16.04 | 8.15 | −22.63 | −31.03 | −31.80 |
| 100 mg q.d. (N = 72) | | | | | | | | | |
| 50 mg q.d. responders | 0.00 | 5.71 | 4.81 | −11.47 | −7.81 | −10.28 | −9.94 | −13.50 | −26.13 |
| (N = 57) | | | | | | | | | |
| 50 mg q.d. non | 0.00 | 21.33 | 9.64 | −20.00 | −10.33 | −14.31 | −10.93 | −15.67 | −28.62 |
| responders switching to | | | | | | | | | |
| 100 mg q.d. (N = 15) | | | | | | | | | |
| 100 mg q.d. (N = 70) | 0.00 | 2.57 | −16.69 | −13.51 | −29.68 | −30.43 | −34.29 | −33.78 | −44.70 |
| 200 mg q.d. (N = 69) | 0.00 | 2.40 | −12.60 | −27.06 | −19.35 | −27.86 | −35.31 | −26.67 | −22.29 |

TABLE LXVI
Study 2 - Platelets Mean percentage CFB (%) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | 1.97 | 4.87 | 4.89 | 6.41 | 3.59 | −5.92 | −7.25 | −8.40 |
| 100 mg q.d. (N = 72) | | | | | | | | | |
| 50 mg q.d. responders | 0.00 | 4.19 | 5.23 | 1.22 | 1.76 | 1.22 | 0.77 | −0.91 | −5.56 |
| (N = 57) | | | | | | | | | |
| 50 mg q.d. non | 0.00 | 7.30 | 2.86 | −6.94 | −2.17 | −4.27 | −3.16 | −4.72 | −8.96 |
| responders switching to | | | | | | | | | |
| 100 mg q.d. (N = 15) | | | | | | | | | |
| 100 mg q.d. (N = 70) | 0.00 | 2.20 | −2.43 | −2.70 | −7.68 | −7.08 | −8.78 | −8.24 | −11.92 |
| 200 mg q.d. (N = 69) | 0.00 | 2.43 | −2.29 | −6.66 | −4.88 | −7.23 | −9.34 | −6.09 | −5.66 |

TABLE LXVII
Study 2 - Lymphocytes Mean CFB (giga/L) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | −0.02 | 0.11 | 0.30 | 0.18 |
| 2 | −0.07 | 0.2 | 0.15 | 0.13 |
| 4 | 0.15 | 0.01 | 0.15 | −0.04 |
| 8 | 0.13 | 0.05 | 0.07 | 0.04 |
| 12 | 0.20 | −0.05 | 0.21 | 0.02 |

TABLE LXVIII
Study 2 - Lymphocytes Mean percentage
CFB (%) - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| 0 | 0.00 | 0.00 | 0.00 | 0.00 |
| 1 | 1.36 | 7.55 | 21.30 | 17.99 |
| 2 | −0.49 | 4.70 | 16.36 | 13.26 |
| 4 | 11.84 | 6.02 | 13.84 | 3.82 |
| 8 | 10.37 | 9.66 | 9.91 | 10.13 |
| 12 | 13.70 | 3.88 | 20.06 | 11.79 |

TABLE LXIX
Study 2 - Lymphocytes Mean CFB (giga/L) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | −0.02 | −0.07 | 0.15 | 0.13 | 0.20 | 0.35 | 0.21 | 0.10 |
| 100 mg q.d. (N = 72) | | | | | | | | | |
| 50 mg q.d. responders | 0.00 | 0.07 | 0.01 | −0.07 | −0.06 | −0.11 | 0.01 | −0.05 | −0.10 |
| (N = 57) | | | | | | | | | |
| 50 mg q.d. non | 0.00 | 0.26 | 0.08 | 0.35 | 0.49 | 0.17 | 0.49 | 0.44 | 0.36 |
| responders switching to | | | | | | | | | |
| 100 mg q.d. (N = 15) | | | | | | | | | |
| 100 mg q.d. (N = 70) | 0.00 | 0.30 | 0.15 | 0.15 | 0.07 | 0.21 | 0.31 | 0.16 | 0.10 |
| 200 mg q.d. (N = 69) | 0.00 | 0.18 | 0.13 | −0.04 | 0.04 | 0.03 | −0.04 | −0.04 | 0.09 |

TABLE LXX
Study 2 - Lymphocytes Mean percentage CFB (%) - 24 weeks results

| | Weeks | | | | | | | | |
|---|---|---|---|---|---|---|---|---|---|
| | 0 | 1 | 2 | 4 | 8 | 12 | 16 | 20 | 24 |
| Placebo switching to | 0.00 | 1.36 | −0.49 | 11.84 | 10.37 | 13.70 | 22.23 | 16.32 | 12.38 |
| 100 mg q.d. (N = 72) | | | | | | | | | |
| 50 mg q.d. responders | 0.00 | 5.16 | 3.04 | 0.80 | 0.88 | 0.32 | 5.83 | 2.62 | 0.20 |
| (N = 57) | | | | | | | | | |
| 50 mg q.d. non | 0.00 | 15.85 | 11.48 | 27.68 | 42.86 | 18.11 | 34.64 | 32.07 | 29.58 |
| responders switching to | | | | | | | | | |
| 100 mg q.d. (N = 15) | | | | | | | | | |
| 100 mg q.d. (N = 70) | 0.00 | 21.30 | 16.36 | 13.84 | 9.91 | 20.06 | 24.07 | 15.66 | 11.70 |
| 200 mg q.d. (N = 69) | 0.00 | 17.99 | 13.26 | 3.82 | 10.13 | 11.79 | 6.58 | 8.53 | 10.80 |

TABLE LXXI
Study 3 - Hemoglobin Mean CFB (g/L) - 10 weeks results

| Weeks | Placebo (N = 44) | 200 mg q.d. (N = 130) |
|---|---|---|
| 0 | 0.0 | 0.0 |
| 2 | 1.5 | −0.9 |
| 4 | −0.2 | −1.4 |
| 6 | 0.0 | −0.6 |
| 10 | 2.6 | 2.6 |

TABLE LXXII
Study 3 - Hemoglobin Mean CFB (%) - 10 weeks results

| Weeks | Placebo (N = 44) | 200 mg q.d. (N = 130) |
|---|---|---|
| 0 | 0.00 | 0.00 |
| 2 | 1.7 | −0.5 |
| 4 | 0.3 | −0.7 |
| 6 | 0.3 | −0.2 |
| 10 | 2.8 | 2.4 |

TABLE LXXIII
Study 3 - Neutrophils Mean CFB (giga/L) - 10 weeks results

| Weeks | Placebo (N = 44) | 200 mg q.d. (N = 130) |
|---|---|---|
| 0 | 0.0 | 0.0 |
| 2 | 0.439 | −0.278 |
| 4 | 0.244 | −0.590 |
| 6 | 0.696 | −0.403 |
| 10 | 0.093 | −0.223 |

TABLE LXXIV
Study 3 - Neutrophils Mean percentage CFB (%) - 10 weeks results

| Weeks | Placebo (N = 44) | 200 mg q.d. (N = 130) |
|---|---|---|
| 0 | 0.00 | 0.00 |
| 2 | 12.462 | 0.048 |
| 4 | 12.997 | −3.522 |
| 6 | 25.753 | −1.039 |
| 10 | 10.064 | 3.058 |

TABLE LXXV
Study 3 - Platelets Mean CFB (giga/L) - 10 weeks results

| Weeks | Placebo (N = 44) | 200 mg q.d. (N = 130) |
|---|---|---|
| 0 | 0.0 | 0.0 |
| 2 | 3.3 | −6.5 |
| 4 | −17.6 | −16.8 |
| 6 | −2.6 | −10.5 |
| 10 | 1.1 | −19.3 |

TABLE LXXVI
Study 3 - Platelets Mean percentage CFB (%) - 10 weeks results

| Weeks | Placebo (N = 44) | 200 mg q.d. (N = 130) |
|---|---|---|
| 0 | 0.00 | 0.00 |
| 2 | 3.5 | −0.6 |
| 4 | −2.8 | −3.6 |
| 6 | 1.2 | −1.6 |
| 10 | 1.2 | −3.2 |

TABLE LXXVII
Study 3 - Lymphocytes Mean CFB (giga/L) - 10 weeks results

| Weeks | Placebo (N = 44) | 200 mg q.d. (N = 130) |
|---|---|---|
| 0 | 0.0 | 0.0 |
| 2 | 0.080 | 0.220 |
| 4 | 0.147 | 0.217 |
| 6 | −0.143 | 0.247 |
| 10 | 0.090 | 0.010 |

TABLE LXXVIII
Study 3 - Lymphocytes Mean percentage
CFB (%) - 10 weeks results

| Weeks | Placebo (N = 44) | 200 mg q.d. (N = 130) |
|---|---|---|
| 0 | 0.00 | 0.00 |
| 2 | 14.922 | 16.690 |
| 4 | 20.927 | 16.358 |
| 6 | 2.700 | 19.678 |
| 10 | 16.061 | 11.719 |

TABLE LXXIX
Study 3 - Hemoglobin Mean CFB (g/L) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 0.91 | 0.18 | −1.25 | 4.47 | 4.13 | 1.77 | 3.17 |
| | placebo (N = 22) | | | | | | | | |
| | To 100 mg q.d. | 0.0 | 2.00 | −0.41 | 0.91 | 1.41 | −1.05 | 2.36 | 1.24 |
| | (N = 22) | | | | | | | | |
| 200 mg | Continued | 0.0 | −1.24 | −2.36 | −0.96 | 3.36 | −0.36 | 0.14 | −0.26 |
| q.d. | 200 mg q.d. | | | | | | | | |
| | (N = 77) | | | | | | | | |
| | To 100 mg q.d. | 0.0 | −1.20 | −0.93 | −0.53 | 2.13 | 0.52 | −1.89 | 1.08 |
| | (N = 30) | | | | | | | | |
| | To placebo | 0.0 | 0.35 | 1.00 | 0.48 | 0.68 | 0.74 | 2.38 | 1.67 |
| | (N = 23) | | | | | | | | |

TABLE LXXX
Study 3 - Hemoglobin Mean CFB (%) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 1.27 | 0.55 | −0.68 | 4.23 | 3.73 | 1.88 | 3.40 |
| | placebo (N = 22) | | | | | | | | |
| | To 100 mg q.d. | 0.0 | 2.09 | 0.08 | 1.07 | 1.90 | −0.55 | 2.15 | 1.43 |
| | (N = 22) | | | | | | | | |
| 200 mg | Continued | 0.0 | −0.59 | −1.35 | −0.43 | 3.17 | 0.45 | 0.68 | 0.38 |
| q.d. | 200 mg q.d. | | | | | | | | |
| | (N = 77) | | | | | | | | |
| | To 100 mg q.d. | 0.0 | −0.83 | −0.37 | 0.02 | 2.01 | 0.77 | −1.19 | 1.33 |
| | (N = 30) | | | | | | | | |
| | To placebo | 0.0 | 0.22 | 0.71 | 0.31 | 0.58 | 0.54 | 1.96 | 1.24 |
| | (N = 23) | | | | | | | | |

TABLE LXXXI
Study 3 - Neutrophils Mean CFB (giga/L) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 1.04 | 0.60 | 0.90 | 0.18 | 0.89 | 0.89 | 0.62 |
| | placebo (N = 22) | | | | | | | | |
| | To 100 mg q.d. | 0.0 | −0.17 | −0.03 | 0.55 | 0.03 | 0.02 | 0.50 | 0.39 |
| | (N = 22) | | | | | | | | |
| 200 mg | Continued | 0.0 | 0.05 | −0.23 | −0.18 | −0.01 | −0.19 | −0.40 | −0.16 |
| q.d. | 200 mg q.d. | | | | | | | | |
| | (N = 77) | | | | | | | | |
| | To 100 mg q.d. | 0.0 | −0.90 | −1.22 | −0.54 | −0.33 | −1.18 | −0.32 | −0.96 |
| | (N = 30) | | | | | | | | |
| | To placebo | 0.0 | −0.53 | −0.97 | −0.88 | −0.67 | −0.14 | 0.16 | 0.19 |
| | (N = 23) | | | | | | | | |

TABLE LXXXII
Study 3 - Neutrophils Mean percentage CFB (%) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 19.87 | 20.58 | 35.04 | 10.46 | 20.49 | 22.59 | 16.71 |
| | placebo (N = 22) | | | | | | | | |
| | To 100 mg q.d. | 0.0 | 5.05 | 7.14 | 19.00 | 9.79 | 12.63 | 19.69 | 24.21 |
| | (N = 22) | | | | | | | | |
| 200 mg | Continued | 0.0 | 3.81 | 1.74 | 2.75 | 6.71 | 2.94 | 1.55 | 6.00 |
| q.d. | 200 mg q.d. | | | | | | | | |
| | (N = 77) | | | | | | | | |
| | To 100 mg q.d. | 0.0 | −5.34 | −12.20 | −2.48 | 3.61 | −10.54 | 0.82 | −9.59 |
| | (N = 30) | | | | | | | | |
| | To placebo | 0.0 | −5.36 | −9.66 | −10.53 | −7.51 | 1.36 | 6.34 | 4.90 |
| | (N = 23) | | | | | | | | |

TABLE LXXXIII
Study 3 - Platelets Mean CFB (giga/L) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 2.86 | −42.06 | −24.63 | −11.20 | −9.20 | −18.77 | −6.08 |
| | placebo (N = 22) | | | | | | | | |
| | To 100 mg q.d. | 0.0 | 3.73 | 1.32 | 13.36 | 9.50 | 18.73 | 16.00 | 18.24 |
| | (N = 22) | | | | | | | | |
| 200 mg | Continued | 0.0 | −8.96 | −17.36 | −11.03 | −14.31 | −9.55 | −13.40 | −13.43 |
| q.d. | 200 mg q.d. | | | | | | | | |
| | (N = 77) | | | | | | | | |
| | To 100 mg q.d. | 0.0 | 2.43 | −22.48 | −17.13 | −19.50 | −16.21 | 2.25 | −3.23 |
| | (N = 30) | | | | | | | | |
| | To placebo | 0.0 | −10.04 | −7.91 | −0.04 | −28.82 | −16.91 | −17.10 | 3.95 |
| | (N = 23) | | | | | | | | |

TABLE LXXXIV
Study 3 - Platelets Mean percentage CFB (%) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 4.35 | −7.51 | −3.25 | −0.78 | 2.12 | −1.34 | 0.07 |
| | placebo (N = 22) | | | | | | | | |
| | To 100 mg q.d. | 0.0 | 2.56 | 0.84 | 4.48 | 2.62 | 4.58 | 3.99 | 5.36 |
| | (N = 22) | | | | | | | | |
| 200 mg | Continued | 0.0 | −2.07 | −4.17 | −2.03 | −1.69 | −0.34 | −1.47 | −1.26 |
| q.d. | 200 mg q.d. | | | | | | | | |
| | (N = 77) | | | | | | | | |
| | To 100 mg q.d. | 0.0 | 1.71 | −5.62 | −3.89 | −4.48 | −3.42 | 2.00 | 0.37 |
| | (N = 30) | | | | | | | | |
| | To placebo | 0.0 | 1.12 | 0.54 | 2.92 | −4.41 | −0.24 | −0.50 | 4.45 |
| | (N = 23) | | | | | | | | |

TABLE LXXXV
Study 3 - Lymphocytes Mean CFB (giga/L) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 0.65 | −0.63 | −0.62 | 0.54 | 0.62 | −3.23 | −1.55 |
| | placebo (N = 22) | | | | | | | | |
| | To 100 mg q.d. | 0.0 | −0.19 | 1.41 | −3.31 | −0.07 | −0.21 | −1.96 | −1.60 |
| | (N = 22) | | | | | | | | |
| 200 mg | Continued | 0.0 | 1.89 | 2.62 | 2.61 | 1.06 | 0.89 | 2.98 | 1.18 |
| q.d. | 200 mg q.d. | | | | | | | | |
| | (N = 77) | | | | | | | | |
| | To 100 mg q.d. | 0.0 | 4.19 | 4.53 | 2.50 | 1.24 | 2.11 | 1.80 | 2.33 |
| | (N = 30) | | | | | | | | |
| | To placebo | 0.0 | 5.25 | 4.26 | 3.81 | 1.80 | −1.47 | −0.68 | −1.09 |
| | (N = 23) | | | | | | | | |

TABLE LXXXVI
Study 3 - Lymphocytes Mean percentage CFB (%) - 20 weeks results

| Week 0 to | Week 10 to | | | | | Week | Week | Week | Week |
|---|---|---|---|---|---|---|---|---|---|
| week 10 | week 20 | Week 0 | Week 2 | Week 4 | Week 6 | 10 | 12 | 16 | 20 |
| Placebo | Continued | 0.0 | 7.24 | 2.33 | 5.59 | 9.24 | 6.57 | −13.76 | −1.41 |
| | placebo (N = 22) | | | | | | | | |
| | To 100 mg q.d. | 0.0 | 17.48 | 25.91 | −3.65 | 19.26 | 18.75 | 13.42 | 15.16 |
| | (N = 22) | | | | | | | | |
| 200 mg | Continued | 0.0 | 12.02 | 18.03 | 23.38 | 22.16 | 16.80 | 31.65 | 19.81 |
| q.d. | 200 mg q.d. | | | | | | | | |
| | (N = 77) | | | | | | | | |
| | To 100 mg q.d. | 0.0 | 28.44 | 35.03 | 20.92 | 14.51 | 21.71 | 17.78 | 25.68 |
| | (N = 30) | | | | | | | | |
| | To placebo | 0.0 | 31.26 | 26.02 | 27.85 | 16.28 | 2.15 | 3.04 | 0.68 |
| | (N = 23) | | | | | | | | |

### 3.5. DAS28(CRP)

The (DAS28(CRP)) is a system developed and validated by the European League Against Rheumatism (EULAR) to measure the progress and improvement of rheumatoid arthritis and has been extensively validated (Wells et al., 2008). The DAS28(CRP) scoring includes a 28 tender and swollen joint count, CRP measurement from blood analysis, and a general health assessment on a visual analog scale (Fransen et al., 2003).

DAS28(CRP) values range from 2.0 to 10.0, and more particularly reflect the following status:
Remission: DAS28(CRP) ≤2.6
Low Disease activity: 2.6 <DAS28(CRP) ≤3.2
Moderate Disease Activity: 3.2 <DAS28(CRP) ≤5.1
High Disease Activity: DAS28(CRP) >5.1

In practice, the DAS28(CRP) measurement involves the evaluation 28 different joints including in the measurement (proximal interphalangeal joints (10 joints), metacarpophalangeal joints (10), wrists (2), elbows (2), shoulders (2), and knees (2)). When looking at these joints, both the number of joints with tenderness upon touching and swelling are counted.

Secondly, C-reactive protein level is measured.

Finally, the patient makes a subjective assessment of disease activity during the preceding 7 days on a scale between 0 and 100, where 0 is “no activity” and 100 is “highest activity possible”.

The DAS28(CRP) score is then calculated as follows:

Firstly, the patient is asked to make a vertical mark on a 100 mm Visual Analog Scale (VAS) corresponding to their general health or global disease activity on a touch screen to measure the VAS value, which is used in the formula below.

Secondly, a swollen and tender joint examination is then performed on the patient. The swollen and tender joints are recorded. From this examination are obtained the total amount of swollen joints (SJC) and the total amount of tender joints (TJC), which are used in the formula below.

Thirdly, C-reactive protein (CRP) levels are measured.

Finally, the values obtained above (VAS, TJC28, SJC28, and CRP) are computed into the following Formula to obtain the DAS28(CRP) score.

DAS28(CRP)=0.56*\sqrt{TJC28}+0.28\sqrt{SJC28}+0.70 ln(CRP+1)+0.014*VAS

The improvement (decreases) in the DAS28(CRP) scores for each patient group at weeks 1, 2, 4, 8 and 12 is shown in Table LXXXVII and Table LXXXVIII below, together with the Hommel-corrected p-value for the pairwise comparisons of each group with placebo.

The number of subjects (N) provided in each groups corresponds to the number of subjects starting the study in each group, and the DAS28 (CRP) data reported below corresponds to the responding subjects continuing for the entire 24 weeks on their initial treatment course.

TABLE LXXXVII
Study 1 - DAS28(CRP) scores - 12 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| Week 1 | −0.56 | −0.65 | −1.01 | −1.17 | −0.68 | −0.84 | −1.33 |
| DAS28(CRP) mean | | (0.6456) | (0.0139) | (0.0003) | (0.6456) | (0.2320) | (<0.0001) |
| CFB (LOCF) | | | | | | | |
| (p-value vs placebo) | | | | | | | |
| Week 2 | −0.80 | −0.94 | −1.50 | −1.52 | −1.06 | −1.22 | −1.84 |
| DAS28(CRP) mean | | (0.4820) | (0.0002) | (0.0002) | (0.3074) | (0.0535) | (<0.0001) |
| CFB (LOCF) | | | | | | | |
| (p-value vs placebo) | | | | | | | |
| Week 4 | −0.97 | −1.28 | −1.88 | −1.92 | −1.45 | −1.65 | −2.28 |
| DAS28(CRP) mean | | (0.1226) | (<0.0001) | (<0.0001) | (0.0275) | (0.0012) | (<0.0001) |
| CFB (LOCF) | | | | | | | |
| (p-value vs placebo) | | | | | | | |
| Week 8 | −1.15 | −1.66 | −2.12 | −2.30 | −1.82 | −1.93 | −2.72 |
| DAS28(CRP) mean | | (0.0129) | (<0.0001) | (<0.0001) | (0.0018) | (0.0003) | (<0.0001) |
| CFB (LOCF) | | | | | | | |
| (p-value vs placebo) | | | | | | | |
| Week 12 | −1.20 | −1.75 | −2.21 | −2.49 | −1.88 | −2.10 | −2.84 |
| DAS28(CRP) mean | | (0.0105) | (<0.0001) | (<0.0001) | (0.0026) | (<0.0001) | (<0.0001) |
| CFB (LOCF) | | | | | | | |
| (p-value vs placebo) | | | | | | | |
| % patients reaching | 7% | 12% | 13% | 15% | 13% | 11% | 14% |
| low disease activity at | | | | | | | |
| week 12 (LOCF) | | | | | | | |
| % patients reaching | 7% | 12% | 21% | 22% | 15% | 18% | 36% |
| remission at week 12 | | | | | | | |
| (LOCF) | | | | | | | |
| % patients reaching | 14% | 24% | 34% | 37% | 28% | 29% | 50% |
| low disease activity | | | | | | | |
| and/or remission at | | | | | | | |
| week 12 (LOCF) | | | | | | | |

TABLE LXXXVIII
Study 1 - DAS28(CRP) scores - 24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| Week 1 | −0.57 | −0.65 | −1.00 | −1.17 | −0.68 | −0.84 | −1.33 |
| DAS28(CRP) mean | | (0.6483) | (0.0179) | (0.0003) | (0.6483) | (0.2424) | (<0.0001) |
| CFB (LOCF) | | | | | | | |
| (p-value vs placebo) | | | | | | | |
| Week 2 | −0.80 | −0.94 | −1.51 | −1.52 | −1.06 | −1.22 | −1.84 |
| DAS28(CRP) mean | | (0.4843) | (0.0002) | (0.0002) | (0.3098) | (0.0600) | (<0.0001) |
| CFB (LOCF) | | | | | | | |
| (p-value vs placebo) | | | | | | | |
| Week 4 | −0.97 | −1.28 | −1.89 | −1.92 | −1.45 | −1.63 | −2.24 |
| DAS28(CRP) mean | | (0.1236) | (<0.0001) | (<0.0001) | (0.0280) | (0.0018) | (<0.0001) |
| CFB (LOCF) | | | | | | | |
| (p-value vs placebo) | | | | | | | |
| Week 8 | −1.15 | −1.66 | −2.13 | −2.31 | −1.82 | −1.93 | −2.72 |
| DAS28(CRP) mean | | (0.0131) | (<0.0001) | (<0.0001) | (0.0018) | (0.0003) | (<0.0001) |
| CFB (LOCF) | | | | | | | |
| (p-value vs placebo) | | | | | | | |
| Week 12 | −1.19 | −1.75 | −2.23 | −2.47 | −1.88 | −2.10 | −2.84 |
| DAS28(CRP) mean | | (0.0092) | (<0.0001) | (<0.0001) | (<0.0001) | (<0.0001) | (<0.0001) |
| CFB (LOCF) | | | | | | | |
| (p-value vs placebo) | | | | | | | |
| Week 16 | −1.35 | −1.91 | −2.51 | −2.70 | −2.02 | −2.37 | −3.06 |
| DAS28(CRP) mean | | (0.0117) | (<0.0001) | (<0.0001) | (<0.0001) | (<0.0001) | (<0.0001) |
| CFB (LOCF) | | | | | | | |
| (p-value vs placebo) | | | | | | | |
| Week 20 | −1.26 | −1.99 | −2.65 | −2.86 | −2.07 | −2.40 | −3.02 |
| DAS28(CRP) mean | | (0.0009) | (<0.0001) | (<0.0001) | (<0.0001) | (<0.0001) | (<0.0001) |
| CFB (LOCF) | | | | | | | |
| (p-value vs placebo) | | | | | | | |
| Week 24 | −1.18 | −1.98 | −2.70 | −2.80 | −2.19 | −2.40 | −3.23 |
| DAS28(CRP) mean | | (0.0004) | (<0.0001) | (<0.0001) | (<0.0001) | (<0.0001) | (<0.0001) |
| CFB (LOCF) | | | | | | | |
| (p-value vs placebo) | | | | | | | |
| % patients reaching | 9 | 12 | 14 | 26 | 16 | 14 | 24 |
| low disease activity at | | | | | | | |
| week 24 (LOCF) | | | | | | | |
| % patients reaching | 9 | 21 | 36 | 26 | 23 | 24 | 40 |
| remission at week 24 | | | | | | | |
| (LOCF) | | | | | | | |
| % patients reaching | 19 | 33 | 51 | 51 | 40 | 38 | 64 |
| low disease activity | | | | | | | |
| and/or remission at | | | | | | | |
| week 24 (LOCF) | | | | | | | |

TABLE LXXXIX
Study 2 - DAS28(CRP) scores - 12 weeks results

| | | 50 mg | 100 mg | 200 mg |
|---|---|---|---|---|
| | Placebo | q.d. | q.d. | q.d. |
| | (N = 72) | (N = 72) | (N = 70) | (N = 69) |
| Week 1 | −0.51 | −0.70 | −0.88 | −1.15 |
| DAS28(CRP) mean | | (0.0992) | (0.0269) | (<0.0001) |
| CFB (LOCF) | | | | |
| (p-value vs placebo) | | | | |
| Week 2 | −0.74 | −0.97 | −1.04 | −1.54 |
| DAS28(CRP) mean | | (0.0925) | (0.0925) | (<0.0001) |
| CFB (LOCF) | | | | |
| (p-value vs placebo) | | | | |
| Week 4 | −0.84 | −1.37 | −1.50 | −1.87 |
| DAS28(CRP) mean | | (0.0021) | (0.0010) | (<0.0001) |
| CFB (LOCF) | | | | |
| (p-value vs placebo) | | | | |
| Week 8 | −0.94 | −1.65 | −1.91 | −2.24 |
| DAS28(CRP) mean | | (0.0001) | (<0.0001) | (<0.0001) |
| CFB (LOCF) | | | | |
| (p-value vs placebo) | | | | |
| Week 12 | −0.99 | −1.69 | −2.04 | −2.33 |
| DAS28(CRP) mean | | (0.0006) | (<0.0001) | (<0.0001) |
| CFB (LOCF) | | | | |
| (p-value vs placebo) | | | | |
| % patients reaching | 7% | 13% | 13% | 28% |
| low disease activity | | | | |
| at week 12 (LOCF) | | | | |
| % patients reaching | 7% | 11% | 14% | 17% |
| remission at week | | | | |
| 12 (LOCF) | | | | |
| % patients reaching | 14% | 24% | 28% | 45% |
| low disease activity | | | | |
| and/or remission at | | | | |
| week 12 (LOCF) | | | | |

TABLE XC
Study 2 - DAS28(CRP) scores - 24 weeks results

| | | 50 mg | 100 mg | 200 mg |
|---|---|---|---|---|
| | Placebo | q.d. | q.d. | q.d. |
| | (N = 72) | (N = 72) | (N = 70) | (N = 69) |
| Week 1 | −0.51 | −0.75 | −0.87 | −1.16 |
| DAS28(CRP) mean | | (0.0585) | (0.0247) | (<0.0001) |
| CFB (LOCF) | | | | |
| (p-value vs placebo) | | | | |
| Week 2 | −0.74 | −1.03 | −1.04 | −1.55 |
| DAS28(CRP) mean | | (0.0608) | (0.0608) | (<0.0001) |
| CFB (LOCF) | | | | |
| (p-value vs placebo) | | | | |
| Week 4 | −0.84 | −1.43 | −1.48 | −1.87 |
| DAS28(CRP) mean | | (0.0012) | (0.0012) | (<0.0001) |
| CFB (LOCF) | | | | |
| (p-value vs placebo) | | | | |
| Week 8 | −0.94 | −1.71 | −1.89 | −2.23 |
| DAS28(CRP) mean | | (<0.0001) | (<0.0001) | (<0.0001) |
| CFB (LOCF) | | | | |
| (p-value vs placebo) | | | | |
| Week 12 | −0.99 | −1.75 | −2.04 | −2.32 |
| DAS28(CRP) mean | | (0.0002) | (<0.0001) | (<0.0001) |
| CFB (LOCF) | | | | |
| (p-value vs placebo) | | | | |
| Week 16 | — | −1.88 | −2.39 | −2.53 |
| DAS28(CRP) mean | | | | |
| CFB (LOCF) | | | | |
| Week 20 | — | −1.83 | −2.48 | −2.55 |
| DAS28(CRP) mean | | | | |
| CFB (LOCF) | | | | |
| Week 24 | — | −1.95 | −2.61 | −2.62 |
| DAS28(CRP) mean | | | | |
| CFB (LOCF) | | | | |
| % patients reaching | — | 17 | 29 | 19 |
| low disease activity | | | | |
| at week 24 (LOCF) | | | | |
| % patients reaching | N/A | 19% | 21% | 28% |
| remission at week | | | | |
| 24 (LOCF) | | | | |
| % patients reaching | N/A | 36% | 50% | 46% |
| low disease activity | | | | |
| and/or remission at | | | | |
| week 24 (LOCF) | | | | |

### 3.6. CRP Analysis

### 3.6.1.1. Principle of the Assay

CRP determination is available at Quest Diagnostics, Clinical Trials, Quest House, 125-135 Staines Road, Hounslow, Middlesex, TW3 3JB, United Kingdom under Catalogue n#86140.

The determination of CRP is made using Immunoturbidimetric assay for the in vitro quantitative determination of CRP in human serum and plasma on Roche/Hitachi cobas c systems, wherein human CRP agglutinates with latex particles coated with monoclonal anti-CRP antibodies. The aggregates are determined turbidimetrically (Eda et al., 1998; Price et al., 1987).

### 3.6.1.2. Assay

Human serum and plasma samples CRP determination was done on a Roche/Hitachi Cobas c 301 c 501/502 system which automatically calculate the analyte concentration of each sample. Samples containing precipitates are centrifuged before performing the assay.

The machine parameters are listed in Table XCI and Table XCII below, and the results at 12 weeks v using either LOCF are shown in Table XCIII and below. The Hommel-corrected p-value for the pairwise comparisons of each group with placebo is shown.

The number of subjects (N) provided in each groups corresponds to the number of subjects starting the study in each group, and the CRP data reported below corresponds to the responding subjects continuing for the entire 24 weeks on their initial treatment course.

TABLE XCI
Cobas c 311 test parameters for CRP determination

| Assay type | | 2 Point End |
|---|---|---|
| Reaction time/Assay points | | 10/18-38 |
| Wavelength (sub/main) | | 800/570 nm |
| Reaction direction | | Increase |
| Units | | mg/L (nmol/L, mg/dL) |
| Reagent pipetting | | Diluent (H₂O) |
| Tris(hydroxymethyl)-aminomethane | | 150 μL |
| (TRIS) buffer with bovine serum | | |
| albumin; preservatives | | |
| Latex particles coated with anti- | | 48 μL + H₂O (24 μL) |
| CRP (mouse) in glycine buffer; | | |
| immunoglobulins (mouse); preservative | | |
| Sample volumes | Sample dilution | Diluent (NaCl) |
| Normal (2 μL) | — | — |
| Decreased (4 μL) | 25 μL | 75 μL |
| Increased (4 μL) | — | — |

TABLE XCII
Cobas c 501/502 test parameters for CRP determination

| Assay type | | 2 Point End |
|---|---|---|
| Reaction time/Assay points | | 10/13-29 |
| Wavelength (sub/main) | | 800/570 nm |
| Reaction direction | | Increase |
| Units | | mg/L (nmol/L, mg/dL) |
| Reagent pipetting | | Diluent (H₂O) |
| Tris(hydroxymethyl)-aminomethane | | 150 μL |
| (TRIS) buffer with bovine serum | | |
| albumin; preservatives | | |
| Latex particles coated with anti- | | 48 μL + H₂O (24 μL) |
| CRP (mouse) in glycine buffer; | | |
| immunoglobulins (mouse); preservative | | |
| Sample volumes | Sample dilution | Diluent (NaCl) |
| Normal (2 μL) | — | — |
| Decreased (4 μL) | 25 μL | 75 μL |
| Increased (4 μL) | — | — |

TABLE XCIII
Study 1 - CRP values - 12 weeks results

| | | q.d. groups | | | b.i.d. groups | | |
|---|---|---|---|---|---|---|---|
| | | 50 mg | | | 2 × | 2 × | 2 × |
| | Placebo | (N = | 100 mg | 200 mg | 25 mg | 50 mg | 100 mg |
| | (N = 86) | 82) | (N = 85) | (N = 86) | (N = 86) | (N = 85) | (N = 84) |
| Week 1 | 1.78 | −7.95 | −11.02 | −17.06 | −8.47 | −10.08 | −17.39 |
| CRP mean CFB mg/L | | (0.1495) | (0.0035) | (<0.0001) | (0.0980) | (0.0099) | (<0.0001) |
| (LOCF) | | | | | | | |
| (p-value vs placebo) | | | | | | | |
| Week 2 | 1.57 | −8.23 | −12.94 | −17.51 | −10.06 | −11.03 | −19.69 |
| CRP mean CFB mg/L | | (0.2029) | (<0.0001) | (<0.0001) | (0.0133) | (0.0009) | (<0.0001) |
| (LOCF) | | | | | | | |
| (p-value vs placebo) | | | | | | | |
| Week 4 | 2.26 | −10.18 | −13.18 | −17.94 | −7.72 | −13.28 | −20.64 |
| CRP mean CFB mg/L | | (0.0932) | (0.0001) | (<0.0001) | (0.1478) | (0.0001) | (<0.0001) |
| (LOCF) | | | | | | | |
| (p-value vs placebo) | | | | | | | |
| Week 8 | 1.47 | −12.02 | −12.48 | −17.22 | −10.94 | −13.25 | −21.32 |
| CRP mean CFB mg/L | | (0.0433) | (0.0052) | (0.0001) | (0.0433) | (0.0029) | (<0.0001) |
| (LOCF) | | | | | | | |
| (p-value vs placebo) | | | | | | | |
| Week 12 | 2.67 | −13.15 | −13.57 | −17.24 | −10.26 | −12.97 | −20.54 |
| CRP mean CFB mg/L | | (0.0103) | (0.0005) | (<0.0001) | (0.0273) | (0.0010) | (<0.0001) |
| (LOCF) | | | | | | | |
| (p-value vs placebo) | | | | | | | |

TABLE XCIV
Study 1 - CRP values - 24 weeks results

| | | q.d. groups | | | b.i.d. groups | | |
|---|---|---|---|---|---|---|---|
| | | 50 mg | | | 2 × | 2 × | 2 × |
| | Placebo | (N = | 100 mg | 200 mg | 25 mg | 50 mg | 100 mg |
| | (N = 86) | 82) | (N = 85) | (N = 86) | (N = 86) | (N = 85) | (N = 84) |
| Week 1 | 1.78 | −7.95 | −11.02 | −17.06 | −8.47 | −10.08 | −17.39 |
| CRP mean CFB mg/L | | (0.1495) | (0.0035) | (<0.0001) | (0.0980) | (0.0099) | (<0.0001) |
| (LOCF) | | | | | | | |
| (p-value vs placebo) | | | | | | | |
| Week 2 | 1.57 | −8.23 | −12.94 | −17.51 | −10.06 | −11.03 | −19.69 |
| CRP mean CFB mg/L | | (0.2029) | (<0.0001) | (<0.0001) | (0.0133) | (0.0009) | (<0.0001) |
| (LOCF) | | | | | | | |
| (p-value vs placebo) | | | | | | | |
| Week 4 | 2.26 | −10.18 | −13.18 | −17.94 | −7.72 | −13.28 | −20.64 |
| CRP mean CFB mg/L | | (0.0932) | (0.0001) | (<0.0001) | (0.1478) | (0.0001) | (<0.0001) |
| (LOCF) | | | | | | | |
| (p-value vs placebo) | | | | | | | |
| Week 8 | 1.47 | −12.02 | −12.48 | −17.22 | −10.94 | −13.25 | −21.32 |
| CRP mean CFB mg/L | | (0.0433) | (0.0052) | (0.0001) | (0.0433) | (0.0029) | (<0.0001) |
| (LOCF) | | | | | | | |
| (p-value vs placebo) | | | | | | | |
| Week 12 | 2.67 | −13.15 | −13.57 | −17.24 | −10.26 | −12.97 | −20.54 |
| CRP mean CFB mg/L | | (0.0103) | (0.0005) | (<0.0001) | (0.0273) | (0.0010) | (<0.0001) |
| (LOCF) | | | | | | | |
| (p-value vs placebo) | | | | | | | |
| Week 16 | 1.39 | −13.55 | −11.66 | −20.38 | −12.55 | −12.57 | −21.34 |
| CRP mean CFB mg/L | | (0.0046) | (0.0044) | (<0.0001) | (0.0046) | (0.0025) | (<0.0001 |
| (LOCF) | | | | | | | |
| (p-value vs placebo) | | | | | | | |
| Week 20 | 1.63 | −13.12 | −14.50 | −17.89 | −12.20 | −13.16 | −20.80 |
| CRP mean CFB mg/L | | (0.0094) | (<0.0001) | (<0.0001) | (0.0094) | (0.0007) | (<0.0001) |
| (LOCF) | | | | | | | |
| (p-value vs placebo) | | | | | | | |
| Week 24 | 2.00 | −15.22 | −14.89 | −15.57 | −11.68 | −11.96 | −20.82 |
| CRP mean CFB mg/L | | (0.0094) | (0.0008) | (0.0039) | (0.0280) | (0.0142) | (<0.0001) |
| (LOCF) | | | | | | | |
| (p-value vs placebo) | | | | | | | |

TABLE XCV
Study 2 - CRP values - 12 weeks results

| | Placebo | 50 mg q.d. | 100 mg q.d. | 200 mg q.d. |
|---|---|---|---|---|
| | (N = 72) | (N = 72) | (N = 70) | (N = 69) |
| Week 1 | −5.65 | −3.70 | −10.33 | −12.58 |
| CRP mean CFB | | (0.3187) | (0.0286) | (0.0020) |
| mg/L (LOCF) | | | | |
| (p-value vs | | | | |
| placebo) | | | | |
| Week 2 | −1.57 | −4.16 | −8.67 | −13.28 |
| CRP mean CFB | | (0.0613) | (0.0146) | (0.0002) |
| mg/L (LOCF) | | | | |
| (p-value vs | | | | |
| placebo) | | | | |
| Week 4 | −1.68 | −9.46 | −12.08 | −13.04 |
| CRP mean CFB | | (<0.0001) | (<0.0001) | (<0.0001) |
| mg/L (LOCF) | | | | |
| (p-value vs | | | | |
| placebo) | | | | |
| Week 8 | −3.66 | −11.16 | −14.17 | −13.86 |
| CRP mean CFB | | (<0.0001) | (<0.0001) | (<0.0001) |
| mg/L (LOCF) | | | | |
| (p-value vs | | | | |
| placebo) | | | | |
| Week 12 | −8.71 | −4.43 | −12.25 | −14.85 |
| CRP mean CFB | | (0.4200) | (0.0338) | (0.0021) |
| mg/L (LOCF) | | | | |
| (p-value vs | | | | |
| placebo) | | | | |

### 3.7. ACR Scores (ACR20/50/70)

The American College of Rheumatology (ACR) composite score, which evaluates clinical improvement relative to an initial assessment, is a commonly accepted efficacy end point for clinical trials in patients with rheumatoid arthritis. The ACR score correspond to a percentage improvement in pre-set parameters, and therefore measures the evolution of the disease in a patient. For example, ACR20 denotes at least 20% improvement in tender and swollen joint counts and in three of the following end points: global assessment by the patient, global assessment by the doctor, C reactive protein (CRP), patient's pain Visual Analogue Scale and Health Assessment Questionnaire Score. The number of swollen joints, ESR and CRP correlate with radiographic progression of joint damage, whereas tender joint counts, as well as global assessments by doctors and patients, and patients' reports of pain, are sensitive to change in clinical outcomes. Physical function, as assessed by the Health Assessment Questionnaire Score, is a potent predictor of the risk of disability in rheumatoid arthritis (Felson and American College of Rheumatology Committee to Reevaluate Improvement Criteria, 2007; Felson et al., 1993).

In particular, the list of parameters used to assess ACR score can be found in the Table XCVI below and the results of the ACR responses at week 12 via either NRI or LOCF are shown in the tables below. The Hommel-corrected p-value for the pairwise comparisons of each group with placebo is shown.

The number of subjects (N) provided in each groups corresponds to the number of subjects starting the study in each group, and the ACR data reported below corresponds to the responding subjects continuing for the entire 24 weeks on their initial treatment course.

TABLE XCVI
ACR activity points of measurement

| | Disease Activity | |
|---|---|---|
| Point | Measure | Method of Assessment |
| 1 | Tender joint | ACR tender joint count, an assessment of 68 joints. |
| | count | The 68 joints to be examined for tenderness are: temporomandibular (n = 2), |
| | | sternoclavicular (n = 2), acromioclavicular (n = 2), shoulder (n = 2), elbow (n = |
| | | 2), wrist (n = 2), metacarpophalageal (n = 10), interphalangeal of thumb (n = |
| | | 2), distal interphalangeal (n = 8), proximal interphalangeal (n = 8), hip (n = |
| | | 2), knee (n = 2), ankle mortise (n = 2), ankle tarsus (n = 2), |
| | | metatarsophalangeal (n = 10), interphalangeal of great toe (n = 2), and |
| | | proxima/distal interphalangeal of the toes (n = 8). |
| | | The joint count should be done by scoring several different aspects of |
| | | tenderness, as assessed by pressure and joint manipulation on physical |
| | | examination. The information on various types of tenderness should then be |
| | | collapsed into a single tender-versus-nontender dichotomy. |
| 2 | Swollen joint | ACR swollen joint count, an assessment of 66 joints. Joints are classified as |
| | count | either swollen or not swollen. |
| | | The 66 joints to be examined for swelling are the same as those examined for |
| | | tenderness, except the hip joints are not included. |
| 3 | Patient's | A horizontal visual analog scale (100 mm) assessment of the patient's current |
| | assessment of | level of pain. |
| | pain | |
| 4 | Patient's global | The patient's overall assessment of how the arthritis is doing. One acceptable |
| | assessment of | method for determining this is the question from the AIMS instrument: |
| | disease activity | “Considering all the ways your arthritis affects you, mark ‘X’ on the scale for |
| | | how well you are doing.” An anchored, horizontal, visual analog scale |
| | | (100 mm) should be provided. A Likert scale response is also acceptable. |
| 5 | Physician's | A horizontal visual analog scale (100 mm) measure of the physician's |
| | global | assessment of the patient's current disease activity. |
| | assessment of | |
| | disease activity | |
| 6 | Patient's | Any patient self-assessment instrument which has been validated, has |
| | assessment of | reliability, has been proven in RA trials to be sensitive to change, and which |
| | physical function | measures physical function in RA patients is acceptable. |
| | | In the present protocol we use HAQ-DI. |
| 7 | Acute-phase | A C-reactive protein level. |
| | reactant value | |

TABLE XCVII
Study 1 - ACR responses at week 1 - 12 weeks results

| | | q.d. groups | | | b.i.d. groups | | |
|---|---|---|---|---|---|---|---|
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 17.4% | 22.0% | 34.1% | 27.9% | 17.4% | 22.4% | 34.5% |
| (p-value vs placebo) | | (0.9378) | (0.0664) | (0.3908) | (0.9378) | (0.8540) | (0.0588) |
| ACR20 (LOCF) | 17.4% | 22.0% | 32.9% | 25.6% | 17.4% | 22.4% | 33.3% |
| (p-value vs placebo) | | (0.9399) | (0.1012) | (0.6350) | (0.9399) | (0.8525) | (0.0897) |
| ACR50 (NRI) | 1.2% | 2.4% | 5.9% | 4.7% | 3.5% | 7.1% | 7.1% |
| (p-value vs placebo) | | (0.5426) | (0.4047) | (0.5111) | (0.5426) | (0.3512) | (0.3373) |
| ACR50 (LOCF) | 1.2% | 2.4% | 5.9% | 4.7% | 2.3% | 7.1% | 7.1% |
| (p-value vs placebo) | | (0.5763) | (0.4036) | (0.5763) | (0.5763) | (0.3507) | (0.3362) |
| ACR70 (NRI) | 1.2% | 0.0% | 0.0% | 1.2% | 1.2% | 2.4% | 4.8% |
| (p-value vs placebo) | | (0.9962) | (0.9962) | (0.9962) | (0.9962) | (0.9962) | (0.9962) |
| ACR70 (LOCF) | 1.2% | 0.0% | 0.0% | 1.2% | 1.2% | 2.4% | 4.8% |
| (p-value vs placebo) | | (0.9962) | (0.9962) | (0.9962) | (0.9962) | (0.9962) | (0.9962) |

TABLE XCVIII
Study 1 - ACR responses at week 1-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 17.4% | 22.0% | 34.1% | 27.9% | 17.4% | 22.4% | 34.5% |
| (p-value vs placebo) | | (0.9378) | (0.0664) | (0.3908) | (0.9378) | (0.8540) | (0.0588) |
| ACR20 (LOCF) | 17.4% | 22.0% | 34.1% | 27.9% | 17.4% | 22.4% | 34.5% |
| (p-value vs placebo) | | (0.9378) | (0.0664) | (0.3908) | (0.9378) | (0.8540) | (0.0588) |
| ACR50 (NRI) | 1.2% | 2.4% | 5.9% | 4.7% | 3.5% | 7.1% | 7.1% |
| (p-value vs placebo) | | (0.5426) | (0.4037) | (0.5111) | (0.5426) | (0.3512) | (0.3373) |
| ACR50 (LOCF) | 1.2% | 2.4% | 5.9% | 4.7% | 3.5% | 7.1% | 7.1% |
| (p-value vs placebo) | | (0.5426) | (0.4047) | (0.5111) | (0.5426) | (0.3512) | (0.3373) |
| ACR70 (NRI) | 1.2% | 0.0% | 0.00% | 1.2% | 1.2% | 2.4% | 4.8% |
| (p-value vs placebo) | | (0.9962) | (0.9962) | (0.9962) | (0.9962) | (0.9962) | (0.9962) |
| ACR70 (LOCF) | 1.2% | 0.0% | 0.0% | 1.2% | 2.4% | 2.4% | 4.8% |
| (p-value vs placebo) | | (0.9962) | (0.9962) | (0.9962) | (0.9962) | (0.9962) | (0.9962) |

TABLE XCIX
Study 1 - ACR responses at week 2-12 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 26.7% | 37.8% | 47.1% | 48.8% | 29.1% | 41.2% | 54.8% |
| (p-value vs placebo) | | (0.2523) | (0.0208) | (0.0111) | (0.7798) | (0.1260) | (0.0012) |
| ACR20 (LOCF) | 26.7% | 39.0% | 45.9% | 48.8% | 27.9% | 40.0% | 53.6% |
| (p-value vs placebo) | | (0.1789) | (0.0337) | (0.0111) | (0.9186) | (0.1342) | (0.0021) |
| ACR50 (NRI) | 5.8% | 7.3% | 20.0% | 11.6% | 7.0% | 11.8% | 21.4% |
| (p-value vs placebo) | | (0.7974) | (0.0427) | (0.5105) | (0.7974) | (0.5474) | (0.0246) |
| ACR50 (LOCF) | 5.8% | 7.3% | 20.0% | 11.6% | 7.0% | 11.8% | 22.6% |
| (p-value vs placebo) | | (0.7968) | (0.0430) | (0.5130) | (0.7968) | (0.5486) | (0.0154) |
| ACR70 (NRI) | 1.2% | 2.4% | 7.1% | 5.8% | 2.3% | 5.9% | 3.6% |
| (p-value vs placebo) | | (0.5842) | (0.3489) | (0.4860) | (0.5842) | (0.5266) | (0.5842) |
| ACR70 (LOCF) | 1.2% | 2.4% | 7.1% | 5.8% | 2.3% | 5.9% | 4.8% |
| (p-value vs placebo) | | (0.5835) | (0.3499) | (0.3841) | (0.5835) | (0.3968) | (0.5762) |

TABLE C
Study 1 - ACR responses at week 2-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 26.7% | 37.8% | 47.1% | 48.8% | 29.1% | 41.2% | 54.8% |
| (p-value vs placebo) | | (0.2523) | (0.0208) | (0.0111) | (0.7798) | (0.1260) | (0.0012) |
| ACR20 (LOCF) | 27.9% | 39.0% | 48.2% | 48.8% | 29.1% | 41.2% | 56.0% |
| (p-value vs placebo) | | (0.2541) | (0.0213) | (0.0148) | (0.9179) | (0.1872) | (0.0012) |
| ACR50 (NRI) | 5.8% | 7.3% | 20.0% | 11.6% | 7.0% | 11.8% | 21.4% |
| (p-value vs placebo) | | (0.7974) | (0.0427) | (0.5105) | (0.7974) | (0.5474) | (0.0246) |
| ACR50 (LOCF) | 5.8% | 7.3% | 20.0% | 11.6% | 7.0% | 11.8% | 22.6% |
| (p-value vs placebo) | | (0.7963) | (0.0430) | (0.5130) | (0.7968) | (0.5486) | (0.0154) |
| ACR70 (NRI) | 1.2% | 2.4% | 7.1% | 5.8% | 2.3% | 5.9% | 3.6% |
| (p-value vs placebo) | | (0.5842) | (0.3489) | (0.4860) | (0.5842) | (0.5266) | (0.5842) |
| ACR70 (LOCF) | 1.2% | 2.4% | 7.1% | 5.8% | 2.3% | 5.9% | 4.8% |
| (p-value vs placebo) | | (0.5835) | (0.3499) | (0.3841) | (0.5835) | (0.3968) | (0.5762) |

TABLE CI
Study 1 - ACR responses at week 4-12 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 38.4% | 42.7% | 50.6% | 55.8% | 45.3% | 44.7% | 69.0% |
| (p-value vs placebo) | | (0.5890) | (0.4603) | (0.1021) | (0.5890) | (0.5890) | (0.0004) |
| ACR20 (LOCF) | 38.4% | 45.1% | 48.2% | 55.8% | 44.2% | 45.9% | 67.9% |
| (p-value vs placebo) | | (0.4782) | (0.4782) | (0.1024) | (0.4782) | (0.4782) | (0.0007) |
| ACR50 (NRI) | 7.0% | 12.2% | 32.9% | 17.4% | 15.1% | 17.6% | 35.7% |
| (p-value vs placebo) | | (0.2571) | (0.0003) | (0.1056) | (0.1943) | (0.1130) | (0.0001) |
| ACR50 (LOCF) | 7.0% | 12.2% | 32.9% | 16.3% | 16.3% | 20.0% | 34.5% |
| (p-value vs placebo) | | (0.2577) | (0.0003) | (0.1076) | (0.1270) | (0.0595) | (0.0002) |
| ACR70 (NRI) | 3.5% | 7.3% | 15.3% | 4.7% | 5.8% | 9.4% | 10.7% |
| (p-value vs placebo) | | (0.6646) | (0.0736) | (0.6646) | (0.6646) | (0.5107) | (0.3192) |
| ACR70 (LOCF) | 3.5% | 7.3% | 15.3% | 4.7% | 5.8% | 9.4% | 9.5% |
| (p-value vs placebo) | | (0.6635) | (0.0727) | (0.6635) | (0.6635) | (0.5106) | (0.4186) |

TABLE CII
Study 1 - ACR responses at week 4-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 38.4% | 42.7% | 51.8% | 55.8% | 45.3% | 45.3% | 67.9% |
| (p-value vs placebo) | | (0.5889) | (0.3333) | (0.1035) | (0.5889) | (0.5889) | (0.0007 |
| ACR20 (LOCF) | 39.5% | 45.1% | 51.8% | 55.8% | 46.5% | 47.1% | 67.9% |
| (p-value vs placebo) | | (0.4792) | (0.4586) | (0.1512) | (0.4792) | (0.4792) | (0.0012) |
| ACR50 (NRI) | 7.0% | 12.2% | 32.9% | 17.4% | 15.1% | 17.6% | 34.5% |
| (p-value vs placebo) | | (0.2568) | (0.0003) | (0.1054) | (0.1948) | (0.1127) | (0.0002) |
| ACR50 (LOCF) | 7.0% | 12.2% | 32.9% | 17.4% | 16.3% | 20.0% | 34.5% |
| (p-value vs placebo) | | (0.2567) | (0.0003) | (0.0973) | (0.1297) | (0.0609) | (0.0002) |
| ACR70 (NRI) | 3.5% | 7.3% | 15.3% | 4.7% | 5.8% | 9.4% | 10.7% |
| (p-value vs placebo) | | (0.6646) | (0.0736) | (0.6646) | (0.6646) | (0.5107) | (0.3192) |
| ACR70 (LOCF) | 3.5% | 7.3% | 15.3% | 4.7% | 5.8% | 9.4% | 10.7% |
| (p-value vs placebo) | | (0.6646) | (0.0736) | (0.6646) | (0.6646) | (0.5107) | (0.3192) |

TABLE CIII
Study 1 - ACR responses at week 8-12 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 45.3% | 54.9% | 63.5% | 68.6% | 58.1% | 54.1% | 75.0% |
| (p-value vs placebo) | | (0.2440) | (0.0720) | (0.0102) | (0.2440) | (0.2440) | (0.0006) |
| ACR20 (LOCF) | 46.5% | 57.3% | 63.5% | 68.6% | 58.1% | 55.3% | 75.0% |
| (p-value vs placebo) | | (0.2424) | (0.1064) | (0.0164) | (0.2424) | (0.2424) | (0.0010) |
| ACR50 (NRI) | 12.8% | 26.8% | 34.1% | 33.7% | 25.6% | 32.9% | 45.2% |
| (p-value vs placebo) | | (0.0393) | (0.0043) | (0.0043) | (0.0393) | (0.0064) | (<0.0001) |
| ACR50 (LOCF) | 11.6% | 25.6% | 35.3% | 33.7% | 25.6% | 32.9% | 45.2% |
| (p-value vs placebo) | | (0.0232) | (0.0018) | (0.0022) | (0.0232) | (0.0034) | (<0.0001) |
| ACR70 (NRI) | 7.0% | 11.0% | 22.4% | 18.6% | 9.3% | 14.1% | 27.4% |
| (p-value vs placebo) | | (0.6122) | (0.0324) | (0.0914) | (0.6122) | (0.4143) | (0.0041) |
| ACR70 (LOCF) | 7.0% | 11.0% | 23.5% | 18.6% | 8.1% | 14.1% | 26.2% |
| (p-value vs placebo) | | (0.7328) | (0.0197) | (0.0896) | (0.8139) | (0.4113) | (0.0066) |

TABLE CIV
Study 1 - ACR responses at week 8-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 45.3% | 54.9% | 64.7% | 69.8% | 58.1% | 54.1% | 75.0% |
| (p-value vs placebo) | | (0.2439) | (0.0462) | (0.0060) | (0.2438) | (0.2438) | (0.0006) |
| ACR20 (LOCF) | 46.5% | 57.3% | 64.7% | 69.8% | 59.3% | 55.3% | 75.0% |
| (p-value vs placebo) | | (0.2415) | (0.0690) | (0.0096) | (0.2415) | (0.2415) | (0.0010) |
| ACR50 (NRI) | 12.8% | 26.8% | 35.3% | 34.9% | 25.6% | 32.9% | 45.2% |
| (p-value vs placebo) | | (0.0393) | (0.0033) | (0.0034) | (0.0393) | (0.0064) | (<0.0001) |
| ACR50 (LOCF) | 12.8% | 26.8% | 35.3% | 34.9% | 25.6% | 32.9% | 45.2% |
| (p-value vs placebo) | | (0.0393) | (0.0033) | (0.0034) | 0.0393) | (0.0064) | (<0.0001) |
| ACR70 (NRI) | 7.0% | 11.0% | 23.5% | 18.6% | 9.3% | 14.1% | 27.4% |
| (p-value vs placebo) | | (0.6117) | (0.0201) | (0.0916) | (0.6117) | (0.4135) | (0.0041) |
| ACR70 (LOCF) | 7.0% | 11.0% | 23.5% | 18.6% | 9.3% | 14.1% | 27.4% |
| (p-value vs placebo) | | (0.6117) | (0.0201) | (0.0916) | (0.6117) | (0.4135) | (0.0041) |

TABLE CV
Study 1 - ACR responses at week 12-12 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 45.3% | 56.1% | 62.4% | 68.6% | 57.0% | 58.8% | 79.8% |
| (p-value vs placebo) | | (0.1670) | (0.1114) | (0.0103) | (0.1670) | (0.1670) | (<0.0001) |
| ACR20 (LOCF) | 44.2% | 59.8% | 63.5% | 70.9% | 58.1% | 58.8% | 79.8% |
| (p-value vs placebo) | | (0.0680) | (0.0468) | (0.0018) | (0.0680) | (0.0680) | (0.0001) |
| ACR50 (NRI) | 15.1% | 31.7% | 38.8% | 43.0% | 27.9% | 34.1% | 54.8% |
| (p-value vs placebo) | | (0.0240) | (0.0027) | (0.0004) | (0.0472) | (0.0133) | (<0.0001) |
| ACR50 (LOCF) | 15.1% | 31.7% | 38.8% | 44.2% | 29.1% | 34.1% | 56.0% |
| (p-value vs placebo) | | (0.0238) | (0.0025) | (0.0002) | (0.0313) | (0.0130) | (<0.0001) |
| ACR70 (NRI) | 8.1% | 15.9% | 20.0% | 24.4% | 14.0% | 18.8% | 31.0% |
| (p-value vs placebo) | | (0.2464) | (0.0928) | (0.0235) | (0.2464) | (0.1358) | (0.0020) |
| ACR70 (LOCF) | 8.1% | 15.9% | 22.4% | 24.4% | 14.0% | 18.8% | 29.8% |
| (p-value vs placebo) | | (0.2458) | (0.0500) | (0.0229) | (0.2458) | (0.1346) | (0.0033) |

TABLE CVI
Study 1 - ACR responses at week 12-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 45.3% | 56.1% | 62.4% | 69.8% | 57.0% | 58.8% | 79.8) |
| (p-value vs placebo) | | (0.1671) | (0.1116) | (0.0061) | (0.1671) | (0.1671) | (<0.0001) |
| ACR20 (LOCF) | 47.7% | 59.8% | 67.1% | 70.9% | 59.3% | 60.0% | 81.0% |
| (p-value vs placebo) | | (0.1292) | (0.0433) | (0.0087) | (0.1292) | (0.1292) | (<0.0001) |
| ACR50 (NRI) | 15.1% | 31.7% | 38.8% | 43.0% | 27.9% | 34.1% | 54.8% |
| (p-value vs placebo) | | (0.0240) | (0.0027) | (0.0004) | (0.0472) | (0.0133) | (<0.0001) |
| ACR50 (LOCF) | 15.1% | 31.7% | 41.2% | 44.2% | 29.1% | 34.1% | 57.1% |
| (p-value vs placebo) | | (0.0238) | (0.0009) | (0.0002) | (0.0315) | (0.0130) | (<0.0001) |
| ACR70 (NRI) | 8.1% | 15.9% | 20.0% | 24.4% | 14.0% | 18.8% | 31.0% |
| (p-value vs placebo) | | (0.2464) | (0.0928) | (0.0235) | (0.2464) | (0.1358) | (0.0020) |
| ACR70 (LOCF) | 8.1% | 15.9% | 22.4% | 24.4% | 14.0% | 18.8% | 31.0% |
| (p-value vs placebo) | | (0.2458) | (0.0501) | (0.0501) | (0.2458) | (0.1352) | (0.0020) |

At Week 12, the subjects on placebo who did not achieve at least a 20% improvement in swollen joint count (SJC66) and tender joint count (TJC68) were re-randomized automatically to receive Compound 1 (dosed as a [Compound 1:HCl:3H₂O]) either at 100 mg q.d. or 50 mg b.i.d. doses in a blinded fashion; subjects on 50 mg q.d. who did not achieve at least a 20% improvement in SJC66 and TJC68 were assigned to 100 mg q.d. and subjects on 25 mg b.i.d. who did not achieve a 20% improvement in SJC66 and TJC68 were assigned to 50 mg b.i.d. Subjects who switched treatment at week 12 were handled as if they discontinued at week 12 for the purpose of statistical analysis, whereas subjects in the other groups maintained their randomized treatment until Week 24. Consequently, the data reported from week 16 to week 24 only refers to the data for the subjects continuing on the same treatment course from week 0 to week 24.

TABLE CVII
Study 1 - ACR responses at week 16-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 39.5% | 56.1% | 64.7% | 73.3% | 52.3% | 64.7% | 82.1% |
| (p-value vs placebo) | | (0.0634) | (0.0034) | (<0.0001) | (0.0964) | (0.0027) | (<0.0001) |
| ACR20 (LOCF) | 43.0% | 59.8% | 69.4% | 74.4% | 55.8% | 67.1% | 84.5% |
| (p-value vs placebo) | | (0.0596) | (0.0022) | (0.0002) | (0.0972) | (0.0041) | (<0.0001) |
| ACR50 (NRI) | 19.8% | 37.8% | 52.9% | 48.8% | 30.2% | 31.8% | 61.9% |
| (p-value vs placebo) | | (0.0299) | (<0.0001) | (0.0003) | (0.1223) | (0.1223) | (<0.0001) |
| ACR50 (LOCF) | 20.9% | 37.8% | 55.3% | 50.0% | 31.4% | 32.9% | 64.3% |
| (p-value vs placebo) | | (0.0486) | (<0.0001) | (0.0003) | (0.1284) | (0.01284) | (<0.0001) |
| ACR70 (NRI) | 11.6% | 18.3% | 24.7% | 26.7% | 10.5% | 21.2% | 36.9% |
| (p-value vs placebo) | | (0.4572) | (0.1207) | (0.0566) | (0.7691) | (0.2868) | (0.0010) |
| ACR70 (LOCF) | 11.6% | 18.3% | 27.1% | 26.7% | 10.5% | 21.2% | 36.9% |
| (p-value vs placebo) | | (0.4573) | (0.0499) | (0.0446) | (0.7691) | (0.2859) | (0.0010) |

TABLE CVIII
Study 1 - ACR responses at week 20-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 38.4% | 57.3% | 69.4% | 73.3% | 52.3% | 65.9% | 84.5% |
| (p-value vs placebo) | | (0.0286) | (0.0003) | (<0.0001) | (0.0693) | (0.0010) | (<0.0001) |
| ACR20 (LOCF) | 44.2% | 61.0% | 76.5% | 76.7% | 58.1% | 68.2% | 88.1% |
| (p-value vs placebo) | | (0.0587) | (<0.0001) | (<0.0001) | (<0.0001) | (0.0042) | (<0.0001) |
| ACR50 (NRI) | 15.1% | 39.0% | 45.9% | 52.3% | 38.4% | 41.2% | 58.3% |
| (p-value vs placebo) | | (0.0008) | (0.0001) | (<0.0001) | (0.0008) | (0.0007) | (<0.0001) |
| ACR50 (LOCF) | 17.4% | 39.0% | 50.6% | 54.7% | 41.9% | 41.2% | 61.9% |
| (p-value vs placebo) | | (0.0021) | (<0.0001) | (<0.0001) | (0.0012) | (0.0015) | (<0.0001) |
| ACR70 (NRI) | 9.3% | 23.2% | 30.6% | 23.3% | 10.5% | 22.4% | 36.9% |
| (p-value vs placebo) | | (0.0341) | (0.0046) | (0.0340) | (0.8253) | (0.0454) | (0.0003) |
| ACR70 (LOCF) | 10.5% | 23.2% | 32.9% | 24.4% | 11.6% | 22.4% | 36.9% |
| (p-value vs placebo) | | (0.0604) | (0.0031) | (0.0525) | (0.8363) | (0.0788) | (0.0006) |

TABLE CIX
Study 1 - ACR responses at week 24-24 weeks results

| | | q.d. | | | b.i.d. | | |
|---|---|---|---|---|---|---|---|
| | | groups | | | groups | | |
| | Placebo | 50 mg | 100 mg | 200 mg | 2 × 25 mg | 2 × 50 mg | 2 × 100 mg |
| | (N = 86) | (N = 82) | (N = 85) | (N = 86) | (N = 86) | (N = 85) | (N = 84) |
| ACR20 (NRI) | 41.9% | 54.9% | 60.0% | 73.3% | 55.8% | 60.0% | 79.8% |
| (p-value vs placebo) | | (0.0953) | (0.0545) | (0.0002) | (0.0953) | (0.0507) | (<0.0001) |
| ACR20 (LOCF) | 45.3% | 61.0% | 72.9% | 77.9% | 62.8% | 67.1% | 86.9% |
| (p-value vs placebo) | | (0.0447) | (0.0011) | (<0.0001) | (0.0447) | (0.0123) | (<0.0001) |
| ACR50 (NRI) | 17.4% | 35.4% | 45.9% | 50.0% | 34.9% | 35.3% | 54.8% |
| (p-value vs placebo) | | (0.0105) | (0.0004) | (<0.0001) | (0.0105) | (0.0105) | (<0.0001) |
| ACR50 (LOCF) | 18.6% | 36.6% | 54.1% | 54.7% | 38.4% | 38.8% | 61.9% |
| (p-value vs placebo) | | 0.0101 | (<0.0001) | (<0.0001) | (0.0094) | (0.0078) | (<0.0001) |
| ACR70 (NRI) | 9.3% | 22.0% | 32.9% | 29.1% | 20.9% | 23.5% | 39.3% |
| (p-value vs placebo) | | (0.0391) | (0.0017) | (0.0060) | (0.0391) | (0.0391) | (0.0001) |
| ACR70 (LOCF) | 9.3% | 22.0% | 37.6% | 31.4% | 22.1% | 24.7% | 42.9% |
| (p-value vs placebo) | | (0.0267) | (0.0002) | (0.0021) | (0.0267) | (0.0267) | (<0.0001) |

TABLE CX
Study 1 - Subject VAS mean changes - 12 weeks results (ACR patient assessment)

| | | | | | b.i.d. | | |
|---|---|---|---|---|---|---|---|
| | | q.d. groups | | | groups | | |
| | Placebo | 50 mg | 100 mg | 200 mg | 2 × 25 mg | 2 × 50 mg | 2 × 100 mg |
| Week, LOCF | (N = 86) | (N = 82) | (N = 85) | (N = 86) | (N = 86) | (N = 85) | (N = 84) |
| 0 | 0.0 | 0.0 | 0.0 | 0.0 | 0.0 | 0.0 | 0.0 |
| 1 | −8.9 | −11.2 | −13.2 | −16.7 | −8.2 | −8.8 | −20.0 |
| 2 | −10.6 | −15.5 | −20.8 | −21.8 | −11.7 | −13.6 | −25.5 |
| 4 | −13.2 | −20.9 | −24.4 | −25.2 | −16.0 | −16.9 | −28.6 |
| 8 | −16.1 | −26.4 | −29.8 | −29.6 | −22.2 | −21.0 | −32.8 |
| 12 | −16.7 | −25.2 | −29.1 | −34.0 | −25.2 | −27.0 | −35.6 |

TABLE CXI
Study 1 - Subject VAS mean changes - 24 weeks results (ACR patient assessment)

| | | | | | b.i.d. | | |
|---|---|---|---|---|---|---|---|
| | | q.d. groups | | | groups | | |
| | Placebo | 50 mg | 100 mg | 200 mg | 2 × 25 mg | 2 × 50 mg | 2 × 100 mg |
| Week, LOCF | (N = 86) | (N = 82) | (N = 85) | (N = 86) | (N = 86) | (N = 85) | (N = 84) |
| 0 | 0.0 | 0.0 | 0.0 | 0.0 | 0.0 | 0.0 | 0.0 |
| 1 | −8.9 | −11.2 | −12.9 | −16.7 | −8.2 | −8.7 | −20 |
| 2 | −10.6 | −15.5 | −20.9 | −21.8 | −11.7 | −13.8 | −25.5 |
| 4 | −13.2 | −20.9 | −24.4 | −24.9 | −16.0 | −16.9 | −28.2 |
| 8 | −16.1 | −26.4 | −29.8 | −29.6 | −22.2 | −21.0 | −33.0 |
| 12 | −16.7 | −25.2 | −29.1 | −34.2 | −25.2 | −27.0 | −35.6 |
| 16 | −19 | −27.7 | −31.7 | −32.9 | −25.5 | −26.3 | −37.9 |
| 20 | −18.2 | −28.1 | −35.1 | −33.0 | −25.6 | −29.7 | −39.1 |
| 24 | −17.9 | −29.4 | −34.4 | −34.9 | −27.3 | −28.1 | −39.1 |

TABLE CXII
Study 2 - ACR responses at week 1 - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 16.7% | 22.2% | 24.3% | 39.1% |
| (p-value vs | | (0.3900) | (0.3900) | (0.0052) |
| placebo) | | | | |
| ACR20 (LOCF) | 16.7% | 20.8 | 24.3% | 39.1% |
| (p-value vs | | (0.5153) | (0.5113) | (0.0054) |
| placebo) | | | | |
| ACR50 (NRI) | 1.4% | 1.4% | 10.0% | 7.2% |
| (p-value vs | | (0.9974) | (0.1651) | (0.2304) |
| placebo) | | | | |
| ACR50 (LOCF) | 1.4% | 1.4% | 10.0% | 7.2% |
| (p-value vs | | (0.9974) | (0.1651) | (0.2304) |
| placebo) | | | | |
| ACR70 (NRI) | 0.0% | 0.0% | 1.4% | 1.4% |
| (p-value vs | | (0.9988) | (0.9988) | (0.9988) |
| placebo) | | | | |
| ACR70 (LOCF) | 0.0% | 0.0% | 1.4% | 1.4% |
| (p-value vs | | (0.9988) | (0.9988) | (0.9988) |
| placebo) | | | | |

TABLE CXIII
Study 2 - ACR responses at week 1 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 16.7% | 22.2% | 24.3% | 39.1% |
| (p-value vs | | (0.3900) | (0.3900) | (0.0052) |
| placebo) | | | | |
| ACR20 (LOCF) | 16.7% | 22.2 | 24.3% | 39.1% |
| (p-value vs | | (0.5153) | (0.5113) | (0.0054) |
| placebo) | | | | |
| ACR50 (NRI) | 1.4% | 1.4% | 10.0% | 7.2% |
| (p-value vs | | (0.9974) | (0.1651) | (0.2304) |
| placebo) | | | | |
| ACR50 (LOCF) | 1.4% | 1.4% | 10.0% | 7.2% |
| (p-value vs | | (0.9974) | (0.1651) | (0.2304) |
| placebo) | | | | |
| ACR70 (NRI) | 0.0% | 0.0% | 1.4% | 1.4% |
| (p-value vs | | (0.9988) | (0.9988) | (0.9988) |
| placebo) | | | | |
| ACR70 (LOCF) | 0.0% | 0.0% | 1.4% | 1.4% |
| (p-value vs | | (0.9988) | (0.9988) | (0.9988) |
| placebo) | | | | |

TABLE CXIV
Study 2 - ACR responses at week 2 - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 22.2% | 34.7% | 32.9% | 56.5% |
| (p-value vs | | (0.1542) | (0.1542) | (<0.0001) |
| placebo) | | | | |
| ACR20 (LOCF) | 20.8% | 33.3% | 34.3% | 53.6% |
| (p-value vs | | (0.0864) | (0.0864) | (0.0001) |
| placebo) | | | | |
| ACR50 (NRI) | 4.2% | 5.6% | 7.1% | 21.7% |
| (p-value vs | | (0.7241) | (0.7241) | (0.0050) |
| placebo) | | | | |
| ACR50 (LOCF) | 4.2% | 5.6% | 8.6% | 21.7% |
| (p-value vs | | (0.7246) | (0.5189) | (0.0045) |
| placebo) | | | | |
| ACR70 (NRI) | 2.8% | 1.4% | 1.4% | 4.3% |
| (p-value vs | | (0.5865) | (0.5865) | (0.5865) |
| placebo) | | | | |
| ACR70 (LOCF) | 2.8% | 1.4% | 1.4% | 4.3% |
| (p-value vs | | (0.5865) | (0.5865) | (0.5865) |
| placebo) | | | | |

TABLE CXV
Study 2 - ACR responses at week 2 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 22.2% | 33.3% | 32.9% | 56.5% |
| (p-value vs | | (0.1533) | (0.1533) | (<0.0001) |
| placebo) | | | | |
| ACR20 (LOCF) | 22.2% | 33.3% | 35.7% | 56.5% |
| (p-value vs | | (0.1312) | (0.1312) | (0.0001) |
| placebo) | | | | |
| ACR50 (NRI) | 4.2% | 5.6% | 7.1% | 21.7% |
| (p-value vs | | (0.7241) | (0.7241) | (0.0050) |
| placebo) | | | | |
| ACR50 (LOCF) | 4.2% | 5.6% | 8.6% | 21.7% |
| (p-value vs | | (0.7246) | (0.5189) | (0.0045) |
| placebo) | | | | |
| ACR70 (NRI) | 2.8% | 1.4% | 1.4% | 4.3% |
| (p-value vs | | (0.5865) | (0.5865) | (0.5865) |
| placebo) | | | | |
| ACR70 (LOCF) | 2.8% | 1.4% | 1.4% | 4.3% |
| (p-value vs | | (0.5865) | (0.5865) | (0.5865) |
| placebo) | | | | |

TABLE CXVI
Study 2 - ACR responses at week 4 - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 27.6% | 54.2% | 44.3% | 63.8% |
| (p-value vs | | (0.0024) | (0.0404) | (<0.0001) |
| placebo) | | | | |
| ACR20 (LOCF) | 27.8% | 54.2% | 44.3% | 62.3% |
| (p-value vs | | (0.0024) | (0.403) | (<0.0001) |
| placebo) | | | | |
| ACR50 (NRI) | 5.6% | 15.3% | 18.6% | 23.2% |
| (p-value vs | | (0.0557) | (0.0263) | (0.0053) |
| placebo) | | | | |
| ACR50 (LOCF) | 5.6% | 13.9% | 18.6% | 23.2% |
| (p-value vs | | (0.0909) | (0.0252) | (0.0050) |
| placebo) | | | | |
| ACR70 (NRI) | 1.4% | 8.3% | 5.7% | 11.6% |
| (p-value vs | | (0.1536) | (0.1741) | (0.0605) |
| placebo) | | | | |
| ACR70 (LOCF) | 1.4% | 8.3% | 5.7% | 11.6% |
| (p-value vs | | (0.1536) | (0.1741) | (0.0605) |
| placebo) | | | | |

TABLE CXVII
Study 2 - ACR responses at week 4 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 27.6% | 52.8% | 44.3% | 62.3% |
| (p-value vs | | (0.0041) | (0.0399) | (<0.0001) |
| placebo) | | | | |
| ACR20 (LOCF) | 27.8% | 52.8% | 44.3% | 63.8% |
| (p-value vs | | (0.0040) | (0.0389) | (<0.0001) |
| placebo) | | | | |
| ACR50 (NRI) | 5.6% | 15.3% | 18.6% | 23.2% |
| (p-value vs | | (0.0557) | (0.0263) | (0.0053) |
| placebo) | | | | |
| ACR50 (LOCF) | 5.6% | 15.3% | 18.6% | 23.2% |
| (p-value vs | | (0.0557) | (0.0263) | (0.0053) |
| placebo) | | | | |
| ACR70 (NRI) | 1.4% | 8.3% | 5.7% | 11.6% |
| (p-value vs | | (0.1536) | (0.1741) | (0.0605) |
| placebo) | | | | |
| ACR70 (LOCF) | 1.4% | 8.3% | 5.7% | 11.6% |
| (p-value vs | | (0.1536) | (0.1741) | (0.0605) |
| placebo) | | | | |

TABLE CXVIII
Study 2 - ACR responses at week 8-12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 26.4% | 61.1% | 61.4% | 73.9% |
| (p-value vs | | (<0.0001) | (<0.0001) | (<0.0001) |
| placebo) | | | | |
| ACR20 (LOCF) | 27.8% | 62.5% | 60.0% | 72.5% |
| (p-value vs | | (<0.0001) | (0.0001) | (<0.0001) |
| placebo) | | | | |
| ACR50 (NRI) | 5.6% | 16.7% | 25.7% | 33.3% |
| (p-value vs | | (0.0389) | (0.0028) | (0.0002) |
| placebo) | | | | |
| ACR50 (LOCF) | 5.6% | 16.7% | 25.7% | 33.3% |
| (p-value vs | | (0.0389) | (0.0028) | (0.0002) |
| placebo) | | | | |
| ACR70 (NRI) | 2.8% | 6.9% | 11.4% | 17.4% |
| (p-value vs | | (0.2637) | (0.0923) | (0.0148) |
| placebo) | | | | |
| ACR70 (LOCF) | 2.8% | 6.9% | 11.4% | 17.4% |
| (p-value vs | | (0.2637) | (0.0923) | (0.0148) |
| placebo) | | | | |

TABLE CXIX
Study 2 - ACR responses at week 8 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 26.4% | 61.1% | 61.4% | 73.9% |
| (p-value vs | | (<0.0001) | (<0.0001) | (<0.0001) |
| placebo) | | | | |
| ACR20 (LOCF) | 27.8% | 61.1% | 61.4% | 73.9% |
| (p-value vs | | (<0.0001) | (0.0001) | (<0.0001) |
| placebo) | | | | |
| ACR50 (NRI) | 5.6% | 16.7% | 25.7% | 33.3% |
| (p-value vs | | (0.0382) | (0.0026) | (0.0002) |
| placebo) | | | | |
| ACR50 (LOCF) | 5.6% | 16.7% | 25.7% | 33.3% |
| (p-value vs | | (0.0382) | (0.0026) | (0.0002) |
| placebo) | | | | |
| ACR70 (NRI) | 2.8% | 6.9% | 11.4% | 17.4% |
| (p-value vs | | (0.2637) | (0.0923) | (0.0148) |
| placebo) | | | | |
| ACR70 (LOCF) | 2.8% | 6.9% | 11.4% | 17.4% |
| (p-value vs | | (0.2637) | (0.0923) | (0.0148) |
| placebo) | | | | |

TABLE CXX
Study 2 - ACR responses at week 12 - 12 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI)% | 30.6% | 66.7%% | 65.7%% | 72.5%% |
| (p-value vs | | (<0.0001) | (<0.0001) | (<0.0001) |
| placebo) | | | | |
| ACR20 (LOCF)% | 29.2% | 68.1%% | 65.7%% | 72.5%% |
| (p-value vs | | (<0.0001) | (<0.0001) | (<0.0001) |
| placebo) | | | | |
| ACR50 (NRI)% | 11.1% | 36.1%% | 34.3%% | 43.5%% |
| (p-value vs | | (0.0011) | (0.0013) | (<0.0001) |
| placebo) | | | | |
| ACR50 (LOCF)% | 11.1% | 36.1%% | 32.9%% | 42.0%% |
| (p-value vs | | (0.0011) | (0.0021) | (0.0001) |
| placebo) | | | | |
| ACR70 (NRI)% | 4.2% | 8.3%% | 18.6%% | 13.0%% |
| (p-value vs | | (0.3022) | (0.0219) | (0.0885) |
| placebo) | | | | |
| ACR70 (LOCF)% | 4.2% | 8.3%% | 18.6%% | 13.0%% |
| (p-value vs | | (0.3022) | (0.0219) | (0.0885) |
| placebo) | | | | |

TABLE CXXI
Study 2 - ACR responses at week 12 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | 29.2% | 66.7% | 67.1% | 72.5% |
| (p-value vs | | (<0.0001) | (<0.0001) | (<0.0001) |
| placebo) | | | | |
| ACR20 (LOCF) | 30.6% | 66.7% | 67.1% | 72.5% |
| (p-value vs | | (<0.0001) | (<0.0001) | (<0.0001) |
| placebo) | | | | |
| ACR50 (NRI) | 11.1% | 34.7% | 35.7% | 43.5% |
| (p-value vs | | (0.0009) | (0.0009) | (<0.0001) |
| placebo) | | | | |
| ACR50 (LOCF) | 11.1% | 34.7% | 35.7% | 43.5% |
| (p-value vs | | (0.0009) | (0.0009) | (0.0001) |
| placebo) | | | | |
| ACR70 (NRI) | 4.2% | 8.3% | 18.6% | 13.0% |
| (p-value vs | | (0.3022) | (0.0219) | (0.0885) |
| placebo) | | | | |
| ACR70 (LOCF) | 4.2% | 8.3% | 18.6% | 13.0% |
| (p-value vs | | (0.3022) | (0.0219) | (0.0885) |
| placebo) | | | | |

At Week 12, all subjects on placebo and the subjects on the 50 mg dose who did not achieve at least a 20% improvement in swollen joint count (SJC66) and tender joint count (TJC68) were assigned to 100 mg q.d. in a blinded fashion and continued treatment until Week 24. Subjects in the other groups maintained their randomized treatment until Week 24. Consequently, at week 12, there are no subjects on placebo, and the data reported from week 16 to week 24 only refers to the data for the subjects continuing on the same treatment course from week 0 to week 24.

TABLE CXXII
Study 2 - ACR responses at week 16 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | — | 55.6% | 70.0 | 73.9 |
| ACR20 (LOCF) | — | 61.1% | 71.4 | 73.9 |
| ACR50 (NRI) | — | 33.3% | 38.6% | 40.6% |
| ACR50 (LOCF) | — | 36.1% | 40.0% | 40.6% |
| ACR70 (NRI) | — | 15.3% | 17.1% | 18.8% |
| ACR70 (LOCF) | — | 15.3% | 17.1% | 18.8% |

TABLE CXXIII
Study 2 - ACR responses at week 20 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | — | 51.4% | 70.0% | 75.4% |
| ACR20 (LOCF) | — | 58.3% | 72.9% | 75.4% |
| ACR50 (NRI) | — | 27.8% | 41.4% | 44.9% |
| ACR50 (LOCF) | — | 33.3% | 42.9% | 44.9% |
| ACR70 (NRI) | — | 12.5% | 24.3% | 24.6% |
| ACR70 (LOCF) | — | 13.9% | 25.7% | 24.6% |

TABLE CXXIV
Study 2 - ACR responses at week 24 - 24 weeks results

| | Placebo | 50 mg q.d. | 100 mg q.d. | 200 mg q.d. |
|---|---|---|---|---|
| Weeks | (N = 72) | (N = 72) | (N = 70) | (N = 69) |
| ACR20 (NRI) | — | 56.9% | 77.1% | 68.1% |
| ACR20 (LOCF) | — | 65.3% | 80.0% | 73.9% |
| ACR50 (NRI) | — | 31.9% | 40.0% | 44.9% |
| ACR50 (LOCF) | — | 36.1% | 41.4% | 49.3% |
| ACR70 (NRI) | — | 19.4% | 25.7% | 24.6% |
| ACR70 (LOCF) | — | 19.4% | 25.7% | 27.5% |

TABLE CXXV
Study 2 - Subject VAS mean changes - 12
weeks results (ACR patient assessment)

| Week, | Placebo % | 50 mg q.d. % | 100 mg q.d. % | 200 mg q.d. % |
|---|---|---|---|---|
| LOCF | (N = 72) | (N = 72) | (N = 70) | (N = 69) |
| 0 | 0.0 | 0.0 | 0.0 | 0.0 |
| 1 | −5.5 | −12.3 | −12.2 | −14.8 |
| 2 | −9.6 | −17.3 | −17.2 | −19.9 |
| 4 | −10.8 | −19.3 | −21.3 | −25.0 |
| 8 | −14.0 | −23.6 | −29.2 | −30.3 |
| 12 | −11.5 | −27.4 | −29.8 | −28.8 |

### 3.8. Crohn's Disease Activity Index (CDAI)%

The Crohn's disease activity index (CDAI) is a numerical calculation derived from the sum of products from a list of 8 items (see Table CXXVI below), and multiplied by weighting factors for each item to define the severity of “disease activity” in patients with Crohn's disease (CD)_{[1]}. Essentially, the CDAI represents a numerical estimation of a physician's interpretation of patient symptoms. Index values of 150 and below are associated with quiescent or non-active disease (i.e. “remission”). Values over 150 are indicative of active disease, and over 450, extremely severe disease.%

In the present study, clinical remission is defined as a CDAI score of <150 points, and a clinical response is defined as a decrease in CDAI score of at least 100 points.

TABLE CXXVI
CDAI calculation components

| Category | Count | Factor |
|---|---|---|
| Number of liquid or | 7-day total number of liquid or very soft stools (reported on the 7 | ×2 |
| very soft stools | days immediately prior to the study visit) | |
| Abdominal pain | 7-day total of daily abdominal pain scores on a 3-point scale: | ×5 |
| | 0 = none, | |
| | 1 = mild, | |
| | 2 = moderate, | |
| | 3 = severe | |
| | (reported on the 7 days immediately prior to the study visit) | |
| General well being | 7-day total of daily general well-being scores on a 4-point scale: | ×7 |
| | 0 = well, 1 = slightly below par, 2 = poor, 3 = very poor, 4 = terrible | |
| | (reported on the 7 days immediately prior to the study visit) | |
| Extra-intestinal | Total number of checked boxes (check all that apply): | ×20 |
| manifestations of | Arthritis/arthralgia | |
| Crohn's | Iritis/uveitis | |
| Disease | Erythema nodosum/pyoderma | |
| | gangrenosum/aphthous stomatitis | |
| | Anal fissure, fistula, or abscess | |
| | Other fistula | |
| | Fever over 37.8° C. during past week | |
| Lomotil/Imodium/ | Yes = 1 | ×30 |
| opiates for diarrhea | No = 0 | |
| Abdominal mass | None = 0 | ×10 |
| | Questionable = 2 | |
| | Definite = 5 | |
| Hematocrit (%) | Males: subtract value from 47 | ×6 |
| | Females: subtract value from 42 | |
| Body Weight | $\frac{( Standard weight ( kg ) - Actual body weight ( kg )}{Standard body weight ( kg )} \times 100$ | ×1 |

TABLE CXXVII
Study 3 - 10 week CDAI scores - Overall

| | Placebo | 200 mg |
|---|---|---|
| | (N = 44) | (N = 128) |
| Clinical Remission (CDAI < 150), | 23% | 48% |
| NRI (p-value vs placebo) | | (0.0067) |
| Clinical Remission (CDAI < 150), | 30% | 50% |
| LOCF, (p-value vs placebo) | | (0.0288) |
| 100 point clinical response | 41% | 60% |
| (CDAI improvement ≥ 100 points), | | (0.0386) |
| NRI, (p-value vs placebo) | | |
| 100 point clinical response | 50% | 66% |
| (CDAI improvement ≥ 100 points), | | (0.0954) |
| LOCF, (p-value vs placebo) | | |
| 70 point clinical response | 55% | 66% |
| (CDAI improvement ≥ 100 points), | | (0.2473) |
| NRI, (p-value vs placebo) | | |
| 70 point clinical response | 64% | 71% |
| (CDAI improvement ≥ 100 points), | | (0.4650) |
| LOCF, (p-value vs placebo) | | |

TABLE CXXVIII
Study 3 - 10 week CDAI scores by prior TNF therapy

| | | | Anti-TNF experienced | |
|---|---|---|---|---|
| | Anti-TNF Naïve | | non-responders | |
| | Placebo | 200 mg | Placebo | 200 mg |
| | (N = 16) | (N = 57) | (N = 28) | (N = 71) |
| Clinical Remission (CDAI < 150), LOCF, (p-value | 25% | 65% | 32% | 38% |
| vs placebo) | | | | |
| 100 point clinical response (CDAI improvement ≥ | 63% | 74% | 43% | 59% |
| 100 points), LOCF, (p-value vs placebo) | | | | |
| 70 point clinical response (CDAI improvement ≥ | 81% | 81% | 54% | 63% |
| 100 points), LOCF, (p-value vs placebo) | | | | |

TABLE CXXIX
Study 3 - 10 week CDAI scores by screening CRP Level,, LOCF

| | CRP ≤ 10 ml/L | | CRP ≥ 10 mg/L | |
|---|---|---|---|---|
| | Placebo | 200 mg | Placebo | 200 mg |
| | (N = 25) | (N = 74) | (N = 19) | (N = 54) |
| Clinical Remission (CDAI < 150) | 36% | 46% | 21% | 56% |
| 100 point clinical response (CDAI improvement ≥ | 56% | 66% | 42% | 65% |
| 100 points) | | | | |
| 70 point clinical response (CDAI improvement ≥ | 76% | 73% | 47% | 69% |
| 100 points) | | | | |

TABLE CXXX
Study 3 - CDAI scores by baseline corticosteroid use, LOCF

| | With steroids | | Without steroids | |
|---|---|---|---|---|
| | Placebo | 200 mg | Placebo | 200 mg |
| | (N = 23) | (N = 63) | (N = 21) | (N = 65) |
| Clinical Remission (CDAI < 150) | 17% | 52% | 43% | 48% |
| 100 point clinical response (CDAI | 39% | 60% | 61% | 71% |
| improvement ≥ 100 points) | | | | |
| 70 point clinical response (CDAI | 57% | 70% | 71% | 72% |
| improvement ≥ 100 points) | | | | |

TABLE CXXXI
Study 3 - 20 week CDAI scores - Initial 200 mg responders

| Initial 200 responders | | |
|---|---|---|
| To placebo | To 100 mg | Continued 200 mg |
| (N = 14) | (N = 30) | (N = 30) |

| | Week 10 | Week 20 | Week 10 | Week 20 | Week 10 | Week 20 |
|---|---|---|---|---|---|---|
| Clinical Remission (CDAI <150), | 71% | 71% | 87% | 60% | 63% | 50% |
| NRI | | | | 0.4517 | | 0.2102 |
| (p-value vs placebo) | | | | | | 0.5133 |
| (p-value vs 100 mg) | | | | | | |
| Clinical Remission (CDAI <150), | 79% | 79% | 87% | 63% | 67% | 57% |
| LOCF | | | | 0.2965 | | 0.1776 |
| (p-value vs placebo) | | | | | | 0.6807 |
| (p-value vs 100 mg) | | | | | | |
| Clinical Remission (CDAI <150), | 77% | 77% | 87% | 75% | 70% | 58% |
| OC | | | | | | |
| Clinical Response (CDAI ≥100), | 86% | 79% | 97% | 73% | 83% | 67% |
| NRI | | | | 0.7341 | | 0.4959 |
| (p-value vs placebo) | | | | | | 0.6608 |
| (p-value vs 100 mg) | | | | | | |
| Clinical Response (CDAI ≥100), | 93% | 86% | 97% | 80% | 93% | 77% |
| LOCF | | | | 0.7003 | | 0.3619 |
| (p-value vs placebo) | | | | | | 0.8457 |
| (p-value vs 100 mg) | | | | | | |
| Clinical Remission (CDAI ≥100), | 92% | 85% | 97% | 92% | 93% | 77% |
| OC | | | | | | |
| Clinical Response (CDAI ≥70), | 93% | 86% | 100% | 77% | 90% | 70% |
| NRI | | | | 0.5158 | | 0.3316 |
| (p-value vs placebo) | | | | | | 0.6669 |
| (p-value vs 100 mg) | | | | | | |
| Clinical Response (CDAI ≥70), | 100% | 93% | 100% | 83% | 100% | 80% |
| LOCF | | | | 0.4406 | | 0.3619 |
| (p-value vs placebo) | | | | | | 0.8457 |
| (p-value vs 100 mg) | | | | | | |
| Clinical Response (CDAI ≥70), | 100% | 92% | 100% | 96% | 100% | 81% |
| OC | | | | | | |

TABLE CXXXII
Study 3 - 20 week CDAI scores - Initial placebo responders

| | Initial placebo responders | |
|---|---|---|
| | continuing on placebo | |
| | (N = 15) | |
| | Week 10 | Week 20 |
| Clinical Remission (CDAI < 150), NRI | 53% | 60% |
| Clinical Remission (CDAI < 150), LOCF | 73% | 67% |
| Clinical Remission (CDAI < 150), OC | 73% | 75% |
| Clinical Response (CDAI ≥ 100), NRI | 73% | 67% |
| Clinical Response (CDAI ≥ 100), LOCF | 100% | 87% |
| Clinical Response (CDAI ≥ 100), OC | 100% | 83% |
| Clinical Response (CDAI ≥ 70), NRI | 73% | 73% |
| Clinical Response (CDAI ≥ 70), LOCF | 100% | 93% |
| Clinical Response (CDAI ≥ 70), OC | 100% | 92% |

TABLE CXXXIII
Study 3 - 20 week CDAI scores - Initial 200 mg non-responders

| | Initial 200 mg non-responders | | | | |
|---|---|---|---|---|---|
| | To placebo | | Continued 200 mg | | |
| | (N = 9) | | (N = 25) | | |
| | Week 10 | Week 20 | Week 10 | Week 20 | p-value |
| Clinical Remission (CDAI < 150), NRI | 0% | 33% | 16% | 24% | 0.3529 |
| Clinical Remission (CDAI < 150), LOCF | 0% | 33% | 16% | 28% | 0.5079 |
| Clinical Remission (CDAI < 150), OC | 0% | 38% | 16% | 30% | NA |
| Clinical Response (CDAI ≥ 100), NRI | 11% | 33% | 32% | 32% | 0.6352 |
| Clinical Response (CDAI ≥ 70), NRI | 11% | 44% | 44% | 40% | 0.4841 |
| Clinical Response (CDAI ≥ 100), LOCF | 22% | 33% | 32% | 36% | 0.8123 |
| Clinical Response (CDAI ≥ 70), LOCF | 22% | 44% | 44% | 44% | 0.6354 |
| Clinical Response (CDAI ≥ 100), OC | 13% | 38% | 32% | 40% | NA |
| Clinical Response (CDAI ≥ 70), OC | 13% | 50% | 44% | 50% | NA |

TABLE CXXXIV
Study 3 - 20 week CDAI scores - Placebo
non-responders switching to 100 mg

| | Initial placebo non- |
|---|---|
| | responders switching |
| | to 100 mg at week 10 |
| | (N = 22) Week 20 |
| Clinical Remission (CDAI < 150), NRI | 32% |
| Clinical Remission (CDAI < 150), LOCF | 32% |
| Clinical Remission (CDAI < 150), OC | 33% |
| Clinical Response (CDAI ≥ 100), NRI | 59% |
| Clinical Response (CDAI ≥ 100), LOCF | 59% |
| Clinical Response (CDAI ≥ 100), OC | 62% |
| Clinical Response (CDAI ≥ 70), NRI | 68% |
| Clinical Response (CDAI ≥ 70), LOCF | 68% |
| Clinical Response (CDAI ≥ 70), OC | 71% |

### 3.9. CDAI Component—PRO2 Score

Within the CDAI score measurement parameters, the PRO2 score, based on the stool frequency (SF) and abdominal pain (AP) components of the CDAI, was also measured. The PRO2 score is calculated as follows: PRO2=7 x (mean daily number of liquid or very soft stools)+7×(mean daily abdominal pain score). This score provides a measurement of the symptoms improvement of the patient.

TABLE CXXXV
Study 3 - PRO2 score at week 10 (LOCF)

| | Placebo (N = 44) | 200 mg (N = 128) | p-values |
|---|---|---|---|
| PRO2 mean, baseline | 56.4 | 52.4 | |
| PRO2 mean CFB, W 10 | −15.8 | −21.9 | 0.0321 |

TABLE CXXXVI
Study 3 - derived PRO2 response at week 10 (NRI)

| | Placebo | 200 mg | |
|---|---|---|---|
| | (N = 44) | (N = 128) | p-values |
| PRO2 response | 20% | 34% | 0.1316 |
| (mean daily number of liquid | | | |
| or very soft stools ≤ 3, | | | |
| mean daily abdominal pain ≤ 1) | | | |
| PRO2 remission | 30% | 50% | 0.338 |
| (PRO2 ≤ 28) | | | |

TABLE CXXXVII
Study 3 - derived PRO2 response at week 10 (LOCF)

| | Placebo | 200 mg | |
|---|---|---|---|
| | (N = 44) | (N = 128) | p-values |
| PRO2 response | 23% | 38% | 0.1186 |
| (mean daily number of liquid | | | |
| or very soft stools ≤ 3, | | | |
| mean daily abdominal pain ≤ 1) | | | |
| PRO2 remission | 32% | 55% | 0.0140 |
| (PRO2 ≤ 28) | | | |

### 3.10. Inflammatory Bowel Disease Questionnaire (IBDQ)

The Inflammatory Bowel Disease Questionnaire (IBDQ) is a questionnaire for health-related quality of life assessment in patients with inflammatory bowel diseases: ulcerative colitis and Crohn's disease. It consists of 32 questions divided into four groups: bowel symptoms (10 items), systemic symptoms (5 items), emotional function (12 items) and social function (5 items). Every question has graded responses from 1 to 7, and thus the total score is ranging from 32 to 224 with higher scores representing better quality of life. The IBDQ is a validated assessment tool that reflects important changes in the quality of life of patients with IBD (adapted from Pallis et al, Inflamm Bowel Dis, Volume 10, Number 3, May 2004)

TABLE CXXXVIII
Study 3 - 10 weeks IBDQ scores (LOCF)

| | Placebo (N = 44) | | 200 mg (N = 128) | | |
|---|---|---|---|---|---|
| | Baseline | Week 10 | Baseline | Week 10 | p-value |
| IBDQ mean | 120.77 | 17.56 | 123.00 | 33.82 | 0.0045 |
| CFB | | | | | |
| Bowel | 39.09 | 5.59 | 41.07 | 9.96 | 0.0042 |
| symptoms | | | | | |
| Systemic | 15.68 | 2.93 | 16.32 | 5.73 | 0.0043 |
| symptoms | | | | | |
| Emotional | 45.66 | 6.10 | 45.91 | 12.07 | 0.0091 |
| status | | | | | |
| Social | 20.34 | 2.94 | 19.26 | 6.19 | 0.0188 |
| functioning | | | | | |

### 3.11. Endoscopic Scores for Crohn's Disease

For the evaluation of disease severity the Crohn's Disease Index of Severity (CDEIS), and the Simple Endoscopic Score for Crohn's Disease (SES-CD) may be used. These are validated scores for the measurement of endoscopic findings, (Sipponen et al, 2010).

### 3.11.1. CDEIS

For the grading of endoscopic findings, the bowel is divided into five segments: the terminal ileum, the right, transverse, left colon, and rectum. The ileum is scored for the full examined extent. The right colonic segment included the cecum, the ileocecal valve, and the ascending colon to the hepatic flexure. The bowel segment between hepatic and splenic flexures was the transverse colon. The left colon included both the descending colon and the sigmoid. The rectum was the segment distal to the rectosigmoid junction. For the CDEIS, as originally defined, presence of mucosal superficial ulcers, presence of deep ulcers, the extent of surface involved by disease, the extent of ulcerated surface, and the presence of ulcerated or nonulcerated stenosis are recorded in each segment.₇ The CDEIS score can range from 0-44, with a higher score indicating more severe disease. A CDEIS below 3 is classified as inactive, 3-9 is mildly active, 9-12 is moderately active, and >12 is severely active disease.

### 3.11.2. SES-CD

For the SES-CD, four endoscopic variables in the five segments are scored from 0-3: Variable “presence and size of ulcers” is scored 0 when no ulcers are present, small ulcers (diameter 0.1-0.5 cm) are scored 1, medium-sized ulcers (diameter 0.5-2 cm) 2, and large ulcers (>2 cm) 3. Variable “extent of ulcerated surface” is scored 0 when no ulcers were present, 1 when extent was <10%, 2 when extent is 10%-30%, and 3 when it is >30%. The variable extent of affected surface is scored 0 if none, 1 when <50%, 2 when 50%-75%, and 3 when >75%. The presence and type of narrowings is scored 0 when no narrowings are present, a single passable narrowing is scored 1, multiple passable narrowings are scored 2, and a nonpassable narrowing is scored 3. SES-CD between 0 and 2 suggested inactive disease, 3-6 is mildly active disease, 7-15 is moderately active disease, and >16 is severely active disease.

TABLE CXXXIX
Study 3 - 10 weeks SES-CD scores (LOCF)

| | Placebo | 200 mg | |
|---|---|---|---|
| | (N = 44) | (N = 128) | p-values |
| SES-CD mean, baseline | 15.9 | 14.2 | |
| SES-CD mean, W 10 | 13.2 | 11.6 | |
| SES-CD mean CFB, W 10 | −2.8 | −2.6 | 0.8725 |

TABLE CXL
Study 3 - 10 weeks SES-CD derived responses (LOCF)

| | Placebo | 200 mg | |
|---|---|---|---|
| | (N = 44) | (N = 128) | p-values |
| Endoscopic response | 18% | 25% | 0.4056 |
| (SES-CD % improvement ≥ 50) | | | |
| Endoscopic remission | 8% | 14% | 0.3721 |
| (SES-CD % improvement ≤ 4, | | | |
| ulcerated subscore ≤ 1 | | | |
| in all 5 segments) | | | |
| Mucosal healing | 2% | 2% | 0.9685 |
| (SES-CD % = 0) | | | |
| Deep remission | 5% | 8% | 0.6003 |
| (CDAI score < 150 points, | | | |
| SES-CD score ≤ 4, | | | |
| ulcerated subscore ≤ 1 | | | |
| in all 5 segments) | | | |

TABLE CXLI
Study 3 - 10 weeks SES-CD derived responses (LOCF) by prior TNF therapy

| | | | Anti-TNF experienced | |
|---|---|---|---|---|
| | Anti-TNF Naïve | | non-responders | |
| | Placebo | 200 mg | Placebo | 200 mg |
| | (N = 16) | (N = 57) | (N = 28) | (N = 71) |
| Endoscopic response | 25% | 28% | 14% | 23% |
| (SES-CD % improvement ≥ 50) | | | | |
| Endoscopic remission | 8% | 16% | 8% | 12% |
| (SES-CD % improvement ≤ 4, | | | | |
| ulcerated subscore ≤ 1 in all 5 segments) | | | | |
| Mucosal healing | 0% | 0% | 4% | 1% |
| (SES-CD % = 0) | | | | |
| Deep remission | 0% | 0% | 8% | 6% |
| (CDAI score < 150 points, | | | | |
| SES-CD score ≤ 4, | | | | |
| ulcerated subscore ≤ 1 in all 5 segments) | | | | |

TABLE CXLII
Study 3 - 10 weeks SES-CD derived responses (LOCF) by screening CRP Level

| | CRP ≤ 10 ml/L | | CRP ≥ 10 mg/L | |
|---|---|---|---|---|
| | Placebo | 200 mg | Placebo | 200 mg |
| | (N = 25) | (N = 74) | (N = 19) | (N = 54) |
| Endoscopic response | 20% | 23% | 16% | 28% |
| (SES-CD % improvement ≥ 50) | | | | |
| Endoscopic remission | 14% | 16% | 0% | 0% |
| (SES-CD % improvement ≤ 4, | | | | |
| ulcerated subscore ≤ 1 in all 5 segments) | | | | |
| Mucosal healing | 4% | 3% | 0% | 0% |
| (SES-CD % = 0) | | | | |
| Deep remission | 9% | 10% | 0% | 0% |
| (CDAI score < 150 points, | | | | |
| SES-CD score ≤ 4, | | | | |
| ulcerated subscore ≤ 1 in all 5 segments) | | | | |

TABLE CXLIII
Study 3 - 10 weeks SES-CD derived responses
(LOCF) by baseline corticosteroid use, LOCF

| | With steroids | | Without steroids | |
|---|---|---|---|---|
| | Placebo | 200 mg | Placebo | 200 mg |
| | (N = 23) | (N = 63) | (N = 21) | (N = 65) |
| Endoscopic response | 13% | 22% | 24% | 28% |
| (SES-CD % improvement ≥ 50) | | | | |
| Endoscopic remission | 5% | 12% | 11% | 15% |
| (SES-CD % improvement ≤ 4, | | | | |
| ulcerated subscore ≤ 1 in all 5 segments) | | | | |
| Mucosal healing | 0% | 0% | 5% | 5% |
| (SES-CD % = 0) | | | | |
| Deep remission | 0% | 0% | 11% | 5% |
| (CDAI score < 150 points, | | | | |
| SES-CD score ≤ 4, | | | | |
| ulcerated subscore ≤ 1 in all 5 segments) | | | | |

### 3.11.3. Histopathology Score

Histopathology data is collected from patients biopsy, and evaluated according to the procedure reported in D'haens et al., 1998.

TABLE CXLIV
Study 3 - Overall histopathology scores at week 10 (LOCF)

| | Placebo | 200 mg | |
|---|---|---|---|
| | (N = 44) | (N = 128) | p-values |
| Overall total score mean, | 26.5 | 22.8 | — |
| baseline | | | |
| Overall total score mean, | 25.5 | 19.1 | — |
| week 10 | | | |
| Overall total score mean, | −0.6 | −3.5 | 0.0359 |
| CFB at week 10 | | | |
| Overall total score mean, | 22% | −10% | — |
| % CFB at week 10 | | | |

TABLE CXLV
Study 3 - Overall histopathology scores at week 10 by prior TNF therapy (LOCF)

| | | | Anti-TNF experienced | |
|---|---|---|---|---|
| | Anti-TNF Naïve | | non-responders | |
| | Placebo | 200 mg | Placebo | 200 mg |
| | (N = 16) | (N = 57) | (N = 28) | (N = 71) |
| Overall total score mean, baseline | 22.7 | 20.3 | 28.8 | 25.0 |
| Overall total score mean, week 10 | 22.4 | 16.1 | 27.2 | 21.6 |
| Overall total score mean, CFB at week 10 | −0.3 | −3.9 | −0.7 | −3.2 |
| Overall total score mean, % CFB at week 10 | −10% | −14% | 39% | −7% |

TABLE CXLVI
Study 3 - Overall histopathology scores at week 10 by screening CRP Level (LOCF)

| | CRP ≤ 10 ml/L | | CRP ≥ 10 mg/L | |
|---|---|---|---|---|
| | Placebo | 200 mg | Placebo | 200 mg |
| | (N = 25) | (N = 74) | (N = 19) | (N = 54) |
| Overall total score mean, baseline | 21.1 | 21.8 | 32.7 | 24.3 |
| Overall total score mean, week 10 | 20.3 | 17.1 | 31.7 | 21.8 |
| Overall total score mean, CFB at week 10 | −0.1 | −4.8 | −1.1 | −1.8 |
| Overall total score mean, % CFB at week 10 | 42% | −20% | 2% | 4% |

TABLE CXLVII
Study 3 - Overall histopathology scores at week
10 by baseline corticosteroid use (LOCF)

| | With steroids | | Without steroids | |
|---|---|---|---|---|
| | Placebo | 200 mg | Placebo | 200 mg |
| | (N = 23) | (N = 63) | (N = 21) | (N = 65) |
| Overall total score mean, baseline | 33.3 | 24.9 | 18.7 | 20.7 |
| Overall total score mean, week 10 | 29.4 | 20.3 | 21.1 | 17.9 |
| Overall total score mean, CFB at week 10 | −3.9 | −4.3 | 3.3 | −2.7 |
| Overall total score mean, % CFB at week 10 | −11% | −15% | 65% | −5% |

### 3.11.4. Pharmacodynamics

CRP levels are determined as previously described in section 3.6

Fecal calprotectin is a protein belonging to the S100 family and occurring in large amounts in neutrophil granulocytes, where it accounts for 5% of total proteins and 60% of cytoplasm proteins. When inflammatory processes occur, calprotectin is released due to the degranulation of neutrophil granulocytes.

In bowel inflammation, calprotectin may be detected in the stool. The fecal levels provide direct information about the inflammation and is determined using a Calprest® enzyme immunoassay diagnostic kit (Eurospital Spa—Via Flavia 122-34147 Trieste—Italy; ref 9031).

TABLE CXLVIII
Study 3 - Mean CRP and fecal calprotectin
levels at week 10 (LOCF)

| | Placebo | 200 mg | |
|---|---|---|---|
| | (N = 44) | (N = 128) | p-values |
| Serum CRP mean, CFB mg/L | 4.9 | −1.0 | 0.0309 |
| Serum CRP mean, % CFB | 99% | 53% | — |
| Fecal calprotectin mean, | −58.7 | −86.7 | 0.4011 |
| CFB, mg/kg | | | |
| Fecal calprotectin mean, | 109% | 73% | — |
| % CFB | | | |

TABLE CXLIX
Study 3 - Median CRP and fecal calprotectin
levels at week 10 (LOCF)

| | Placebo | 200 mg |
|---|---|---|
| | (N = 44) | (N = 128) |
| Serum CRP median, CFB mg/L | 0.2 | −0.4 |
| Serum CRP median, % CFB | 1% | −10% |
| Fecal calprotectin median, | −15.5 | −75.5 |
| CFB, mg/kg | | |
| Fecal calprotectin median, | −1% | −29% |
| % CFB | | |

TABLE CL
Study 3 - normalized CRP and fecal calprotectin levels at week 10 (LOCF)

| | | Placebo | 200 mg | p- |
|---|---|---|---|---|
| | | (N = 44) | (N = 128) | values |
| Serum CRP normalizing | % subjects normalizing | 14% | 27% | 0.1938 |
| high to normal (<0.80 mg/dL) | Total subjects with high | 22 (50%) | 66 (52%) | — |
| | baseline (%) | | | |
| Fecal calprotectectin | % subjects normalizing | 5% | 12% | 0.1754 |
| normalizing high to normal | Total subjects with high | 42 (95%) | 124 (97%) | — |
| (<50 mg/kg) | baseline (%) | | | |
| Fecal calprotectectin | % subjects normalizing | 32% | 44% | 0.3002 |
| normalizing high to normal | Total subjects with high | 22 (50%) | 77 (60%) | — |
| (>250 mg/kg to ≤250 mg/kg) | baseline (%) | | | |

### Final Remarks

It will be appreciated by those skilled in the art that the foregoing descriptions are exemplary and explanatory in nature, and intended to illustrate the invention and its preferred embodiments. Through routine experimentation, an artisan will recognize apparent modifications and variations that may be made without departing from the spirit of the invention. All such modifications coming within the scope of the appended claims are intended to be included therein. Thus, the invention is intended to be defined not by the above description, but by the following claims and their equivalents.

All publications, including but not limited to patents and patent applications, cited in this specification are herein incorporated by reference as if each individual publication are specifically and individually indicated to be incorporated by reference herein as though fully set forth.

It should be understood that factors such as the differential cell penetration capacity of the various compounds can contribute to discrepancies between the activity of the compounds in the in vitro biochemical and cellular assays.

At least some of the chemical names of compound of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

### REFERENCES

Aletaha, D., Neogi, T., Silman, A. J., Funovits, J., Felson, D. T., Bingham, C. O., Birnbaum, N. S., Burmester, G. R., Bykerk, V. P., Cohen, M. D., et al. (2010). 2010 Rheumatoid arthritis classification criteria: an American College of Rheumatology/European League Against Rheumatism collaborative initiative. Arthritis Rheum. 62, 2569-2581.
Choy, E. H., and Panayi, G. S. (2001). Cytokine pathways and joint inflammation in rheumatoid arthritis. N. Engl. J. Med. 344, 907-916.
Daley-Yates, P. T., 2015. Inhaled corticosteroids: potency, dose equivalence and therapeutic index. Br. J. Clin. Pharmacol. 80, 372-380. doi:10.1111/bcp.12637
Felson, D. T., Anderson, J. J., Boers, M., Bombardier, C., Chernoff, M., Fried, B., Furst, D., Goldsmith, C., Kieszak, S., and Lightfoot, R. (1993). The American College of Rheumatology preliminary core set of disease activity measures for rheumatoid arthritis clinical trials. The Committee on Outcome Measures in Rheumatoid Arthritis Clinical Trials. Arthritis Rheum. 36, 729-740.
Firestein, G. S. (2003). Evolving concepts of rheumatoid arthritis. Nature 423, 356-361.
Freeman, H.-J. (2008). Use of the Crohn's disease activity index in clinical trials of biological agents. World J. Gastroenterol. WJG 14, 4127-4130.
Gasche, C., Lomer, M. C. E., Cavill, I., and Weiss, G. (2004). Iron, anaemia, and inflammatory bowel diseases. Gut 53, 1190-1197.
Kaltsonoudis, E., Papagoras, C., and Drosos, A. Current and Future Role of Methotrexate in the Therapeutic Armamentarium for Rheumatoid Arthritis. Int. J. Clin. Rheumatol. 7, 179-189.
Keystone, E. C., Taylor, P. C., Drescher, E., Schlichting, D. E., Beattie, S. D., Berclaz, P.-Y., Lee, C. H., Fidelus-Gort, R. K., Luchi, M. E., Rooney, T. P., et al. (2015). Safety and efficacy of baricitinib at 24 weeks in patients with rheumatoid arthritis who have had an inadequate response to methotrexate. Ann. Rheum. Dis. 74, 333-340.
Van t 'Klooster, G., Brys, R. C. X., Van, R., and Namour, F. S. (2013) Aminotriazolopyridine for Use in the Treatment of Inflammation, and Pharmaceutical Compositions Thereof
Kopf, M., Bachmann, M. F., and Marsland, B. J. (2010). Averting inflammation by targeting the cytokine environment. Nat. Rev. Drug Discov. 9, 703-718.
Lee, D. M., and Weinblatt, M. E. (2001). Rheumatoid arthritis. Lancet 358, 903-911.
Menet, C. J. M., and Smits, K. K. (2010). 5-Phenyl-[1,2,4]triazolo[1,5-A]pyridin-2-Y1Carboxamides as Jak Inhibitors.
O'Dell, J. R. (2004). Therapeutic Strategies for Rheumatoid Arthritis. N. Engl. J. Med. 350, 2591-2602.
O'Shea, J. J., Kontzias, A., Yamaoka, K., Tanaka, Y., and Laurence, A. (2013). Janus kinase inhibitors in autoimmune diseases. Ann. Rheum. Dis. 72 Suppl 2, ii111-ii115.
Pallis, A. G., Mouzas, I. A., Vlachonikolis, I. G., 2004. The inflammatory bowel disease questionnaire. A review of its national validation studies. Inflamm. Bowel Dis. 10,261-269. doi:10.1097/00054725-200405000-00014
Porter, R. (2011). The Merck Manual of Diagnosis and Therapy (Wiley).
Punwani, N., Scherle, P., Flores, R., Shi, J., Liang, J., Yeleswaram, S., Levy, R., Williams, W., and Gottlieb, A. (2012). Preliminary clinical activity of a topical JAK1/2 inhibitor in the treatment of psoriasis. J. Am. Acad. Dermatol. 67, 658-664.
Riese, R. J., Krishnaswami, S., and Kremer, J. (2010). Inhibition of JAK kinases in patients with rheumatoid arthritis: scientific rationale and clinical outcomes. Best Pract. Res. Clin. Rheumatol. 24, 513-526.
Smolen, J. S., and Steiner, G. (2003). Therapeutic strategies for rheumatoid arthritis. Nat. Rev. Drug Discov. 2, 473-488.
Smolen, J. S., Landewe, R., Breedveld, F. C., Buch, M., Burmester, G., Dougados, M., Emery, P., Gaujoux-Viala, C., Gossec, L., Nam, J., et al. (2014). EULAR recommendations for the management of rheumatoid arthritis with synthetic and biological disease-modifying antirheumatic drugs: 2013 update. Ann. Rheum. Dis. 73,492-509.
Tursi, A., Brandimarte, G., Papa, A., Giglio, A., Elisei, W., Giorgetti, G. M., Forti, G., Morini, S., Hassan, C., Pistoia, M. A., et al. (2010). Treatment of relapsing mild-to-moderate ulcerative colitis with the probiotic VSL#3 as adjunctive to a standard pharmaceutical treatment: a double-blind, randomized, placebo-controlled study. Am. J. Gastroenterol. 105, 2218-2227.
Vainchenker, W., Dusa, A., and Constantinescu, S. N. (2008). JAKs in pathology: role of Janus kinases in hematopoietic malignancies and immunodeficiencies. Semin. Cell Dev. Biol. 19, 385-393.
Wells, G., Becker, J.-C., Teng, J., Dougados, M., Schiff, M., Smolen, J., Aletaha, D., and van Riel, P. L. C. M. (2009). Validation of the 28-joint Disease Activity Score (DAS28) and European League Against Rheumatism response criteria based on C-reactive protein against disease progression in patients with rheumatoid arthritis, and comparison with the DAS28 based on erythrocyte sedimentation rate. Ann. Rheum. Dis. 68, 954-960.
Zenz, R., Eferl, R., Kenner, L., Florin, L., Hummerich, L., Mehic, D., Scheuch, H., Angel, P., Tschachler, E., and Wagner, E. F. (2005). Psoriasis-like skin disease and arthritis caused by inducible epidermal deletion of Jun proteins. Nature 437, 369-375.
Zikherman, J., and Weiss, A. (2011). Unraveling the functional implications of GWAS: how T cell protein tyrosine phosphatase drives autoimmune disease. J. Clin. Invest. 121, 4618-4621.
D'Haens, G. R., Geboes, K., Peeters, M., Baert, F., Penninckx, F., Rutgeerts, P., 1998. Early lesions of recurrent Crohn's disease caused by infusion of intestinal contents in excluded ileum. Gastroenterology 114,262-267. doi:10.1016/S0016-5085(98)70476-7
Sipponen, T., Nuutinen, H., Turunen, U., Farkkila, M., 2010. Endoscopic evaluation of Crohn's disease activity: comparison of the CDEIS and the SES-CD. Inflamm. Bowel Dis. 16,2131-2136. doi:10.1002/ibd.21300

1-22. (canceled)

23. A method for the treatment of inflammatory conditions, comprising:
administering to a patient in need thereof a compound according to Formula I: 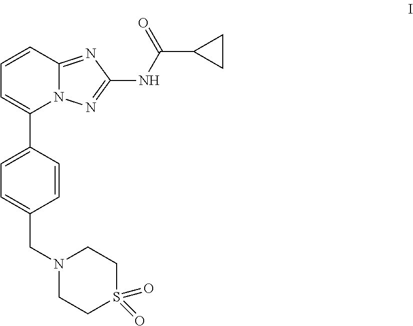 [SMILES: O=C(Nc1nc2cccc(-c3ccc(CN4CCS(=O)(=O)CC4)cc3)n2n1)C1CC1]
or a pharmaceutically acceptable salt thereof, or a solvate or the salt of a solvate thereof.

24. The method of claim 23, wherein the pharmaceutically acceptable salt of a solvate is the compound of Formula 1:HCl:3H₂O adduct.

25. The method of claim 23, wherein the compound is administered at a dose selected from 25 mg twice per day (b.i.d.), 50 mg once per day (q.d.), 50 mg b.i.d., 100 mg q.d., 100 mg b.i.d., and 200 mg q.d.

26. The method of claim 23, wherein the compound is administered once or twice per day for a period of at least 4 weeks.

27. The method of claim 23, wherein the compound is administered once or twice per day for a period of at least 8 weeks.

28. The method of claim 23, wherein the compound is administered once or twice per day for a period of at least 12 weeks.

29. The method of claim 23, wherein the compound is first administered at an induction dose selected from 100 mg twice per day (b.i.d.) and 200 mg once per day (q.d.) for a period of 4-12 weeks, followed by a maintenance dose selected from 50 mg b.i.d., 100 mg q.d., 100 mg b.i.d., and 200 mg q.d. for a period of at least 4 weeks.

30. The method of claim 29, wherein the induction dose is administered for 8-12 weeks.

31. The method of claim 29, wherein the induction dose is administered for 10 weeks.

32. The method of claim 29, wherein the induction dose is 200 mg q.d. for 10 weeks and the maintenance dose is 100 mg q.d. or 200 mg q.d. for a period of at least 4 weeks.

33. The method of claim 23, wherein the inflammatory condition is rheumatoid arthritis.

34. The method of claim 23, wherein the inflammatory condition is inflammatory bowel disease (IBD).

35. The method of claim 23, wherein the patient has previously had an insufficient response to methotrexate.

36. The method of claim 23, wherein the patient is concomitantly treated with methotrexate.

37. The method of claim 36, wherein the patient receives a dose of methotrexate ranging from 7.5 mg to 25 mg once per week.

38. The method of claim 37, wherein the dose of methotrexate ranges from 10 mg to 25 mg once per week.

39. The method of claim 23, wherein the patient is not concomitantly treated with methotrexate.

40. The method of claim 39, wherein the patient does not concomitantly receive any additional treatment for rheumatoid arthritis or inflammatory bowel disease (IBD).

41. The method of claim 23, wherein an increase in the patient's haemoglobin levels is seen after 4 weeks of treatment.

42. The method of claim 41, wherein an increase of at least 1 g/L is seen.

43. The method of claim 23, wherein the compound is administered at a dose of 100 mg twice per day (b.i.d.) or 200 mg once per day (q.d.) and wherein an increase in the patient's haemoglobin levels of at least 3.5 g/L is seen after four weeks of treatment.

44. A method for the treatment of inflammatory conditions, comprising:
administering to a patient in need thereof a compound according to Formula II: 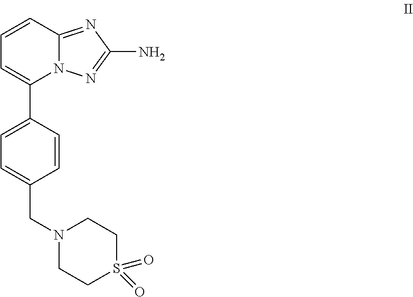 [SMILES: Nc1nc2cccc(-c3ccc(CN4CCS(=O)(=O)CC4)cc3)n2n1]
or a pharmaceutically acceptable salt thereof, or a solvate or the salt of a solvate thereof.

45. The method of claim 44, wherein the compound is administered at a dose selected from 25 mg twice per day (b.i.d.), 50 mg once per day (q.d.), 50 mg b.i.d., 100 mg q.d., 100 mg b.i.d., and 200 mg q.d.

46. The method of claim 44, wherein the compound is administered once or twice per day for a period of at least 4 weeks.

47. The method of claim 44, wherein the compound is administered once or twice per day for a period of at least 8 weeks.

48. The method of claim 44, wherein the compound is administered once or twice per day for a period of at least 12 weeks.

49. The method of claim 44, wherein the compound is first administered at an induction dose selected from 100 mg twice per day (b.i.d.) and 200 mg once per day (q.d.) for a period of 4-12 weeks, followed by a maintenance dose selected from 50 mg b.i.d., 100 mg q.d., 100 mg b.i.d., and 200 mg q.d. for a period of at least 4 weeks.

50. The method of claim 49, wherein the induction dose is administered for 8-12 weeks.

51. The method of claim 49, wherein the induction dose is administered for 10 weeks.

52. The method of claim 49, wherein the induction dose is 200 mg q.d. for 10 weeks and the maintenance dose is 100 mg q.d. or 200 mg q.d. for a period of at least 4 weeks.

53. The method of claim 44, wherein the inflammatory condition is rheumatoid arthritis.

54. The method of claim 44, wherein the inflammatory condition is inflammatory bowel disease (IBD).

55. The method of claim 44, wherein the patient has previously had an insufficient response to methotrexate.

56. The method of claim 44, wherein the patient is concomitantly treated with methotrexate.

57. The method of claim 56, wherein the patient receives a dose of methotrexate ranging from 7.5 mg to 25 mg once per week.

58. The method of claim 57, wherein the dose of methotrexate ranges from 10 mg to 25 mg once per week.

59. The method of claim 44, wherein the patient is not concomitantly treated with methotrexate.

60. The method of claim 59, wherein the patient does not concomitantly receive any additional treatment for rheumatoid arthritis or inflammatory bowel disease (IBD).

61. The method of claim 44, wherein an increase in the patient's haemoglobin levels is seen after 4 weeks of treatment.

62. The method of claim 61, wherein an increase of at least 1 g/L is seen.

63. The method of claim 44, wherein the compound is administered at a dose of 100 mg twice per day (b.i.d.) or 200 mg once per day (q.d.) and wherein an increase in the patient's haemoglobin levels of at least 3.5 g/L is seen after four weeks of treatment.
